(19) United States
(12) Patent Application Publication
(10) Pub. No.: US 2017/0327471 A1
(43) Pub. Date: Nov. 16, 2017

(54) Aryl sulphide and aryl sulphoxide derivatives having C-C-attached uracils as pesticides

(71) Applicant: BAYER CROPSCIENCE AKTIENGESELLSCHAFT, Monheim am Rhein (DE)

(72) Inventors: Bernd ALIG, Koenigswinter (DE); Silvia CEREZO-GALVEZ, Langenfeld (DE); Julia Johanna HAHN, Duesseldorf (DE); Kerstin ILG, Koeln (DE); Daniela PORTZ, Vettweiss (DE); Olga MALSAM, Roesrath (DE); Peter LOESEL, Leverkusen (DE); Ulrich GOERGENS, Ratingen (DE)

(21) Appl. No.: 15/509,640

(22) PCT Filed: Sep. 8, 2015
(86) PCT No.: PCT/EP2015/070423

§ 371 Date: Mar. 8, 2017

(30) Foreign Application Priority Data
Sep. 12, 2014 (EP) 14184619.6

(51) Int. Cl. C07D 239/56; A01N 43/54; C07D 239/54; C07D 239/553

(52) U.S. Cl. CPC C07D 239/56; C07D 239/553; A01N 43/54; C07D 239/54

(57) ABSTRACT

The invention relates to novel compounds of the formula (I)

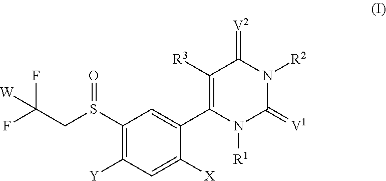

in which R¹, R², R³, n, V¹, V², W, X and Y have the meanings given in the description, a plurality of processes and intermediates for their preparation and their use as acaricides and/or insecticides and/or nematicides for controlling animal pests, for example in crop protection or in the field of animal health.

The present application relates to novel heterocyclic compounds, to processes for preparation thereof and to the use thereof for controlling animal pests, which include arthropods and especially insects.

Aryl sulphide and aryl sulphoxide derivatives and their insecticidal and acaricidal action have already been described in the literature, e. g. in WO 2013/157229, WO 2012/176856 and WO 1999/055668.

3-Aryl-6-arylpyrimidine-2,4(1H,3H)-diones are claimed as pesticides, for example, in JP 05043555, JP 05025144, JP 05025142 or EP 438209, as herbicides in JP 03215476.

EP 0166564 and JP 59181265 claim 1, 3-bis(alkyl)-6-[3-(methylsulphanyl)phenyl]pyrimidine-2,4(1H,3H)-diones, 1,3-bis(alkyl)-6-[3-(methylsulphinyl)phenyl]pyrimidine-2,4(1H,3H)-diones or their 4-thioxo derivatives as intermediates for preparing medicaments based on 4-aryliminouracils.

2-Thioxo-6-aryluracils are described as medicaments in WO 2013/068875.

Crop protection agents, which also include pesticides, have to meet many demands, for example in relation to efficacy, persistence, and spectrum of their action and possible use. Questions of toxicity and of combinability with other active compounds or formulation auxiliaries play a role, as does the question of the expense that the synthesis of an active compound requires. In addition, resistances can occur. For all these reasons, the search for novel crop protection agents cannot be considered to be complete, and there is a constant need for novel compounds having properties which, compared to the known compounds, are improved at least in relation to individual aspects.

It was an object of the present invention to provide compounds which widen the spectrum of the pesticides in various respects.

This object, and further objects which are not stated explicitly but can be discerned or derived from the connections discussed herein, are achieved by novel compounds of the formula (I)

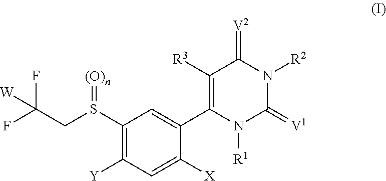

in which (Embodiment 1-1)
V¹ and V² independently of one another represent oxygen or represent sulphur;
R³ represents hydrogen, alkyl, halogen, haloalkyl, amino, cyano, carbamoyl, nitro, hydroxy, hydroxyalkyl, alkylthiosulphanyl, alkylsulphanyl, alkylsulphinyl, alkylsulphonyl, halogenalkylsulphanyl, halogenalkylsulphinyl, halogenalkylsulphonyl, cycloalkyl, alkylcarbonyl, haloalkylcarbonyl, alkoxycarbonyl, alkoxyalkyl, haloalkoxy or alkoxy;
R¹ and R² independently of one another represent hydrogen or alkyl; or
   represent alkenyl, alkynyl, cycloalkyl, cycloalkylalkyl, cycloalkenyl, cyanoalkyl, hydroxyalkyl, alkylcarbonyl, alkoxycarbonyl, alkoxycarbonylalkyl, haloalkylcarbonyl, alkoxyalkyl, haloalkoxyalkyl, alkoxy, haloalkoxy, haloalkyl, haloalkenyl or haloalkynyl, where the radicals mentioned above may optionally be substituted;
W represents hydrogen or halogen;
n represents the number 0, 1 or 2;
Y represents hydrogen, halogen, cyano, (C₁-C₆)-alkyl, (C₃-C₆)-cycloalkyl, (C₁-C₆)-haloalkyl, (C₁-C₆)-alkoxy or (C₁-C₆)-haloalkoxy;
X represents hydrogen, halogen, cyano, (C₁-C₆)-alkyl, (C₁-C₆)-haloalkyl or (C₁-C₆)-alkoxy.

A further embodiment (Embodiment 1-2) of the compounds of the formula (I) is characterized in that
V¹ and V² independently of one another represent oxygen or represent sulphur;
R³ represents hydrogen, alkyl, halogen, haloalkyl, amino, cyano, carbamoyl, nitro, hydroxy, hydroxyalkyl, alkylthiosulphanyl, alkylsulphanyl, alkylsulphinyl, alkylsulphonyl, haloalkylsulphanyl, haloalkylsulphinyl, haloalkylsulphonyl, cycloalkyl, alkylcarbonyl, haloalkylcarbonyl, alkoxycarbonyl, alkoxyalkyl, haloalkoxy or alkoxy;
R¹ and R² independently of one another represent hydrogen or alkyl; or
   represent alkenyl, alkynyl, cycloalkyl, cycloalkylalkyl, cycloalkenyl, cyanoalkyl, hydroxyalkyl, alkylcarbonyl, alkoxycarbonyl, alkoxycarbonylalkyl, haloalkylcarbonyl, alkoxyalkyl, haloalkoxyalkyl, alkoxy, haloalkoxy, haloalkyl, haloalkenyl or haloalkynyl, where the radicals mentioned above may optionally be substituted by fluorine, chlorine, bromine, iodine, alkyl, cycloalkyl, cyano, nitro, alkoxy, haloalkyl or haloalkoxy;
W represents hydrogen or halogen;
n represents the number 0, 1 or 2;
Y represents hydrogen, halogen, cyano, (C₁-C₆)-alkyl, (C₃-C₆)-cycloalkyl, (C₁-C₆)-haloalkyl, (C₁-C₆)-alkoxy or (C₁-C₆)-haloalkoxy;
X represents hydrogen, halogen, cyano, (C₁-C₆)-alkyl, (C₁-C₆)-haloalkyl or (C₁-C₆)-alkoxy.

It has been additionally found that the novel compounds of the formula (I) have good efficacy as pesticides, for example against arthropods and especially insects and acarids, and additionally generally have very good compatibility with plants, especially crop plants, and/or have favourable toxicological and/or environmentally relevant properties.

A general definition of the compounds of the invention is provided by the formula (I). Preferred substituents or ranges of the radicals listed under the formulae mentioned above and below are illustrated below (Embodiment 2-1):
V¹ and V² independently of one another represent oxygen or represent sulphur;
R³ represents hydrogen, halogen, amino, cyano, carbamoyl, nitro, hydroxy, (C₁-C₆)-alkyl, (C₁-C₆)-hydroxyalkyl, (C₁-C₆)-haloalkyl, (C₁-C₆)-alkylthiosulphanyl (C₁-C₆)-alkylsulphanyl, (C₁-C₆)-alkylsulphinyl, (C₁-C₆)-alkylsulphonyl, (C₁-C₆)-haloalkylsulphanyl, (C₁-C₆)-haloalkylsulphinyl, (C₁-C₆)-haloalkylsulphonyl, (C₃-C₆)-cycloalkyl, (C₁-C₆)-alkylcarbonyl, (C₁-C₆)-haloalkylcarbonyl, (C₁-C₆)-alkoxycarbonyl, (C₁-C₆)-alkoxyalkyl, (C₁-C₆)-haloalkoxy or (C₁-C₆)-alkoxy;
R¹ and R² independently of one another represent hydrogen or alkyl; or
   represent alkenyl, alkynyl, cycloalkyl, cycloalkylalkyl, cycloalkenyl, cyanoalkyl, hydroxyalkyl, alkylcarbonyl, haloalkylcarbonyl, alkoxyalkyl, haloalkoxyalkyl, alkoxy or haloalkoxy, where the radicals mentioned above may optionally be substituted; or
   represent haloalkyl, haloalkenyl or haloalkynyl;
W represents hydrogen or halogen;
n represents the number 0 or 1;
Y represents hydrogen, halogen, (C₁-C₆)-alkyl, (C₃-C₆)-cycloalkyl, (C₁-C₆)-haloalkyl, (C₁-C₆)-alkoxy or (C₁-C₆)-haloalkoxy;
X represents hydrogen, halogen, (C₁-C₆)-alkyl, (C₁-C₆)-haloalkyl or (C₁-C₆)-alkoxy.

Likewise preferred substituents or ranges of the radicals listed under the formulae mentioned above and below are illustrated below (Embodiment 2-2):
V¹ and V² independently of one another represent oxygen or represent sulphur;
R³ represents hydrogen, halogen, amino, cyano, carbamoyl, nitro, hydroxy, (C₁-C₆)-alkyl, (C₁-C₆)-hydroxyalkyl, (C₁-C₆)-haloalkyl, (C₁-C₆)-alkylthiosulphanyl (C₁-C₆)-alkylsulphanyl, (C₁-C₆)-alkylsulphinyl, (C₁-C₆)-alkylsulphonyl, (C₁-C₆)-haloalkylsulphanyl, (C₁-C₆)-haloalkylsulphinyl, (C₁-C₆)-haloalkylsulphonyl, (C₃-C₆)-cycloalkyl, (C₁-C₆)-alkylcarbonyl, (C₁-C₆)-haloalkylcarbonyl, (C₁-C₆)-alkoxycarbonyl, (C₁-C₆)-alkoxyalkyl, (C₁-C₆)-haloalkoxy or (C₁-C₆)-alkoxy;
R¹ and R² independently of one another represent hydrogen or alkyl; or
   represent alkenyl, alkynyl, cycloalkyl, cycloalkylalkyl, cycloalkenyl, cyanoalkyl, hydroxyalkyl, alkylcarbonyl, haloalkylcarbonyl, alkoxyalkyl, haloalkoxyalkyl, alkoxy or haloalkoxy, where the radicals mentioned above may optionally be substituted by fluorine, chlorine, bromine, iodine, (C₁-C₆)-alkyl, (C₃-C₆)-cycloalkyl, cyano, nitro, (C₁-C₆)-alkoxy, (C₁-C₆)-haloalkyl or (C₁-C₆)-haloalkoxy; or represent haloalkyl, haloalkenyl or haloalkynyl;
W represents hydrogen or halogen;
n represents the number 0 or 1;
Y represents hydrogen, halogen, (C₁-C₆)-alkyl, (C₃-C₆)-cycloalkyl, (C₁-C₆)-haloalkyl, (C₁-C₆)-alkoxy or (C₁-C₆)-haloalkoxy;
X represents hydrogen, halogen, (C₁-C₆)-alkyl, (C₁-C₆)-haloalkyl or (C₁-C₆)-alkoxy.

More preferred substituents or ranges for the radicals shown in the compounds of the formula (I) are illustrated below (Embodiment 3-1).
V¹ and V² independently of one another represent oxygen or represent sulphur;
R³ represents hydrogen, amino, halogen, cyano, (C₁-C₄)-alkyl, (C₁-C₄)-haloalkyl, (C₃-C₆)-cycloalkyl, (C₁-C₄)-alkylcarbonyl, (C₁-C₄)-haloalkylcarbonyl, (C₁-C₄)-alkoxycarbonyl or (C₁-C₄)-alkoxy;
R¹ and R² independently of one another represent hydrogen, (C₁-C₄)-alkyl, cyano-(C₁-C₄)-alkyl, (C₁-C₄)-haloalkyl, (C₁-C₄)-alkoxy-(C₁-C₄)-alkyl, (C₁-C₄)-haloalkoxy-(C₁-C₄)-alkyl, (C₁-C₄)-alkoxy or (C₁-C₄)-haloalkoxy; or
   represent (C₃-C₆)-cycloalkyl or (C₃-C₆)-cycloalkyl-(C₁-C₂)-alkyl, where the radicals mentioned above may optionally be substituted by fluorine, chlorine, bromine, iodine, (C₁-C₃)-alkyl, (C₃-C₆)-cycloalkyl, cyano, nitro, (C₁-C₄)-alkoxy, (C₁-C₃)-haloalkyl or (C₁-C₃)-haloalkoxy;
W represents hydrogen, chlorine or fluorine;
n represents the number 0 or 1;
Y represents halogen, (C₁-C₄)-alkyl, (C₃-C₆)-cycloalkyl, (C₁-C₄)-haloalkyl, (C₁-C₄)-alkoxy or (C₁-C₄)-haloalkoxy;
X represents hydrogen, halogen, (C₁-C₄)-alkyl, (C₁-C₄)-haloalkyl or (C₁-C₄)-alkoxy.

Particularly preferred substituents or ranges for the radicals shown in the compounds of the formula (I) are illustrated below (Embodiment 4-1).
V¹ and V² independently of one another represent oxygen or represent sulphur;
R³ represents hydrogen, chlorine, methyl, ethyl, methoxy, cyclopropyl, cyano or trifluoromethyl;
R¹ and R² independently of one another represent hydrogen, methyl, ethyl, n-propyl, isopropyl, n-butyl, sec-butyl, tert-butyl, CH₂CH(CH₃)₂, CH₂CN, CH₂CF₃, CH₂CHF₂, CH₂CH₂OCH₃, cyclopropyl, cyclobutyl or cyclopropylmethyl;
W represents fluorine;
n represents the number 0 or 1;
Y represents fluorine, chlorine, bromine, methyl, trifluoromethyl or methoxy;
X represents hydrogen, chlorine, fluorine or methyl.

Likewise particularly preferred substituents or ranges for the radicals shown in the compounds of the formula (I) are illustrated below (Embodiment 4-2).
V¹ and V² independently of one another represent oxygen or represent sulphur;
R³ represents hydrogen, chlorine, fluorine, methyl, ethyl, methoxy, cyclopropyl, cyano or trifluoromethyl;
R¹ and R² independently of one another represent hydrogen, methyl, ethyl, n-propyl, isopropyl, n-butyl, sec-butyl, tert-butyl, CH₂CH(CH₃)₂, CH₂CN, CH₂CF₃, CH₂CHF₂, CH₂CH₂OCH₃, cyclopropyl, cyclobutyl or cyclopropylmethyl;
W represents fluorine;
n represents the number 0 or 1;
Y represents fluorine, chlorine, bromine, methyl, trifluoromethyl or methoxy;
X represents hydrogen, chlorine, fluorine or methyl.

Likewise particularly preferred substituents or ranges for the radicals shown in the compounds of the formula (I) are illustrated below (Embodiment 4-3).
V¹ and V independently of one another represent oxygen or represent sulphur;
R³ represents hydrogen, fluorine or chlorine;
R¹ and R² independently of one another represent hydrogen, methyl, ethyl, n-propyl, isopropyl or CH₂CF₃;
W represents fluorine;
n represents the number 0 or 1;
Y represents methyl;
X represents fluorine or methyl.

Very particularly preferred substituents or ranges for the radicals shown in the compounds of the formula (I) are illustrated below (Embodiment 5-1).
V¹ and V² each represent oxygen;
R³ represents hydrogen;
R¹ and R² each represent methyl;
W represents fluorine;
n represents the number 0 or 1;
Y represents methyl;
X represents fluorine or methyl.

Very particularly preferred substituents or ranges for the radicals shown in the compounds of the formula (I) are illustrated below (Embodiment 5-2).
V¹ and V² each represent oxygen;
R³ represents hydrogen, fluorine or chlorine;
R¹ and R² each represent methyl;
W represents fluorine;
n represents the number 0 or 1;
Y represents methyl;
X represents fluorine or methyl.

In the broadest and the preferred definitions, unless stated otherwise, halogen is selected from the group of fluorine, chlorine, bromine and iodine, preferably in turn from the group of fluorine, chlorine and bromine.

In the more preferred and optionally the particularly preferred and very particularly preferred definitions, unless stated otherwise, halogen is selected from the group of fluorine, chlorine, bromine and iodine, preferably in turn from the group of fluorine, chlorine and bromine.

Halogen-substituted radicals, for example haloalkyl, are mono- or polyhalogenated up to the maximum possible number of substituents. In the case of polyhalogenation, the halogen atoms may be the same or different. Halogen represents fluorine, chlorine, bromine and iodine, especially fluorine, chlorine and bromine.

Saturated or unsaturated hydrocarbon radicals, such as alkyl or alkenyl, may each be straight-chain or branched if possible, including in combination with heteroatoms, as, for example, in alkoxy.

Optionally substituted radicals may be mono- or polysubstituted, where the substituents in the case of polysubstitution may be the same or different. If substituents are intended or optionally intended, the substituents are, unless indicated otherwise, halogen, alkyl, cycloalkyl, cyano, nitro, alkoxy, haloalkyl or haloalkoxy, in particular fluorine, chlorine, (C₁-C₃)-alkyl, (C₃-C₆)-cycloalkyl (especially cyclopropyl), cyano, (C₁-C₄)-alkoxy, (C₁-C₃)-haloalkyl or (C₁-C₃)-haloalkoxy.

The radical definitions or elucidations given above in general terms or within ranges of preference apply correspondingly to the end products (including the compounds of the formula (I) with the substructures (I-A), (I-A-1), (I-B), (I-B-1), (I-C), (I-C-1), (I-D) and (I-D-1), which will be elucidated later), and to the starting materials and intermediates. These radical definitions can be combined with one another as desired, i.e. including combinations between the respective ranges of preference.

Preference is given in accordance with the invention to compounds of the formula (I) in which a combination of the definitions given above as preferred is present, and every embodiment described above as preferred constitutes an independent combination, in particular a combination as described in Embodiment 2-1 or in Embodiment 2-2.

More preference is given in accordance with the invention to compounds of the formula (I) in which a combination of the definitions given above as more preferred is present, and every embodiment described above as more preferred constitutes an independent combination, in particular a combination as described in Embodiment 3-1.

Particular is given in accordance with the invention to compounds of the formula (I) in which a combination of the definitions given above as particularly preferred is present, and every embodiment described above as particularly preferred constitutes an independent combination, in particular a combination as described in Embodiment 4-1 or in Embodiment 4-2 or in Embodiment 4-3.

Very particular preference is given in accordance with the invention to compounds of the formula (I) in which a combination of the definitions given above as very particularly preferred is present, and every embodiment described above as very particularly preferred constitutes an independent combination, in particular a combination as described in Embodiment 5-1 or in Embodiment 5-2.

Furthermore, X and Y represent in particular the following combinations (Y,X): (Me, F), (Me,H), (Me,Cl), (Me,Me), (Cl,Cl), (Cl,F), (MeO,F), (MeO,H), (Cl,H), (Br,H), (Br,F), (F,F), (CF₃,H), (CF₃,F), with particular preference being given to the following combinations (Y,X): (Me, F), (Me,Me), (Cl,Cl).

In further preferred embodiments, the invention relates to compounds of the formula (I) in which V¹ and V² represent oxygen. This results in compounds of the formula (I-A)

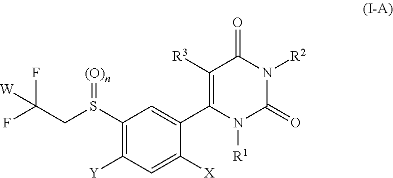

In the compounds of the formula (I) defined by the structure (I-A), the radicals or structural elements R¹, R², R³, n, W, X and Y have the meanings described above, in particular as described in Embodiment 1-1 (Embodiment I-A.1-1) or in Embodiment 1-2 (Embodiment I-A.1-2).

Preferred from among the compounds of the formula (I) defined by structure (I-A) are those compounds in which a combination of the meanings given above as preferred is present, and every embodiment described above as preferred constitutes an independent combination, in particular a combination as described in Embodiment 2-1 (Embodiment I-A.2-1) or in Embodiment 2-2 (Embodiment I-A.2-2).

More preferred from among the compounds of the formula (I) defined by structure (I-A) are those compounds in which a combination of the meanings given above as more preferred is present, and every embodiment described above as more preferred constitutes an independent combination, in particular a combination as described in Embodiment 3-1 (Embodiment I-A.3-1).

Particularly preferred from among the compounds of the formula (I) defined by structure (I-A) are those compounds in which a combination of the meanings given above as particularly preferred is present, and every embodiment described above as particularly preferred constitutes an independent combination, in particular a combination as described in Embodiment 4-1 (Embodiment I-A.4-1) or in Embodiment 4-2 (Embodiment I-A.4-2) or in Embodiment 4-3 (Embodiment I-A.4-3).

Very particularly preferred from among the compounds of the formula (I) defined by structure (I-A) are those compounds in which a combination of the meanings given above as very particularly preferred is present, and every embodiment described above as very particularly preferred constitutes an independent combination, in particular a combination as described in Embodiment 5-1 (Embodiment I-A.5-1) or in Embodiment 5-2 (Embodiment I-A.5-2).

More particularly, X and Y represent the following (Y,X) combinations: (Me, F), (Me,H), (Me,Cl), (Me,Me), (Cl,Cl), (Cl,F), (MeO,F), (MeO,H), (Cl,H), (Br,H), (Br,F), (F,F), (CF₃,H), (CF₃,F), particular preference being given to the following (Y,X) combinations: (Me, F), (Me,Me), (Cl,Cl).

Particularly preferred embodiments of compounds of the formula (I-A) are those in which R³ represents H. This results in compounds of the formula (I-A-1).

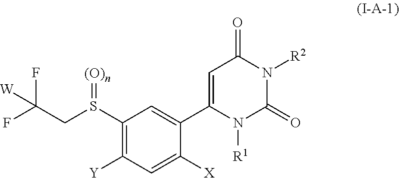

In the compounds of the formula (I) defined by the structure (I-A-1), the radicals or structural elements R¹, R², n, W, X and Y have the meanings described above, in particular as described in Embodiment 1-1 (Embodiment I-A-1.1-1) or in Embodiment 1-2 (Embodiment I-A-1.1-2).

Preferred from among the compounds of the formula (I) defined by structure (I-A-1) are those compounds in which a combination of the meanings given above as preferred is present, and every embodiment described above as preferred constitutes an independent combination, in particular a combination as described in Embodiment 2-1 (Embodiment I-A-1.2-1) or in Embodiment 2-2 (Embodiment I-A-1.2-2).

More preferred from among the compounds of the formula (I) defined by structure (I-A-1) are those compounds in which a combination of the meanings given above as more preferred is present, and every embodiment described above as more preferred constitutes an independent combination, in particular a combination as described in Embodiment 3-1 (Embodiment I-A-1.3-1).

Particularly preferred from among the compounds of the formula (I) defined by structure (I-A-1) are those compounds in which a combination of the meanings given above as particularly preferred is present, and every embodiment described above as particularly preferred constitutes an independent combination, in particular a combination as described in Embodiment 4-1 (Embodiment I-A-1.4-1) or in Embodiment 4-2 (Embodiment I-A-1.4-2) or in Embodiment 4-3 (Embodiment I-A-1.4-3).

Very particularly preferred from among the compounds of the formula (I) defined by structure (I-A-1) are those compounds in which a combination of the meanings given above as very particularly preferred is present, and every embodiment described above as very particularly preferred constitutes an independent combination, in particular a combination as described in Embodiment 5-1 (Embodiment I-A-1.5-1) or in Embodiment 5-2 (Embodiment I-A-1.5-2).

More particularly, X and Y represent the following (Y,X) combinations: (Me, F), (Me,H), (Me,Cl), (Me,Me), (Cl,Cl), (Cl,F), (MeO,F), (MeO,H), (Cl,H), (Br,H), (Br,F), (F,F), (CF₃,H), (CF₃,F), particular preference being given to the following (Y,X) combinations: (Me, F), (Me,Me), (Cl,Cl).

In further preferred embodiments, the invention relates to compounds of the formula (I) in which V¹ represents sulphur and V² represents oxygen. This results in compounds of the formula (I-B).

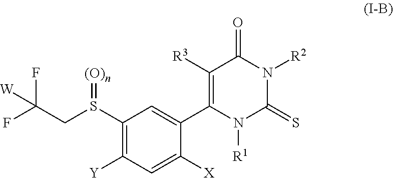

In the compounds of the formula (I) defined by the structure (I-B), the radicals or structural elements R¹, R², R³, n, W, X and Y have the meanings described above, in particular as described in Embodiment 1-1 (Embodiment I-B.1-1) or in Embodiment 1-2 (Embodiment I-B.1-2).

Preferred from among the compounds of the formula (I) defined by structure (I-B) are those compounds in which a combination of the meanings given above as preferred is present, and every embodiment described above as preferred constitutes an independent combination, in particular a combination as described in Embodiment 2-1 (Embodiment I-B.2-1) or in Embodiment 2-2 (Embodiment I-B.2-2).

More preferred from among the compounds of the formula (I) defined by structure (I-B) are those compounds in which a combination of the meanings given above as more preferred is present, and every embodiment described above as more preferred constitutes an independent combination, in particular a combination as described in Embodiment 3-1 (Embodiment I-B.3-1).

Particularly preferred from among the compounds of the formula (I) defined by structure (I-B) are those compounds in which a combination of the meanings given above as particularly preferred is present, and every embodiment described above as particularly preferred constitutes an independent combination, in particular a combination as described in Embodiment 4-1 (Embodiment I-B.4-1) or in Embodiment 4-2 (Embodiment I-B.4-2) or in Embodiment 4-3 (Embodiment I-B.4-3).

Very particularly preferred from among the compounds of the formula (I) defined by structure (I-B) are those compounds in which a combination of the meanings given above as very particularly preferred is present, and every embodiment described above as very particularly preferred constitutes an independent combination, in particular a combination as described in Embodiment 5-1 (Embodiment I-B.5-1) or in Embodiment 5-2 (Embodiment I-B.5-2).

More particularly, X and Y represent the following (Y,X) combinations: (Me, F), (Me,H), (Me,Cl), (Me,Me), (Cl,Cl), (Cl,F), (MeO,F), (MeO,H), (Cl,H), (Br,H), (Br,F), (F,F), (CF₃,H), (CF₃,F), particular preference being given to the following (Y,X) combinations: (Me, F), (Me,Me), (Cl,Cl).

Particularly preferred embodiments of compounds of the formula (I-B) are those in which R³ represents H. This results in compounds of the formula (I-B-1).

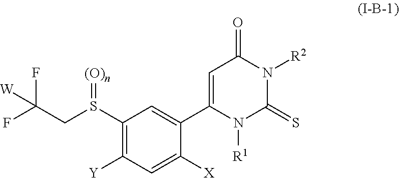

In the compounds of the formula (I) defined by the structure (I-B-1), the radicals or structural elements R¹, R², n, W, X and Y have the meanings described above, in particular as described in Embodiment 1-1 (Embodiment I-B-1.1-1) or in Embodiment 1-2 (Embodiment I-B-1.1-2).

Preferred from among the compounds of the formula (I) defined by structure (I-B-1) are those compounds in which a combination of the meanings given above as preferred is present, and every embodiment described above as preferred constitutes an independent combination, in particular a combination as described in Embodiment 2-1 (Embodiment I-B-1.2-1) or in Embodiment 2-2 (Embodiment I-B-1.2-2).

More preferred from among the compounds of the formula (I) defined by structure (I-B-1) are those compounds in which a combination of the meanings given above as more preferred is present, and every embodiment described above as more preferred constitutes an independent combination, in particular a combination as described in Embodiment 3-1 (Embodiment I-B.1.3-1).

Particularly preferred from among the compounds of the formula (I) defined by structure (I-B-1) are those compounds in which a combination of the meanings given above as particularly preferred is present, and every embodiment described above as particularly preferred constitutes an independent combination, in particular a combination as described in Embodiment 4-1 (Embodiment I-B-1.4-1) or in Embodiment 4-2 (Embodiment I-B-1.4-2) or in Embodiment 4-3 (Embodiment I-B-1.4-3).

Very particularly preferred from among the compounds of the formula (I) defined by structure (I-B-1) are those compounds in which a combination of the meanings given above as very particularly preferred is present, and every embodiment described above as very particularly preferred constitutes an independent combination, in particular a combination as described in Embodiment 5-1 (Embodiment I-B-1.5-1) or in Embodiment 5-2 (Embodiment I-B-1.5-2).

More particularly, X and Y represent the following (Y,X) combinations: (Me, F), (Me,H), (Me,Cl), (Me,Me), (Cl,Cl), (Cl,F), (MeO,F), (MeO,H), (Cl,H), (Br,H), (Br,F), (F,F), (CF₃,H), (CF₃,F), particular preference being given to the following (Y,X) combinations: (Me, F), (Me,Me), (Cl,Cl).

In further preferred embodiments, the invention relates to compounds of the formula (I) in which V¹ represents oxygen and V² represents sulphur. This results in compounds of the formula (I-C).

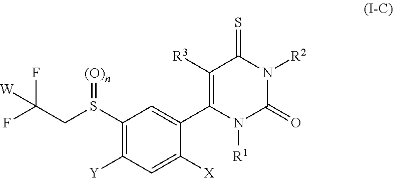

In the compounds of the formula (I) defined by the structure (I-C), the radicals or structural elements R¹, R², R³, n, W, X and Y have the meanings described above, in particular as described in Embodiment 1-1 (Embodiment I-C.1-1) or in Embodiment 1-2 (Embodiment I-C.1-2).

Preferred from among the compounds of the formula (I) defined by structure (I-C) are those compounds in which a combination of the meanings given above as preferred is present, and every embodiment described above as preferred constitutes an independent combination, in particular a combination as described in Embodiment 2-1 (Embodiment I-C.2-1) or in Embodiment 2-2 (Embodiment I-C.2-2).

More preferred from among the compounds of the formula (I) defined by structure (I-C) are those compounds in which a combination of the meanings given above as more preferred is present, and every embodiment described above as more preferred constitutes an independent combination, in particular a combination as described in Embodiment 3-1 (Embodiment I-C.3-1).

Particularly preferred from among the compounds of the formula (I) defined by structure (I-C) are those compounds in which a combination of the meanings given above as particularly preferred is present, and every embodiment described above as particularly preferred constitutes an independent combination, in particular a combination as described in Embodiment 4-1 (Embodiment I-C.4-1) or in Embodiment 4-2 (Embodiment I-C.4-2) or in Embodiment 4-3 (Embodiment I-C.4-3).

Very particularly preferred from among the compounds of the formula (I) defined by structure (I-C) are those compounds in which a combination of the meanings given above as very particularly preferred is present, and every embodiment described above as very particularly preferred constitutes an independent combination, in particular a combination as described in Embodiment 5-1 (Embodiment I-C.5-1) or in Embodiment 5-2 (Embodiment I-C.5-2).

More particularly, X and Y represent the following (Y,X) combinations: (Me, F), (Me,H), (Me,Cl), (Me,Me), (Cl,Cl), (Cl,F), (MeO,F), (MeO,H), (Cl,H), (Br,H), (Br,F), (F,F), (CF₃,H), (CF₃,F), particular preference being given to the following (Y,X) combinations: (Me, F), (Me,Me), (Cl,Cl).

Particularly preferred embodiments of compounds of the formula (I-C) are those in which R³ represents H. This results in compounds of the formula (I-C-1).

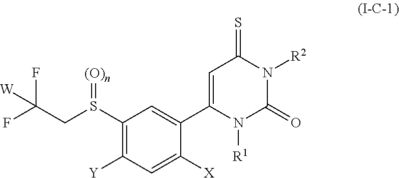

In the compounds of the formula (I) defined by the structure (I-C-1), the radicals or structural elements R¹, R², n, W, X and Y have the meanings described above, in particular as described in Embodiment 1-1 (Embodiment I-C-1.1-1) or in Embodiment 1-2 (Embodiment I-C-1.1-2).

Preferred among the compounds of the formula (I) defined by structure (I-C-1) are those compounds in which a combination of the meanings given above as preferred is present, and every embodiment described above as preferred constitutes an independent combination, in particular a combination as described in Embodiment 2-1 (Embodiment I-C-1.2-1) or in Embodiment 2-2 (Embodiment I-C-1.2-2).

More preferred from among the compounds of the formula (I) defined by structure (I-C-1) are those compounds in which a combination of the meanings given above as more preferred is present, and every embodiment described above as more preferred constitutes an independent combination, in particular a combination as described in Embodiment 3-1 (Embodiment I-C-1.3-1).

Particularly preferred from among the compounds of the formula (I) defined by structure (I-C-1) are those compounds in which a combination of the meanings given above as particularly preferred is present, and every embodiment described above as particularly preferred constitutes an independent combination, in particular a combination as described in Embodiment 4-1 (Embodiment I-C-1.4-1) or in Embodiment 4-2 (Embodiment I-C-1.4-2) or in Embodiment 4-3 (Embodiment I-C-1.4-3).

Very particularly preferred from among the compounds of the formula (I) defined by structure (I-C-1) are those compounds in which a combination of the meanings given above as very particularly preferred is present, and every embodiment described above as very particularly preferred constitutes an independent combination, in particular a combination as described in Embodiment 5-1 (Embodiment I-C-1.5-1) or in Embodiment 5-2 (Embodiment I-C-1.5-2).

More particularly, X and Y represent the following (Y,X) combinations: (Me, F), (Me,H), (Me,Cl), (Me,Me), (Cl,Cl), (Cl,F), (MeO,F), (MeO,H), (Cl,H), (Br,H), (Br,F), (F,F), (CF₃,H), (CF₃,F), particular preference being given to the following (Y,X) combinations: (Me, F), (Me,Me), (Cl,Cl).

In further preferred embodiments, the invention relates to compounds of the formula (I) in which V¹ and V² represent sulphur. This results in compounds of the formula (I-D).

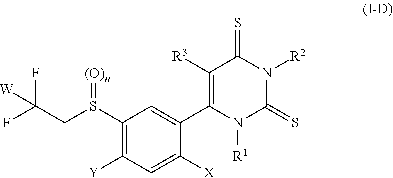

In the compounds of the formula (I) defined by the structure (I-D), the radicals or structural elements R¹, R², R³, n, W, X and Y have the meanings described above, in particular as described in Embodiment 1-1 (Embodiment I-D. 1-1) or in Embodiment 1-2 (Embodiment I-D.1-2).

Preferred from among the compounds of the formula (I) defined by structure (I-D) are those compounds in which a combination of the meanings given above as preferred is present, and every embodiment described above as preferred constitutes an independent combination, in particular a combination as described in Embodiment 2-1 (Embodiment I-D.2-1) or in Embodiment 2-2 (Embodiment I-D.2-2).

More preferred from among the compounds of the formula (I) defined by structure (I-D) are those compounds in which a combination of the meanings given above as more preferred is present, and every embodiment described above as more preferred constitutes an independent combination, in particular a combination as described in Embodiment 3-1 (Embodiment I-D.3-1).

Particularly preferred from among the compounds of the formula (I) defined by structure (I-D) are those compounds in which a combination of the meanings given above as particularly preferred is present, and every embodiment described above as particularly preferred constitutes an independent combination, in particular a combination as described in Embodiment 4-1 (Embodiment I-D.4-1) or in Embodiment 4-2 (Embodiment I-D.4-2) or in Embodiment 4-3 (Embodiment I-D.4-3).

Very particularly preferred from among the compounds of the formula (I) defined by structure (I-D) are those compounds in which a combination of the meanings given above as very particularly preferred is present, and every embodiment described above as very particularly preferred constitutes an independent combination, in particular a combination as described in Embodiment 5-1 (Embodiment I-D.5-1) or in Embodiment 5-2 (Embodiment I-D.5-2).

More particularly, X and Y represent the following (Y,X) combinations: (Me, F), (Me,H), (Me,Cl), (Me,Me), (Cl,Cl), (Cl,F), (MeO,F), (MeO,H), (Cl,H), (Br,H), (Br,F), (F,F), (CF₃,H), (CF₃,F), particular preference being given to the following (Y,X) combinations: (Me, F), (Me,Me), (Cl,Cl).

Particularly preferred embodiments of the compounds of the formula (I-D) are those in which R³ represents H. This results in compounds of the formula (I-D-1).

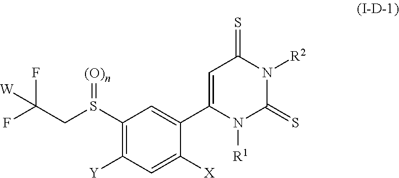

In the compounds of the formula (I) defined by the structure (I-D-1), the radicals or structural elements R¹, R², n, W, X and Y have the meanings described above, in particular as described in Embodiment 1-1 (Embodiment I-D-1.1-1) or in Embodiment 1-2 (Embodiment I-D-1.1-2).

Preferred from among the compounds of the formula (I) defined by structure (I-D-1) are those compounds in which a combination of the meanings given above as preferred is present, and every embodiment described above as preferred constitutes an independent combination, in particular a combination as described in Embodiment 2-1 (Embodiment I-D-1.2-1) or in Embodiment 2-2 (Embodiment I-D-1.2-2).

More preferred from among the compounds of the formula (I) defined by structure (I-D-1) are those compounds in which a combination of the meanings given above as more preferred is present, and every embodiment described above as more preferred constitutes an independent combination, in particular a combination as described in Embodiment 3-1 (Embodiment I-D-1.3-1).

Particularly preferred from among the compounds of the formula (I) defined by structure (I-D-1) are those compounds in which a combination of the meanings given above as particularly preferred is present, and every embodiment described above as particularly preferred constitutes an independent combination, in particular a combination as described in Embodiment 4-1 (Embodiment I-D-1.4-1) or in Embodiment 4-2 (Embodiment I-D-1.4-2) or in Embodiment 4-3 (Embodiment I-D-1.4-3).

Very particularly preferred from among the compounds of the formula (I) defined by structure (I-D-1) are those compounds in which a combination of the meanings given above as very particularly preferred is present, and every embodiment described above as very particularly preferred constitutes an independent combination, in particular a combination as described in Embodiment 5-1 (Embodiment I-D-1.5-1) or in Embodiment 5-2 (Embodiment I-D-1.5-2).

More particularly, X and Y represent the following (Y,X) combinations: (Me, F), (Me,H), (Me,Cl), (Me,Me), (Cl,Cl), (Cl,F), (MeO,F), (MeO,H), (Cl,H), (Br,H), (Br,F), (F,F), (CF₃,H), (CF₃,F), particular preference being given to the following (Y,X) combinations: (Me, F), (Me,Me), (Cl,Cl).

The compounds of the formula (I) can also be present as salts, in particular acid addition salts and metal salt complexes. The compounds of the formula (I) and the acid addition salts and metal salt complexes thereof have good efficacy, especially for controlling animal pests, which include arthropods and in particular insects and acarids.

Suitable salts of the compounds of the general formula (I) include customary nontoxic salts, i.e. salts with appropriate bases and salts with added acids. Preference is given to salts with inorganic bases, such as alkali metal salts, for example sodium, potassium or caesium salts, alkaline earth metal salts, for example calcium or magnesium salts, ammonium salts, salts with organic bases and with inorganic amines, for example triethylammonium, dicyclohexylammonium, N,N′-dibenzylethylenediammonium, pyridinium, picolinium or ethanolammonium salts, salts with inorganic acids, for example hydrochlorides, hydrobromides, dihydrosulphates, trihydrosulphates, or phosphates, salts with organic carboxylic acids or organic sulphonic acids, for example formates, acetates, trifluoroacetates, maleates, tartrates, methanesulphonates, benzenesulphonates or para-toluenesulphonates, salts with basic amino acids, for example arginates, aspartates or glutamates, and the like.

The compounds of the formula (I) may possibly also, depending on the nature of the substituents, be in the form of stereoisomers, i.e. in the form of geometric and/or optical isomers or isomer mixtures of varying composition. This invention provides both the pure stereoisomers and any desired mixtures of these isomers, even though it is generally only compounds of the formula (I) that are discussed here.

However, preference is given in accordance with the invention to using the optically active, stereoisomeric forms of the compounds of the formula (I) and salts thereof.

Enantiomers may result in particular with respect to the chiral sulphur atom in compounds of the formula (I) according to the invention.

The invention therefore relates both to the pure enantiomers and diastereomers and to mixtures thereof for controlling animal pests, including arthropods and especially insects and acarids. An individual configuration of the invention is therefore directed to the presence of the R enantiomer, in particular with respect to the chiral sulphur atom, or to a mixture comprising a majority of the R enantiomer, preferably where the ratio of R to S enantiomer is at least 60:40 and, with increasing preference, at least 70:30, 75:25, 80:20, 85:15 and 90:10. A further individual configuration of the invention is therefore directed to the presence of the S enantiomer, in particular with respect to the chiral sulphur atom, or to a mixture comprising a majority of the S enantiomer, preferably where the ratio of S to R enantiomer is at least 60:40 and, with increasing preference, at least 70:30, 75:25, 80:20, 85:15 and 90:10.

The compounds of the formula (I) may be present in different polymorphic forms or as a mixture of different polymorphic forms. Both the pure polymorphs and the polymorph mixtures form part of the subject-matter of the invention and can be used in accordance with the invention.

The compounds according to the invention are defined in general terms by the formula (I), which includes all the possible rotamers and mixtures thereof.

The compounds of the formula (I) according to the invention can be prepared by customary methods known to those skilled in the art. Various preparation processes, which also form part of the subject-matter of the invention, are described below.

### Preparation Processes

The compounds of the formula (I) according to the invention can be obtained by the processes shown in the following schemes:

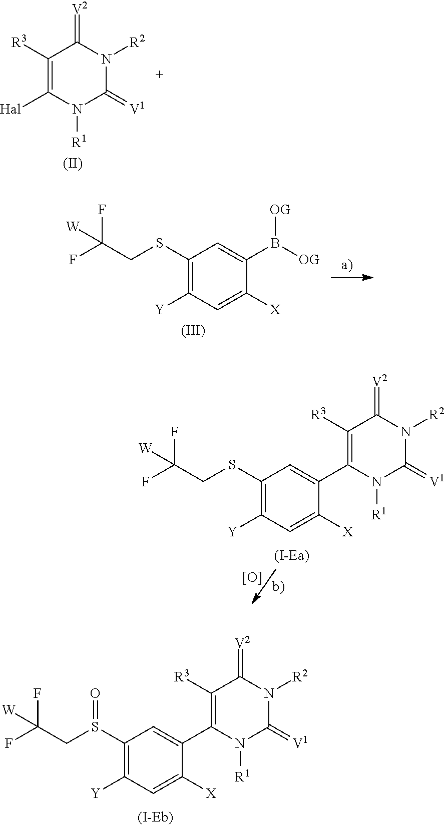

The radicals R¹, R², R³, V¹, V², W, X and Y have the meanings described above.

Hal represents chlorine, bromine or iodine.

G represents hydrogen, (C₁-C₆)-alkyl, (C₂-C₆)-alkenyl (preferably hydrogen, (C₁-C₄)-alkyl, (C₂-C₄)-alkenyl; particularly preferably hydrogen)
or B(OG)₂ together forms a 5- or 6-membered cycle, preferably a 5-membered cycle

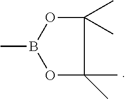

### Step (a)

In one embodiment of the invention, 6-halo(thio)uracils of the formula (II) are reacted with boric acids or 1,3,2-dioxaborolanes of the formula (III) to give the compounds of the formula (I-Ea) according to the invention.

This reaction type—Pd-catalysed reactions of boric acids or 1,3,2-dioxaborolanes with halogenated uracils—is known from the literature, for example from Tetrahedron, 2011, 67, 3915-3923 or Tetrahedron, 2002, 58, 10137-10143.

In WO 2013/068875, 6-iodo-1-R-2-(methylsulphanyl)pyrimidin-4(1H)-ones are reacted with arylboronic acids to give 1-R-2-(methylsulphanyl)-6-arylpyrimidin-4(1H)-ones. After reaction with ammonium sulphide in pyridine, this affords 1-R-6-aryl-2-thioxo-2,3-dihydropyrimidin-4(1H)-ones. This reaction type and similar variants are also described in WO 2013/068875.

Direct regioselective Pd-catalysed arylations of uracils with arylboronic acids are described, for example, in Tetrahedron Lett., 2014, 55, 1077-1081 or J. Org. Chem., 2011, 5309-5319.

Corresponding reactions of 6-iodouracils with arylstannanes to give 6-aryluracils are described in the literature, for example in Tetrahedron, 1993, 49, 2533-2542.

Many 6-halo(thio)uracils of the formula (II) are commercially available, for example 6-chloro-1,3-dimethyluracil (Aldrich), or they can be prepared by known processes.

WO 2013/068875 describes the synthesis of 6-iodo(thio)uracils by reacting (thio)uracils and iodine in the presence of lithium diisopropylamide; analogously, in J Organomet. Chem., 696, 2011, 1089-1095 1-alkyluracils are converted into 6-iodo-1-alkyluracils.

5-Fluoro-6-halouracils are claimed as intermediates in JP 60190768 and JP 61145167, or they can be prepared analogously to J. Org. Chem., 1986, 5149 or Chemistry Lett., 1984, 9, 1573-1576.

US 2008/013410 or DE 10301788 describe the syntheses of 6-chloro-3-alkylpyrimidine-2,4(1H,3H)diones. Their subsequent alkylation, for example using iodomethane in dimethylformamide and K₂CO₃ as base, affords 6-chloro-1-methyl-3-alkylpyrimidine-2,4(1H,3H)-diones.

Alternatively, WO 2003/002567 describes the synthesis of 6-chloro-1-alkylpyrimidine-2,4(1H,3H)-diones. Their subsequent alkylation, for example using iodomethane in dimethylformamide and K₂CO₃ as base, affords 6-chloro-1-alkyl-3-methylpyrimidine-2,4(1H,3H)-dione.

Further alkylations of 6-halouracils with alkyl halides are described in Europ. J. Med. Chem., 68, 2013, 312-320 or JP 62022717, the use of dimethyl sulphate or diethyl sulphate as agent for alkylating 6-halouracils is described in Org. Biomol. Chem., 2012, 10, 8860.

The boric acids or 1,3,2-dioxaborolanes of the formula (III) required as starting materials are commercially available, for example {2-fluoro-4-methyl-5-[(2,2,2-trifluoroethyl)sulphanyl]phenyl}boric acid (Aces Pharma, USA), [CAS-Reg. No. 248270-00-0], or are known from other patent specifications, for example from WO 2013/157229, or they can be synthesized by processes known from the literature.

The catalysts used in the reactions of the 6-halo(thio)uracils (II) preferably consist of salts or complexes of palladium, copper or nickel.

Suitable palladium catalysts are, for example, palladium(II) acetate, tetrakis(triphenylphosphine)palladium, bis-(triphenylphosphine)palladium(II) chloride, tris(dibenzylideneacetone)dipalladium(0) in the presence of ligands, for example 2,2′-bis(diphenylphosphino)-1,1′-binaphthyl, 9,9-dimethyl-4,5-bis(diphenylphosphino)xanthene or 1,1′-bis-(diphenylphosphino)ferrocene.

Suitable copper salts are, for example, copper(I) iodide, copper(I) chloride, copper(I) oxide, copper(I) triflate, copper(II) acetate, copper(II) triflate, frequently in the presence of a ligand, for example diamine ligands such as N,N′-dimethylethylenediamine, N,N-dimethylethylenediamine or trans-N,N′-dimethyl-1,2-cyclohexanediamine.

Suitable nickel catalysts are, for example, nickel(II) acetylacetonate alone or in combination with the phosphor ligands mentioned above or nickel(II) acetylacetonate with imidazolium carbene ligands.

The catalyst is usually employed in a substoichiometric amount, preferably of 0.001-0.8 equivalents and particularly preferably of 0.01 to 0.5 equivalents, based on the 6-halo(thio)uracils of the formula (II) employed.

The reaction of the 6-halo(thio)uracils of the formula (II) with boron derivatives of the formula (III) is preferably carried out in a solvent selected from customary solvents which are inert under the prevailing reaction conditions. Preference is given to dioxane, tetrahydrofuran, diethyl ether, 1,2-dimethoxyethane, benzene, toluene, xylene, methanol, ethanol, isopropanol, tert-butanol, acetonitrile, propionitrile, N,N-dimethylformamide, dimethyl sulphoxide, N-methylpyrrolidone and also mixtures of these solvents with water. Organic bases such as, for example, DBU are likewise suitable.

The reaction is preferably effected in the presence of a base. Suitable bases are in particular acetates, phosphates, carbonates and bicarbonates of alkali metals or alkaline earth metals. Particular preference is given to sodium acetate, sodium phosphate, potassium phosphate, caesium carbonate, sodium carbonate, potassium carbonate, sodium hydrogencarbonate, potassium hydrogencarbonate. The inorganic bases can also be employed as aqueous solutions.

The reaction can be carried out under reduced pressure, at standard pressure or under elevated pressure and at temperatures of 0 to 200° C., preference being given to effecting the reaction at standard pressure and temperatures of 30 to 150° C., optionally under inert gas atmosphere. When carrying out the reactions, it is optionally possible to use any commercial microwave apparatus suitable for these reactions (e.g. Anton Paar Monowave 300, CEM Discover S, Biotage Initiator 60).

### Step (b)

The compounds of the formula (I-Ea) according to the invention obtained in step (a) are reacted with an oxidizing agent to give the sulphoxides of the general formula (I-Eb) according to the invention.

The reaction is carried out analogously to JP 2009/023910 or EP 0166564.

The oxidizing agents used are meta-chloroperbenzoic acid, optionally in the presence of a buffer solution and optionally in the presence of phase-transfer catalysts, rhenium salts, e.g. MeReO₃ in the presence of H₂O₂, K₂S₂O₈ in sulphuric acid or 3-40% strength aqueous solutions of hydrogen peroxide, optionally in the presence of molecular sieves or a catalyst, for example Na₂WO₄ dihydrate or sodium periodate.

The oxidation is generally conducted in a solvent selected from customary solvents which are inert under the prevailing reaction conditions.

Suitable solvents are for example dichloromethane, trichloromethane, carbon tetrachloride, 1,2-dichloroethane, chlorobenzene, dimethylformamide, methanol, ethanol, formic acid, acetic acid or propionic acid.

The reaction can be carried out at standard pressure or under elevated pressure and at temperatures from −10° C. to 80° C.; preferably, the reaction is carried out at standard pressure and temperatures of from 0° C. to 40° C.

A large number of different methods are suitable for generating enantiomerically enriched sulphoxides, as described by A. R. Maguire in ARKIVOC, 2011(i), 1-110: metal-catalysed asymmetric oxidations of thioethers, for example with titanium and vanadium as the most frequently employed catalyst sources, in the form of Ti(OⁱPr₄) and VO(acac)₂, together with a chiral ligand and an oxidizing agent such as tert-butyl hydroperoxide (TBHP), 2-phenylpropan-2-yl hydroperoxide (CHP) or hydrogen peroxide; non-metal-catalysed asymmetric oxidations employing chiral oxidizing agents or chiral catalysts; electrochemical or biological asymmetric oxidations and also kinetic resolution of sulphoxides and nucleophilic shift (according to Andersen's method).

The pure enantiomers of the compounds of the formula (I-Eb) according to the invention can be obtained from the racemate, by separating said enantiomers, for example by preparative HPLC on a chiral stationary phase.

### Process B:

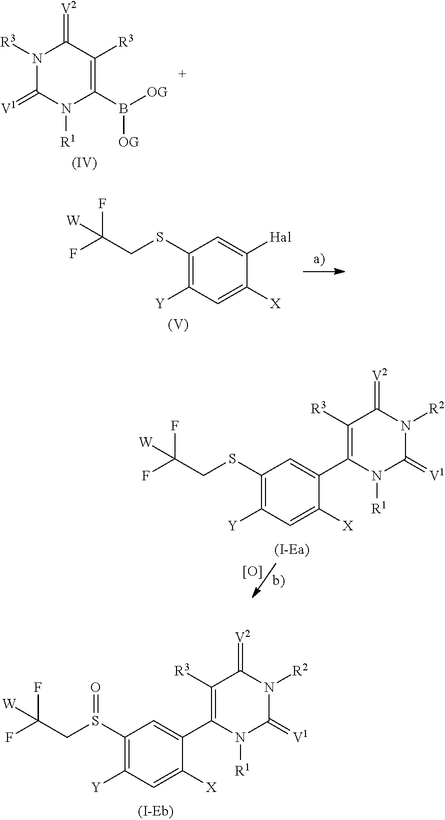

The radicals R¹, R², R³, V¹, V², G, W, X and Y have the meanings described above.

Hal represents chlorine, bromine or iodine.

### Step (a)

In a further embodiment of the invention, halosulphides of the formula (V) are reacted with uracilboric acids or 1,3,2-dioxaborolanes of the formula (IV) to give the compounds of the formula (I-Ea) according to the invention. This reaction type—reactions of boronic acids with halogenated heterocycles or halogenated aromatics—has been described, for example, in WO 2012/061557.

Corresponding reactions of 6-(tributylstannyl)pyrimidine-2,4(1H,3H)-diones with haloaromatic compounds to give 6-aryluracils are described in the literature, for example in Synthesis, 2007, 929-935.

The boric acids or 1,3,2-dioxaborolanes of the formula (IV) required as starting materials are commercially available, for example (2,6-dioxo-1,2,3,6-tetrahydropyrimidin-4-yl)boric acid (ACC Corporation, San Diego, USA), [CAS-Reg. No. 70523-23-8], or they can be synthesized by processes known from the literature as described, for example, in Tetrahedron Lett. 1978, 50, 4981-4984.

The haloaromatics of the formula (V) required as starting materials are commercially available or known from a large number of patent specifications, for example from WO 2013/157229, WO 2012/176856 or JP 2009/023910.

Suitable starting materials for the synthesis of the iodides of the general formula (V) are bromides having the same formula, which can be converted for example by halogen exchange reactions according to methods known from the literature, optionally with metal catalysis, for example analogously to Chem. Lett. 1985, 3, 411-412.

The catalysts used in the reactions of the halosulphides of the formula (V) preferably consist of salts or complexes of palladium, copper or nickel.

Suitable palladium catalysts are, for example, palladium(II) acetate, tetrakis(triphenylphosphine)palladium, bis-(triphenylphosphine)palladium(II) chloride, tris(dibenzylideneacetone)dipalladium(0) in the presence of ligands, for example 2,2′-bis(diphenylphosphino)-1,1′-binaphthyl, 9,9-dimethyl-4,5-bis(diphenylphosphino)xanthene or 1,1′-bis-(diphenylphosphino)ferrocene.

Suitable copper salts are, for example, copper(I) iodide, copper(I) chloride, copper(I) oxide, copper(I) triflate, copper(II) acetate, copper(II) triflate, frequently in the presence of a ligand, for example diamine ligands such as N,N′-dimethylethylenediamine, N,N-dimethylethylenediamine or trans-N,N′-dimethyl-1,2-cyclohexanediamine.

Suitable nickel catalysts are, for example, nickel(II) acetylacetonate alone or in combination with the phosphor ligands mentioned above or nickel(II) acetylacetonate with imidazolium carbene ligands.

The catalyst is usually employed in a substoichiometric amount, preferably of 0.001-0.8 equivalents and particularly preferably of 0.01 to 0.5 equivalents, based on the halosulphides of the formula (Va) employed.

The reaction of the halosulphides of the formula (V) with boron derivatives of the formula (IV) is preferably carried out in a solvent selected from customary solvents which are inert under the prevailing reaction conditions. Preference is given to ethers such as, for example, dioxane, tetrahydrofuran, diethyl ether or 1,2-dimethoxyethane; aromatic hydrocarbons such as, for example, benzene, toluene or xylene; aliphatic alcohols such as, for example, methanol, ethanol or isopropanol; nitriles such as, for example, acetonitrile or propionitrile; polar aprotic solvents such as N,N-dimethylformamide, dimethyl sulphoxide, N-methylpyrrolidone and also mixtures of these solvents with water.

The reaction is preferably effected in the presence of a base. Suitable bases are inorganic bases, in particular acetates, phosphates, carbonates and bicarbonates of alkali metals or alkaline earth metals. Particular preference is given to sodium acetate, sodium phosphate, potassium phosphate, caesium carbonate, sodium carbonate, potassium carbonate, sodium hydrogencarbonate, potassium hydrogencarbonate. The inorganic bases can also be employed as aqueous solutions.

The reaction can be carried out under reduced pressure, at standard pressure or under elevated pressure and at temperatures of 0 to 200° C., preference being given to effecting the reaction at standard pressure and temperatures of 30 to 150° C., optionally under inert gas atmosphere. When carrying out the reactions, it is optionally possible to use any commercial microwave apparatus suitable for these reactions (e.g. Anton Paar Monowave 300, CEM Discover S, Biotage Initiator 60).

### Step (b)

The compounds of the formula (I-Ea) according to the invention obtained in Process B by step (a) are reacted as described under Process A step (b) with an oxidizing agent to give the sulphoxides of the general formula (I-Eb) according to the invention.

### Process C

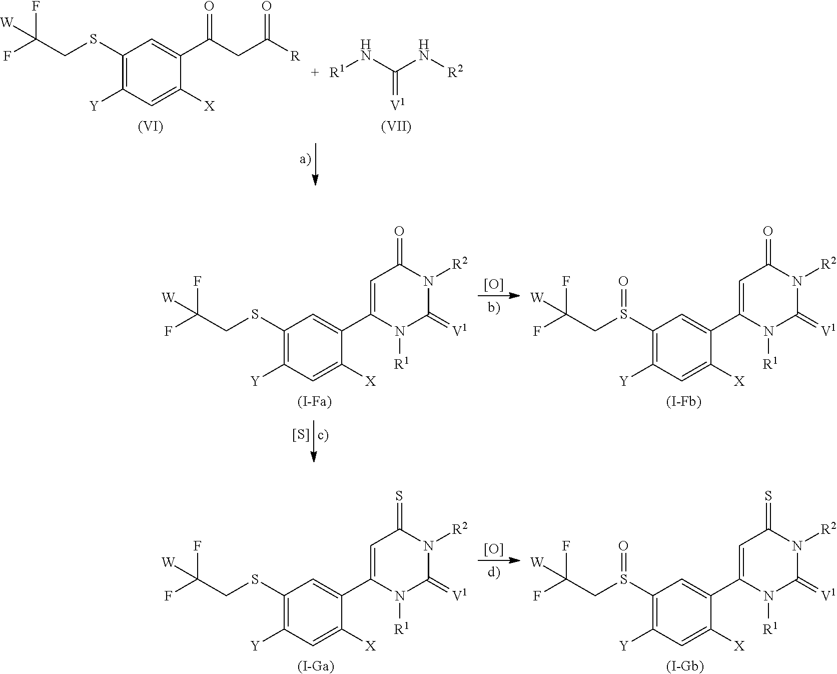

The radicals R¹, R², V¹, W, X and Y have the meanings described above.

R represents —O-alkyl (ester) or —OH (carboxylic acid).

### Step (a)

Analogously to the processes of EP 0166564, JP 59181265 or J. Med. Chem. 1986, 29, 1499-1504, 3-oxo-3-phenylpropionic acids or 3-oxo-3-phenylpropionic acid esters of the formula (VI) are reacted with (thio)ureas of the formula (VII) to give the 6-aryluracils of the formula (I-Fa) according to the invention.

The (thio)ureas of the formula (VII) required as starting materials are commercially available or can be synthesized by processes known from the literature as described, for example, in U.S. Pat. No. 4,031,218.

The oxophenylacetic acid derivatives of the formula (VI) required as starting materials can be prepared as described in Org. Proc. Res. & Dev., 1998, 2(6), 412-414, J. Org. Chem., 1979, 310 or Synthesis, 1982, 451-452.

The reaction of the oxophenylacetic acid derivatives of the formula (VI) with (thio)ureas of the formula (VII), optionally in the presence of catalytic amounts of an acid, for example sulphuric acid or aqueous HCl, is preferably carried out in a solvent selected from the customary solvents which are inert under the prevailing reaction conditions. Preference is given to ethers such as, for example, dioxane, tetrahydrofuran, diethyl ether or 1,2-dimethoxyethane, benzene, toluene or xylene, methanol, ethanol, propanol or isopropanol, dimethyl sulphoxide, N-methylpyrrolidone, acetic acid or water. It is also possible to use mixtures of these solvents.

The reactions can also be carried out in the presence of a dehydrating agent, for example magnesium sulphate, zinc chloride, phosphorus pentoxide or zeolite.

It is also possible to carry out the reactions without using a solvent.

The reaction can be effected under reduced pressure, at standard pressure or under elevated pressure and at temperatures of 0 to 200° C., preference being given to effecting the reaction at standard pressure and temperatures of 30 to 100° C.

### Step (b)

The compounds of the formula (I-Fa) according to the invention obtained in process C by step (a) are, as described under Process A step (b), reacted with an oxidizing agent to give the sulphoxides of the general formula (I-Fb) according to the invention.

### Step (c)

As described in EP 0166564, JP 59181265, J Mol. Struct., 2009, 933, 38-45, Synthesis, 1987, 256-258 or Bioorg. Med. Chem. Lett., 2010, 20, 3920-3924, the compounds of the formula (I-Fa) according to the invention are reacted with a sulphurizing agent to give the compounds of the formula (I-Ga) according to the invention, for example with phosphorus(V) sulphide or Lawesson's reagent, preferably in a solvent such as pyridine, toluene, xylene, tetrahydrofuran or dioxane.

The reaction can be carried out under reduced pressure, at standard pressure or under elevated pressure and at temperatures of 0 to 200° C., preference being given to effecting the reaction at standard pressure and temperatures of 50 to 120° C.

### Step (d)

The compounds of the formula (I-Ga) according to the invention obtained in Process C by step (c) are reacted as described under Process A step (b) with an oxidizing agent to give the sulphoxides of the general formula (I-Gb) according to the invention.

### Isomers

Depending on the nature of the substituents, the compounds of the formula (I) may be in the form of geometric and/or optically active isomers or corresponding isomer mixtures in different compositions. These stereoisomers are, for example, enantiomers, diastereomers, atropisomers or geometric isomers. The invention thus encompasses pure stereoisomers and any desired mixtures of these isomers.

### Methods and Uses

The invention also relates to methods for controlling animal pests, in which compounds of the formula (I) are allowed to act on animal pests and/or their habitat. The control of the animal pests is preferably conducted in agriculture and forestry, and in material protection. Preferably excluded from this are methods for the surgical or therapeutic treatment of the human or animal body and diagnostic methods carried out on the human or animal body.

The invention further relates to the use of the compounds of the formula (I) as pesticides, especially crop protection agents.

In the context of the present application, the term “pesticide” in each case also always comprises the term “crop protection agent”.

The compounds of the formula (I), given good plant tolerance, favourable toxicity to warm-blooded species and good environmental compatibility, are suitable for protecting plants and plant organs against biotic and abiotic stress factors, for increasing harvest yields, for improving the quality of the harvested material and for controlling animal pests, especially insects, arachnids, helminths, nematodes and molluscs, which are encountered in agriculture, in horticulture, in animal husbandry, in aquatic cultures, in forests, in gardens and leisure facilities, in the protection of stored products and of materials, and in the hygiene sector.

In the context of the present patent application, the term “hygiene” is to be understood as meaning the entirety of all measures, processes and procedures whose aim it is to prevent disorders—in particular infective diseases—and to serve keeping humans, animals and/or the environment healthy and/or to maintain cleanliness. According to the invention, this includes in particular measures for cleaning, disinfecting and sterilizing, for example, textiles or solid surfaces, mainly of glass, wood, concrete, porcelain, ceramic, plastic or else of metal(s), and keeping them clean of hygiene pests and/or their faeces. Excluded according to the invention are in this respect again processes for the surgical or therapeutic treatment of the human or animal body and diagnostic processes undertaken on the human or animal body.

The term “hygiene sector” thus includes all areas, technical fields and commercial utilizations in which such hygiene measures, processes and procedures are of importance, for example hygiene in kitchens, bakeries, airports, baths, swimming pools, shopping centres, hotels, hospitals, stables, etc.

Accordingly, the term “hygiene pest” is to be understood as meaning one or more animal pests whose presence in the hygiene sector is problematic, in particular for health reasons. Accordingly, the main aim is to minimize or prevent hygiene pests or contact therewith in the hygiene sector. This can be effected, in particular, by using a pesticide, where the agent can be employed both prophylactically and only in the case of infestation to control the pest. It is also possible to use agents which act by avoiding or reducing contact with the pest. Hygiene pests are, for example, the organisms mentioned below.

Thus, the term “hygiene protection” includes all actions which serve to maintain and/or improve such hygiene measures, processes and procedures.

The compounds of the formula (I) can preferably be used as pesticides. They are effective against normally sensitive and resistant species and against all or some stages of development. The abovementioned pests include:

pests from the phylum of the Arthropoda, especially from the class of the Arachnida, for example Acarus spp., for example Acarus siro, Aceria kuko, Aceria sheldoni, Aculops spp., Aculus spp., for example Aculus fockeui, Aculus schlechtendali, Amblyomma spp., Amphitetranychus viennensis, Argas spp., Boophilus spp., Brevipalpus spp., for example Brevipalpus phoenicis, Bryobia graminum, Bryobia praetiosa, Centruroides spp., Chorioptes spp., Dermanyssus gallinae, Dermatophagoides pteronyssinus, Dermatophagoides farinae, Dermacentor spp., Eotetranychus spp., for example Eotetranychus hicoriae, Epitrimerus pyri, Eutetranychus spp., for example Eutetranychus banksi, Eriophyes spp., for example Eriophyes pyri, Glycyphagus domesticus, Halotydeus destructor, Hemitarsonemus spp., for example Hemitarsonemus latus (=Polyphagotarsonemus latus), Hyalomma spp., Ixodes spp., Latrodectus spp., Loxosceles spp., Neutrombicula autumnalis, Nuphersa spp., Oligonychus spp., for example Oligonychus coniferarum, Oligonychus ilicis, Oligonychus indicus, Oligonychus mangiferus, Oligonychus pratensis, Oligonychus punicae, Oligonychus yothersi, Ornithodorus spp., Ornithonyssus spp., Panonychus spp., for example Panonychus citri (=Metatetranychus citri), Panonychus ulmi (=Metatetranychus ulmi), Phyllocoptruta oleivora, Platytetranychus multidigituli, Polyphagotarsonemus latus, Psoroptes spp., Rhipicephalus spp., Rhizoglyphus spp., Sarcoptes spp., Scorpio maurus, Steneotarsonemus spp., Steneotarsonemus spinki, Tarsonemus spp., for example Tarsonemus confusus, Tarsonemus pallidus, Tetranychus spp., for example Tetranychus canadensis, Tetranychus cinnabarinus, Tetranychus turkestani, Tetranychus urticae, Trombicula alfreddugesi, Vaejovis spp., Vasates lycopersici;
from the class of the Chilopoda, for example Geophilus spp., Scutigera spp.;
from the order or the class of the Collembola, for example Onychiurus armatus; Sminthurus viridis;
from the class of the Diplopoda, for example Blaniulus guttulatus;
from the class of the Insecta, for example from the order of the Blattodea, for example Blatta orientalis, Blattella asahinai, Blattella germanica, Leucophaea maderae, Panchlora spp., Parcoblatta spp., Periplaneta spp., for example Periplaneta americana, Periplaneta australasiae, Supella longipalpa;
from the order of the Coleoptera, for example Acalymma vittatum, Acanthoscelides obtectus, Adoretus spp., Agelastica alni, Agriotes spp., for example Agriotes linneatus, Agriotes mancus, Alphitobius diaperinus, Amphimallon solstitialis, Anobium punctatum, Anoplophora spp., Anthonomus spp., for example Anthonomus grandis, Anthrenus spp., Apion spp., Apogonia spp., Atomaria spp., for example Atomaria linearis, Attagenus spp., Baris caerulescens, Bruchidius obtectus, Bruchus spp., for example Bruchus pisorum, Bruchus rufimanus, Cassida spp., Cerotoma trifurcata, Ceutorrhynchus spp., for example Ceutorrhynchus assimilis, Ceutorrhynchus quadridens, Ceutorrhynchus rapae, Chaetocnema spp., for example Chaetocnema confinis, Chaetocnema denticulata, Chaetocnema ectypa, Cleonus mendicus, Conoderus spp., Cosmopolites spp., for example Cosmopolites sordidus, Costelytra zealandica, Ctenicera spp., Curculio spp., for example Curculio caryae, Curculio caryatrypes, Curculio obtusus, Curculio sayi, Cryptolestes ferrugineus, Cryptolestes pusillus, Cryptorhynchus lapathi, Cryptorhynchus mangiferae, Cylindrocopturus spp., Cylindrocopturus adspersus, Cylindrocopturus furnissi, Dermestes spp., Diabrotica spp., for example Diabrotica balteata, Diabrotica barberi, Diabrotica undecimpunctata howardi, Diabrotica undecimpunctata undecimpunctata, Diabrotica virgifera virgifera, Diabrotica virgifera zeae, Dichocrocis spp., Dicladispa armigera, Diloboderus spp., Epilachna spp., for example Epilachna borealis, Epilachna varivestis, Epitrix spp., for example Epitrix cucumeris, Epitrix fuscula, Epitrix hirtipennis, Epitrix subcrinita, Epitrix tuberis, Faustinus spp., Gibbium psylloides, Gnathocerus cornutus, Hellula undalis, Heteronychus arator, Heteronyx spp., Hylamorpha elegans, Hylotrupes bajulus, Hypera postica, Hypomeces squamosus, Hypothenemus spp., for example Hypothenemus hampei, Hypothenemus obscurus, Hypothenemus pubescens, Lachnosterna consanguinea, Lasioderma serricorne, Latheticus oryzae, Lathridius spp., Lema spp., Leptinotarsa decemlineata, Leucoptera spp., for example Leucoptera coffeella, Lissorhoptrus oryzophilus, Lixus spp., Luperomorpha xanthodera, Luperodes spp., Lyctus spp., Megascelis spp., Melanotus spp., for example Melanotus longulus oregonensis, Meligethes aeneus, Melolontha spp., for example Melolontha melolontha, Migdolus spp., Monochamus spp., Naupactus xanthographus, Necrobia spp., Niptus hololeucus, Oryctes rhinoceros, Oryzaephilus surinamensis, Oryzaphagus oryzae, Otiorhynchus spp., for example Otiorhynchus cribricollis, Otiorhynchus ligustici, Otiorhynchus ovatus, Otiorhynchus rugosostriarus, Otiorhynchus sulcatus, Oxycetonia jucunda, Phaedon cochleariae, Phyllophaga spp., Phyllophaga helleri, Phyllotreta spp., for example Phyllotreta armoraciae, Phyllotreta pusilla, Phyllotreta ramosa, Phyllotreta striolata, Popillia japonica, Premnotrypes spp., Prostephanus truncatus, Psylliodes spp., for example Psylliodes affinis, Psylliodes chrysocephala, Psylliodes punctulata, Ptinus spp., Rhizobius ventralis, Rhizopertha dominica, Sitophilus spp., for example Sitophilus granarius, Sitophilus linearis, Sitophilus oryzae, Sitophilus zeamais, Sphenophorus spp., Stegobium paniceum, Sternechus spp., for example Sternechus paludatus, Symphyletes spp., Tanymecus spp., for example Tanymecus dilaticollis, Tanymecus indicus, Tanymecus palliatus, Tenebrio molitor, Tenebrioides mauretanicus, Tribolium spp., for example Tribolium audax, Tribolium castaneum, Tribolium confusum, Trogoderma spp., Tychius spp., Xylotrechus spp., Zabrus spp., for example Zabrus tenebrioides;
from the order of the Diptera, for example Aedes spp., for example Aedes aegypti, Aedes albopictus, Aedes sticticus, Aedes vexans, Agromyza spp., for example Agromyza frontella, Agromyza parvicomis, Anastrepha spp., Anopheles spp., for example Anopheles quadrimaculatus, Anopheles gambiae, Asphondylia spp., Bactrocera spp., for example Bactrocera cucurbitae, Bactrocera dorsalis, Bactrocera oleae, Bibio hortulanus, Calliphora erythrocephala, Calliphora vicina, Ceratitis capitata, Chironomus spp., Chrysomya spp., Chrysops spp., Chrysozona pluvialis, Cochliomya spp., Contarinia spp., for example Contarinia johnsoni, Contarinia nasturtii, Contarinia pyrivora, Contarinia schulzi, Contarinia sorghicola, Contarinia tritici, Cordylobia anthropophaga, Cricotopus sylvestris, Culex spp., for example Culex pipiens, Culex quinquefasciatus, Culicoides spp., Culiseta spp., Cuterebra spp., Dacus oleae, Dasineura spp., for example Dasineura brassicae, Delia spp., for example Delia antiqua, Delia coarctata, Delia florilega, Delia platura, Delia radicum, Dermatobia hominis, Drosophila spp., for example Drosphila melanogaster, Drosophila suzukii, Echinocnemus spp., Fannia spp., Gasterophilus spp., Glossina spp., Haematopota spp., Hydrellia spp., Hydrellia griseola, Hylemya spp., Hippobosca spp., Hypoderma spp., Liriomyza spp., for example Liriomyza brassicae, Liriomyza huidobrensis, Liriomyza sativae, Lucilia spp., for example Lucilia cuprina, Lutzomyia spp., Mansonia spp., Musca spp., for example Musca domestica, Musca domestica vicina, Oestrus spp., Oscinella frit, Paratanytarsus spp., Paralauterbomiella subcincta, Pegomya spp., for example Pegomya betae, Pegomya hyoscyami, Pegomya rubivora, Phlebotomus spp., Phorbia spp., Phormia spp., Piophila casei, Prodiplosis spp., Psila rosae, Rhagoletis spp., for example Rhagoletis cingulata, Rhagoletis completa, Rhagoletis fausta, Rhagoletis indifferens, Rhagoletis mendax, Rhagoletis pomonella, Sarcophaga spp., Simulium spp., for example Simulium meridionale, Stomoxys spp., Tabanus spp., Tetanops spp., Tipula spp., for example Tipula paludosa, Tipula simplex;
from the order of the Hemiptera, for example Acizzia acaciaebaileyanae, Acizzia dodonaeae, Acizzia uncatoides, Acrida turrita, Acyrthosipon spp., for example Acyrthosiphon pisum, Acrogonia spp., Aeneolamia spp., Agonoscena spp., Aleyrodes proletella, Aleurolobus barodensis, Aleurothrixus floccosus, Allocaridara malayensis, Amrasca spp., for example Amrasca bigutulla, Amrasca devastans, Anuraphis cardui, Aonidiella spp., for example Aonidiella aurantii, Aonidiella citrina, Aonidiella inomata, Aphanostigma piri, Aphis spp., for example Aphis citricola, Aphis craccivora, Aphis fabae, Aphis forbesi, Aphis glycines, Aphis gossypii, Aphis hederae, Aphis illinoisensis, Aphis middletoni, Aphis nasturtii, Aphis nerii, Aphis pomi, Aphis spiraecola, Aphis vibumiphila, Arboridia apicalis, Arytainilla spp., Aspidiella spp., Aspidiotus spp., for example Aspidiotus nerii, Atanus spp., Aulacorthum solani, Bemisia tabaci, Blastopsylla occidentalis, Boreioglycaspis melaleucae, Brachycaudus helichrysi, Brachycolus spp., Brevicoryne brassicae, Cacopsylla spp., for example Cacopsylla pyricola, Calligypona marginata, Cameocephala fulgida, Ceratovacuna lanigera, Cercopidae, Ceroplastes spp., Chaetosiphon fragaefolii, Chionaspis tegalensis, Chlorita onukii, Chondracris rosea, Chromaphis juglandicola, Chrysomphalus ficus, Cicadulina mbila, Coccomytilus halli, Coccus spp., for example Coccus hesperidum, Coccus longulus, Coccus pseudomagnoliarum, Coccus viridis, Cryptomyzus ribis, Cryptoneossa spp., Ctenarytaina spp., Dalbulus spp., Dialeurodes citri, Diaphorina citri, Diaspis spp., Drosicha spp., Dysaphis spp., for example Dysaphis apiifolia, Dysaphis plantaginea, Dysaphis tulipae, Dysmicoccus spp., Empoasca spp., for example Empoasca abrupta, Empoasca fabae, Empoasca maligna, Empoasca solana, Empoasca stevensi, Eriosoma spp., for example Eriosoma americanum, Eriosoma lanigerum, Eriosoma pyricola, Erythroneura spp., Eucalyptolyma spp., Euphyllura spp., Euscelis bilobatus, Ferrisia spp., Geococcus coffeae, Glycaspis spp., Heteropsylla cubana, Heteropsylla spinulosa, Homalodisca coagulata, Hyalopterus arundinis, Hyalopterus pruni, Icerya spp., for example Icerya purchasi, Idiocerus spp., Idioscopus spp., Laodelphax striatellus, Lecanium spp., for example Lecanium corni (=Parthenolecanium comi), Lepidosaphes spp., for example Lepidosaphes ulmi, Lipaphis erysimi, Lycorma delicatula, Macrosiphum spp., for example Macrosiphum euphorbiae, Macrosiphum lilii, Macrosiphum rosae, Macrosteles facifrons, Mahanarva spp., Melanaphis sacchari, Metcalfiella spp., Metcalfa pruinosa, Metopolophium dirhodum, Monellia costalis, Monelliopsis pecanis, Myzus spp., for example Myzus ascalonicus, Myzus cerasi, Myzus ligustri, Myzus ornatus, Myzus persicae, Myzus nicotianae, Nasonovia ribisnigri, Nephotettix spp., for example Nephotettix cincticeps, Nephotettix nigropictus, Nilaparvata lugens, Oncometopia spp., Orthezia praelonga, Oxya chinensis, Pachypsylla spp., Parabemisia myricae, Paratrioza spp., for example Paratrioza cockerelli, Parlatoria spp., Pemphigus spp., for example Pemphigus bursarius, Pemphigus populivenae, Peregrinus maidis, Phenacoccus spp., for example Phenacoccus madeirensis, Phloeomyzus passerinii, Phorodon humuli, Phylloxera spp., for example Phylloxera devastatrix, Phylloxera notabilis, Pinnaspis aspidistrae, Planococcus spp., for example Planococcus citri, Prosopidopsylla flava, Protopulvinaria pyriformis, Pseudaulacaspis pentagona, Pseudococcus spp., for example Pseudococcus calceolariae, Pseudococcus comstocki, Pseudococcus longispinus, Pseudococcus maritimus, Pseudococcus vibumi, Psyllopsis spp., Psylla spp., for example Psylla buxi, Psylla mali, Psylla pyri, Pteromalus spp., Pyrilla spp., Quadraspidiotus spp., for example Quadraspidiotus juglansregiae, Quadraspidiotus ostreaeformis, Quadraspidiotus perniciosus, Quesada gigas, Rastrococcus spp., Rhopalosiphum spp., for example Rhopalosiphum maidis, Rhopalosiphum oxyacanthae, Rhopalosiphum padi, Rhopalosiphum rufiabdominale, Saissetia spp., for example Saissetia coffeae, Saissetia miranda, Saissetia neglecta, Saissetia oleae, Scaphoideus titanus, Schizaphis graminum, Selenaspidus articulatus, Sitobion avenae, Sogata spp., Sogatella furcifera, Sogatodes spp., Stictocephala festina, Siphoninus phillyreae, Tenalaphara malayensis, Tetragonocephela spp., Tinocallis caryaefoliae, Tomaspis spp., Toxoptera spp., for example Toxoptera aurantii, Toxoptera citricidus, Trialeurodes vaporariorum, Trioza spp., for example Trioza diospyri, Typhlocyba spp., Unaspis spp., Viteus vitifolii, Zygina spp.;
from the suborder of the Heteroptera, for example Anasa tristis, Antestiopsis spp., Boisea spp., Blissus spp., Calocoris spp., Campylomma livida, Cavelerius spp., Cimex spp., for example Cimex adjunctus, Cimex hemipterus, Cimex lectularius, Cimex pilosellus, Collaria spp., Creontiades dilutus, Dasynus piperis, Dichelops furcatus, Diconocoris hewetti, Dysdercus spp., Euschistus spp., for example Euschistus heros, Euschistus servus, Euschistus tristigmus, Euschistus variolarius, Eurygaster spp., Halyomorpha halys, Heliopeltis spp., Horcias nobilellus, Leptocorisa spp., Leptocorisa varicornis, Leptoglossus occidentalis, Leptoglossus phyllopus, Lygocoris spp., for example Lygocoris pabulinus, Lygus spp., for example Lygus elisus, Lygus hesperus, Lygus lineolaris, Macropes excavatus, Monalonion atratum, Nezara spp., for example Nezara viridula, Oebalus spp., Piesma quadrata, Piezodorus spp., for example Piezodorus guildinii, Psallus spp., Pseudacysta persea, Rhodnius spp., Sahlbergella singularis, Scaptocoris castanea, Scotinophora spp., Stephanitis nashi, Tibraca spp., Triatoma spp.;
from the order of the Hymenoptera, for example Acromyrmex spp., Athalia spp., for example Athalia rosae, Atta spp., Diprion spp., for example Diprion similis, Hoplocampa spp., for example Hoplocampa cookei, Hoplocampa testudinea, Lasius spp., Linepithema humile, Monomorium pharaonis, Sirex spp., Solenopsis invicta, Tapinoma spp., Urocerus spp., Vespa spp., for example Vespa crabro, Xeris spp.; from the order of the Isopoda, for example Armadillidium vulgare, Oniscus asellus, Porcellio scaber; from the order of the Isoptera, for example Coptotermes spp., for example Coptotermes formosanus, Cornitermes cumulans, Cryptotermes spp., Incisitermes spp., Microtermes obesi, Odontotermes spp., Reticulitermes spp., for example Reticulitermes flavipes, Reticulitermes hesperus;
from the order of the Lepidoptera, for example Achroia grisella, Acronicta major, Adoxophyes spp., for example Adoxophyes orana, Aedia leucomelas, Agrotis spp., for example Agrotis segetum, Agrotis ipsilon, Alabama spp., for example Alabama argillacea, Amyelois transitella, Anarsia spp., Anticarsia spp., for example Anticarsia gemmatalis, Argyroploce spp., Barathra brassicae, Borbo cinnara, Bucculatrix thurberiella, Bupalus piniarius, Busseola spp., Cacoecia spp., Caloptilia theivora, Capua reticulana, Carpocapsa pomonella, Carposina niponensis, Cheimatobia brumata, Chilo spp., for example Chilo plejadellus, Chilo suppressalis, Choristoneura spp., Clysia ambiguella, Cnaphalocerus spp., Cnaphalocrocis medinalis, Cnephasia spp., Conopomorpha spp., Conotrachelus spp., Copitarsia spp., Cydia spp., for example Cydia nigricana, Cydia pomonella, Dalaca noctuides, Diaphania spp., Diatraea saccharalis, Earias spp., Ecdytolopha aurantium, Elasmopalpus lignosellus, Eldana saccharina, Ephestia spp., for example Ephestia elutella, Ephestia kuehniella, Epinotia spp., Epiphyas postvittana, Etiella spp., Eulia spp., Eupoecilia ambiguella, Euproctis spp., for example Euproctis chrysorrhoea, Euxoa spp., Feltia spp., Galleria mellonella, Gracillaria spp., Grapholitha spp., for example Grapholita molesta, Grapholita prunivora, Hedylepta spp., Helicoverpa spp., for example Helicoverpa armigera, Helicoverpa zea, Heliothis spp., for example Heliothis virescens, Hofmannophila pseudospretella, Homoeosoma spp., Homona spp., Hyponomeuta padella, Kakivoria flavofasciata, Laphygma spp., Leucinodes orbonalis, Leucoptera spp., for example Leucoptera coffeella, Lithocolletis spp., for example Lithocolletis blancardella, Lithophane antennata, Lobesia spp., for example Lobesia botrana, Loxagrotis albicosta, Lymantria spp., for example Lymantria dispar, Lyonetia spp., for example Lyonetia clerkella, Malacosoma neustria, Maruca testulalis, Mamestra brassicae, Melanitis leda, Mocis spp., Monopis obviella, Mythimna separata, Nemapogon cloacellus, Nymphula spp., Oiketicus spp., Oria spp., Orthaga spp., Ostrinia spp., for example Ostrinia nubilalis, Oulema melanopus, Oulema oryzae, Panolis flammea, Parnara spp., Pectinophora spp., for example Pectinophora gossypiella, Perileucoptera spp., Phthorimaea spp., for example Phthorimaea operculella, Phyllocnistis citrella, Phyllonorycter spp., for example Phyllonorycter blancardella, Phyllonorycter crataegella, Pieris spp., for example Pieris rapae, Platynota stultana, Plodia interpunctella, Plusia spp., Plutella xylostella (=Plutella maculipennis), Prays spp., Prodenia spp., Protoparce spp., Pseudaletia spp., for example Pseudaletia unipuncta, Pseudoplusia includens, Pyrausta nubilalis, Rachiplusia nu, Schoenobius spp., for example Schoenobius bipunctifer, Scirpophaga spp., for example Scirpophaga innotata, Scotia segetum, Sesamia spp., for example Sesamia inferens, Sparganothis spp., Spodoptera spp., for example Spodoptera eradiana, Spodoptera exigua, Spodoptera frugiperda, Spodoptera praefica, Stathmopoda spp., Stomopteryx subsecivella, Synanthedon spp., Tecia solanivora, Thermesia gemmatalis, Tinea cloacella, Tinea pellionella, Tineola bisselliella, Tortrix spp., Trichophaga tapetzella, Trichoplusia spp., for example Trichoplusia ni, Tryporyza incertulas, Tuta absoluta, Virachola spp.;
from the order of the Orthoptera or Saltatoria, for example Acheta domesticus, Dichroplus spp., Gryllotalpa spp., for example Gryllotalpa gryllotalpa, Hieroglyphus spp., Locusta spp., for example Locusta migratoria, Melanoplus spp., for example Melanoplus devastator, Paratlanticus ussuriensis, Schistocerca gregaria; from the order of the Phthiraptera, for example Damalinia spp., Haematopinus spp., Linognathus spp., Pediculus spp., Phylloxera vastatrix, Phthirus pubis, Trichodectes spp.;
from the order of the Psocoptera, for example Lepinotus spp., Liposcelis spp.;
from the order of the Siphonaptera, for example Ceratophyllus spp., Ctenocephalides spp., for example Ctenocephalides canis, Ctenocephalides felis, Pulex irritans, Tunga penetrans, Xenopsylla cheopis;
from the order of the Thysanoptera, for example Anaphothrips obscurus, Baliothrips biformis, Drepanothrips reuteri, Enneothrips flavens, Frankliniella spp., for example Frankliniella fusca, Frankliniella occidentalis, Frankliniella schultzei, Frankliniella tritici, Frankliniella vaccinii, Frankliniella williamsi, Heliothrips spp., Hercinothrips femoralis, Rhipiphorothrips cruentatus, Scirtothrips spp., Taeniothrips cardamomi, Thrips spp., for example Thrips palmi, Thrips tabaci;
from the order of the Zygentoma (=Thysanura), for example Ctenolepisma spp., Lepisma saccharina, Lepismodes inquilinus, Thermobia domestica;
from the class of the Symphyla, for example Scutigerella spp., for example Scutigerella immaculata;
pests from the phylum of the Mollusca, in particular from the class of the Bivalvia, for example Dreissena spp.,
and also from the class of the Gastropoda, for example Arion spp., for example Arion ater rufus, Biomphalaria spp., Bulinus spp., Deroceras spp., for example Deroceras laeve, Galba spp., Lymnaea spp., Oncomelania spp., Pomacea spp., Succinea spp.;
animal and human parasites from the phyla of the Platyhelminthes and Nematoda, for example Aelurostrongylus spp., Amidostomum spp., Ancylostoma spp, Angiostrongylus spp., Anisakis spp., Anoplocephala spp., Ascaris spp., Ascaridia spp., Baylisascaris spp., Brugia spp., Bunostomum spp., Capillaria spp., Chabertia spp., Clonorchis spp., Cooperia spp., Crenosoma spp., Cyathostoma spp., Dicrocoelium spp., Dictyocaulus spp., Diphyllobothrium spp., Dipylidium spp., Dirofilaria spp., Dracunculus spp., Echinococcus spp., Echinostoma spp., Enterobius spp., Eucoleus spp., Fasciola spp., Fascioloides spp., Fasciolopsis spp., Filaroides spp., Gongylonema spp., Gyrodactylus spp., Habronema spp., Haemonchus spp., Heligmosomoides spp., Heterakis spp., Hymenolepis spp., Hyostrongylus spp., Litomosoides spp., Loa spp., Metastrongylus spp., Metorchis spp., Mesocestoides spp., Moniezia spp., Muellerius spp., Necator spp., Nematodirus spp., Nippostrongylus spp., Oesophagostomum spp., Ollulanus spp., Onchocerca spp, Opisthorchis spp., Oslerus spp., Ostertagia spp., Oxyuris spp., Paracapillaria spp., Parafilaria spp., Paragonimus spp., Paramphistomum spp., Paranoplocephala spp., Parascaris spp., Passalurus spp., Protostrongylus spp., Schistosoma spp., Setaria spp., Spirocerca spp., Stephanofilaria spp., Stephanurus spp., Strongyloides spp., Strongylus spp., Syngamus spp., Taenia spp., Teladorsagia spp., Thelazia spp., Toxascaris spp., Toxocara spp., Trichinella spp., Trichobilharzia spp., Trichostrongylus spp., Trichuris spp., Uncinaria spp., Wuchereria spp.;
plant pests from the phylum of the Nematoda, i.e. phytoparasitic nematodes, especially Aglenchus spp., for example Aglenchus agricola, Anguina spp., for example Anguina tritici, Aphelenchoides spp., for example Aphelenchoides arachidis, Aphelenchoides fragariae, Belonolaimus spp., for example Belonolaimus gracilis, Belonolaimus longicaudatus, Belonolaimus nortoni, Bursaphelenchus spp., for example Bursaphelenchus cocophilus, Bursaphelenchus eremus, Bursaphelenchus xylophilus, Cacopaurus spp., for example Cacopaurus pestis, Criconemella spp., for example Criconemella curvata, Criconemella onoensis, Criconemella ornata, Criconemella rusium, Criconemella xenoplax (=Mesocriconema xenoplax), Criconemoides spp., for example Criconemoides ferniae, Criconemoides onoense, Criconemoides ornatum, Ditylenchus spp., for example Ditylenchus dipsaci, Dolichodorus spp., Globodera spp., for example Globodera pallida, Globodera rostochiensis, Helicotylenchus spp., for example Helicotylenchus dihystera, Hemicriconemoides spp., Hemicycliophora spp., Heterodera spp., for example Heterodera avenae, Heterodera glycines, Heterodera schachtii, Hoplolaimus spp., Longidorus spp., for example Longidorus africanus, Meloidogyne spp., for example Meloidogyne chitwoodi, Meloidogyne fallax, Meloidogyne hapla, Meloidogyne incognita, Meloinema spp., Nacobbus spp., Neotylenchus spp., Paraphelenchus spp., Paratrichodorus spp., for example Paratrichodorus minor, Pratylenchus spp., for example Pratylenchus penetrans, Pseudohalenchus spp., Psilenchus spp., Punctodera spp., Quinisulcius spp., Radopholus spp., for example Radopholus citrophilus, Radopholus similis, Rotylenchulus spp., Rotylenchus spp., Scutellonema spp., Subanguina spp., Trichodorus spp., for example Trichodorus obtusus, Trichodorus primitivus, Tylenchorhynchus spp., for example Tylenchorhynchus annulatus, Tylenchulus spp., for example Tylenchulus semipenetrans, Xiphinema spp., for example Xiphinema index.
In addition, it is possible to control, from the sub-kingdom of the Protozoa, the order of the Coccidia, for example Eimeria spp.

### Nematodes

In the present context, the term “nematodes” comprises all species of the phylum Nematoda and here in particular species that act as parasites on plants or fungi (for example species of the order Aphelenchida, Meloidogyne, Tylenchida and others) or else on humans and animals (for example species of the orders Trichinellida, Tylenchida, Rhabditida and Spirurida) or cause damage in or on these living organisms, and also other parasitic helminths.

A nematicide in crop protection, as described herein, is capable of controlling nematodes.

The term “controlling nematodes” means killing the nematodes or preventing or impeding their development or their growth or preventing or impeding their penetration into or their sucking on plant tissue.

Here, the efficacy of the compounds is determined by comparing mortalities, gall formation, cyst formation, nematode density per volume of soil, nematode density per root, number of nematode eggs per volume of soil, mobility of the nematodes between a plant or plant part treated with the compound of the formula (I) or the treated soil and an untreated plant or plant part or the untreated soil (100%). Preferably, the reduction achieved is 25-50% in comparison to an untreated plant, plant part or the untreated soil, more preferably 51-79% and most preferably the complete kill or the complete prevention of development and growth of the nematodes by a reduction of 80 to 100%. The control of nematodes as described herein also comprises the control of proliferation of the nematodes (development of cysts and/or eggs). Compounds of the formula (I) can also be used to keep the plants or animals healthy, and they can be employed curatively, preventatively or systemically for the control of nematodes.

The person skilled in the art knows methods for determining mortalities, gall formation, cyst formation, nematode density per volume of soil, nematode density per root, number of nematode eggs per volume of soil, mobility of the nematodes.

The use of a compound of the formula (I) may keep the plant healthy and also comprises a reduction of the damage caused by nematodes and an increase of the harvest yield.

In the present context, the term “nematodes” refers to plant nematodes which comprise all nematodes which damage plants. Plant nematodes comprise phytoparasitic nematodes and soil-borne nematodes. The phytoparasitic nematodes include ectoparasites such as Xiphinema spp., Longidorus spp. and Trichodorus spp.; semiparasites such as Tylenchulus spp.; migratory endoparasites such as Pratylenchus spp., Radopholus spp. and Scutellonema spp.; non-migratory parasites such as Heterodera spp., Globodera spp. and Meloidogyne spp., and also stem and leaf endoparasites such as Ditylenchus spp., Aphelenchoides spp. and Hirschmaniella spp. Particularly damaging root-parasitic soil nematodes are, for example, cyst-forming nematodes of the genera Heterodera or Globodera, and/or root gall nematodes of the genus Meloidogyne.

Damaging species of these genera are, for example, Meloidogyne incognita, Heterodera glycines (soya bean cyst nematode), Globodera pallida and Globodera rostochiensis (yellow potato cyst nematode), these species being controlled effectively by the compounds described in the present text. However, the use of the compounds described in the present text is by no means restricted to these genera or species, but also extends in the same manner to other nematodes.

The plant nematodes include, for example, Aglenchus agricola, Anguina tritici, Aphelenchoides arachidis, Aphelenchoides fragaria, and the stem and leaf endoparasites Aphelenchoides spp., Belonolaimus gracilis, Belonolaimus longicaudatus, Belonolaimus nortoni, Bursaphelenchus cocophilus, Bursaphelenchus eremus, Bursaphelenchus xylophilus und Bursaphelenchus spp., Cacopaurus pestis, Criconemella curvata, Criconemella onoensis, Criconemella ornata, Criconemella rusium, Criconemella xenoplax (=Mesocriconema xenoplax) and Criconemella spp.,

Criconemoides femiae, Criconemoides onoense, Criconemoides ornatum and Criconemoides spp., Ditylenchus destructor, Ditylenchus dipsaci, Ditylenchus myceliophagus and also the stem and leaf endoparasites Ditylenchus spp., Dolichodorus heterocephalus, Globodera pallida (=Heterodera pallida), Globodera rostochiensis (yellow potato cyst nematode), Globodera solanacearum, Globodera tabacum, Globodera virginia and the non-migratory cyst-forming parasites Globodera spp., Helicotylenchus digonicus, Helicotylenchus dihystera, Helicotylenchus erythrine, Helicotylenchus multicinctus, Helicotylenchus nannus, Helicotylenchus pseudorobustus and Helicotylenchus spp., Hemicriconemoides, Hemicycliophora arenaria, Hemicycliophora nudata, Hemicycliophora parvana, Heterodera avenae, Heterodera cruciferae, Heterodera glycines (soya bean cyst nematode), Heterodera oryzae, Heterodera schachtii, Heterodera zeae and the non-migratory cyst-forming parasites Heterodera spp., Hirschmaniella gracilis, Hirschmaniella oryzae, Hirschmaniella spinicaudata and the stem and leaf endoparasites Hirschmaniella spp., Hoplolaimus aegyptii, Hoplolaimus californicus, Hoplolaimus columbus, Hoplolaimus galeatus, Hoplolaimus indicus, Hoplolaimus magnistylus, Hoplolaimus pararobustus, Longidorus africanus, Longidorus breviannulatus, Longidorus elongatus, Longidorus laevicapitatus, Longidorus vineacola and the ectoparasites Longidorus spp., Meloidogyne acronea, Meloidogyne africana, Meloidogyne arenaria, Meloidogyne arenaria thamesi, Meloidogyne artiella, Meloidogyne chitwoodi, Meloidogyne coffeicola, Meloidogyne ethiopica, Meloidogyne exigua, Meloidogyne fallax, Meloidogyne graminicola, Meloidogyne graminis, Meloidogyne hapla, Meloidogyne incognita, Meloidogyne incognita acrita, Meloidogyne javanica, Meloidogyne kikuyensis, Meloidogyne minor, Meloidogyne naasi, Meloidogyne paranaensis, Meloidogyne thamesi and the non-migratory parasites Meloidogyne spp., Meloinema spp., Nacobbus aberrans, Neotylenchus vigissi, Paraphelenchus pseudoparietinus, Paratrichodorus allius, Paratrichodorus lobatus, Paratrichodorus minor, Paratrichodorus nanus, Paratrichodorus porosus, Paratrichodorus teres and Paratrichodorus spp., Paratylenchus hamatus, Paratylenchus minutus, Paratylenchus projectus and Paratylenchus spp., Pratylenchus agilis, Pratylenchus alleni, Pratylenchus andinus, Pratylenchus brachyurus, Pratylenchus cerealis, Pratylenchus coffeae, Pratylenchus crenatus, Pratylenchus delattrei, Pratylenchus giibbicaudatus, Pratylenchus goodeyi, Pratylenchus hamatus, Pratylenchus hexincisus, Pratylenchus loosi, Pratylenchus neglectus, Pratylenchus penetrans, Pratylenchus pratensis, Pratylenchus scribneri, Pratylenchus teres, Pratylenchus thomei, Pratylenchus vulnus, Pratylenchus zeae and the migratory endoparasites Pratylenchus spp., Pseudohalenchus minutus, Psilenchus magnidens, Psilenchus tumidus, Punctodera chalcoensis, Quinisulcius acutus, Radopholus citrophilus, Radopholus similis, the migratory endoparasites Radopholus spp., Rotylenchulus borealis, Rotylenchulus parvus, Rotylenchulus reniformis and Rotylenchulus spp., Rotylenchus laurentinus, Rotylenchus macrodoratus, Rotylenchus robustus, Rotylenchus uniformis and Rotylenchus spp., Scutellonema brachyurum, Scutellonema bradys, Scutellonema clathricaudatum and the migratory endoparasites Scutellonema spp., Subanguina radiciola, Tetylenchus nicotianae, Trichodorus cylindricus, Trichodorus minor, Trichodorus primitivus, Trichodorus proximus, Trichodorus similis, Trichodorus sparsus and the ectoparasites Trichodorus spp., Tylenchorhynchus agri, Tylenchorhynchus brassicae, Tylenchorhynchus clarus, Tylenchorhynchus claytoni, Tylenchorhynchus digitatus, Tylenchorhynchus ebriensis, Tylenchorhynchus maximus, Tylenchorhynchus nudus, Tylenchorhynchus vulgaris and Tylenchorhynchus spp., Tylenchulus semipenetrans and the semiparasites Tylenchulus spp., Xiphinema americanum, Xiphinema brevicolle, Xiphinema dimorphicaudatum, Xiphinema index and the ectoparasites Xiphinema spp.

Nematodes for the control of which a compound of the formula (I) may be used include nematodes of the genus Meloidogyne such as the Southern root-knot nematode (Meloidogyne incognita), the Javanese root-knot nematode (Meloidogyne javanica), the Northern root-knot nematode (Meloidogyne hapla) and the peanut root-knot nematode (Meloidogyne arenaria); nematodes of the genus Ditylenchus such as the potato rot nematode (Ditylenchus destructor) and stem and bulb eelworm (Ditylenchus dipsaci); nematodes of the genus Pratylenchus such as the cob root-lesion nematode (Pratylenchus penetrans), the chrysanthemum root-lesion nematode (Pratylenchus fallax), the coffee root nematode (Pratylenchus coffeae), the tea root nematode (Pratylenchus loosi) and the walnut root-lesion nematode (Pratylenchus vulnus); nematodes of the genus Globodera such as the yellow potato cyst nematode (Globodera rostochiensis) and the white potato cyst nematode (Globodera pallida); nematodes of the genus Heterodera such as the soya bean cyst nematode (Heterodera glycines) and the beet cyst eelworm (Heterodera schachtii); nematodes of the genus Aphelenchoides such as the rice white-tip nematode (Aphelenchoides besseyi), the chrysanthemum nematode (Aphelenchoides ritzemabosi) and the strawberry nematode (Aphelenchoides fragariae); nematodes of the genus Aphelenchus such as the fungivorous nematode (Aphelenchus avenae); nematodes of the genus Radopholus, such as the burrowing nematode (Radopholus similis); nematodes of the genus Tylenchulus such as the citrus root nematode (Tylenchulus semipenetrans); nematodes of the genus Rotylenchulus such as the reniform nematode (Rotylenchulus reniformis); tree-dwelling nematodes such as the pine wood nematode (Bursaphelenchus xylophilus) and the red ring nematode (Bursaphelenchus cocophilus) and the like.

Plants for the protection of which a compound of the formula (I) can be used include plants such as cereals (for example rice, barley, wheat, rye, oats, maize and the like), beans (soya bean, azuki bean, bean, broadbean, peas, peanuts and the like), fruit trees/fruits (apples, citrus species, pears, grapevines, peaches, Japanese apricots, cherries, walnuts, almonds, bananas, strawberries and the like), vegetable species (cabbage, tomato, spinach, broccoli, lettuce, onions, spring onion, pepper and the like), root crops (carrot, potato, sweet potato, radish, lotus root, turnip and the like), plants for industrial raw materials (cotton, hemp, paper mulberry, mitsumata, rape, beet, hops, sugar cane, sugar beet, olive, rubber, palm trees, coffee, tobacco, tea and the like), cucurbits (pumpkin, cucumber, watermelon, melon and the like), meadow plants (cocksfoot, sorghum, timothy-grass, clover, alfalfa and the like), lawn grasses (mascarene grass, bentgrass and the like), spice plants etc. (lavender, rosemary, thyme, parsley, pepper, ginger and the like) and flowers (chrysanthemums, rose, orchid and the like).

The compounds of the formula (I) are particularly suitable for controlling coffee nematodes, in particular Pratylenchus brachyurus, Pratylenchus coffeae, Meloidogyne exigua, Meloidogyne incognita, Meloidogyne coffeicola, Helicotylenchus spp. and also Meloidogyne paranaensis, Rotylenchus spp., Xiphinema spp., Tylenchorhynchus spp. and Scutellonema spp.

The compounds of the formula (I) are particularly suitable for controlling potato nematodes, in particular Pratylenchus brachyurus, Pratylenchus pratensis, Pratylenchus scribneri, Pratylenchus penetrans, Pratylenchus coffeae, Ditylenchus dipsaci and of Pratylenchus alleni, Pratylenchus andinus, Pratylenchus cerealis, Pratylenchus crenatus, Pratylenchus hexincisus, Pratylenchus loosi, Pratylenchus neglectus, Pratylenchus teres, Pratylenchus thornei, Pratylenchus vulnus, Belonolaimus longicaudatus, Trichodorus cylindricus, Trichodorus primitivus, Trichodorus proximus, Trichodorus similis, Trichodorus sparsus, Paratrichodorus minor, Paratrichodorus allius, Paratrichodorus nanus, Paratrichodorus teres, Meloidogyne arenaria, Meloidogyne fallax, Meloidogyne hapla, Meloidogyne thamesi, Meloidogyne incognita, Meloidogyne chitwoodi, Meloidogyne javanica, Nacobbus aberrans, Globodera rostochiensis, Globodera pallida, Ditylenchus destructor, Radopholus similis, Rotylenchulus reniformis, Neotylenchus vigissi, Paraphelenchus pseudoparietinus, Aphelenchoides fragariae and Meloinema spp.

The compounds of the formula (I) are particularly suitable for controlling tomato nematodes, in particular Meloidogyne arenaria, Meloidogyne hapla, Meloidogyne javanica, Meloidogyne incognita, Pratylenchus penetrans and also Pratylenchus brachyurus, Pratylenchus coffeae, Pratylenchus scribneri, Pratylenchus vulnus, Paratrichodorus minor, Meloidogyne exigua, Nacobbus aberrans, Globodera solanacearum, Dolichodorus heterocephalus and Rotylenchulus reniformis.

The compounds of the formula (I) are particularly suitable for controlling cucumber plant nematodes, in particular Meloidogyne arenaria, Meloidogyne hapla, Meloidogyne javanica, Meloidogyne incognita, Rotylenchulus reniformis and Pratylenchus thornei.

The compounds of the formula (I) are particularly suitable for controlling cotton nematodes, in particular Belonolaimus longicaudatus, Meloidogyne incognita, Hoplolaimus columbus, Hoplolaimus galeatus and Rotylenchulus reniformis.

The compounds of the formula (I) are particularly suitable for controlling maize nematodes, in particular Belonolaimus longicaudatus, Paratrichodorus minor and also Pratylenchus brachyurus, Pratylenchus delattrei, Pratylenchus hexincisus, Pratylenchus penetrans, Pratylenchus zeae, (Belonolaimus gracilis), Belonolaimus nortoni, Longidorus breviannulatus, Meloidogyne arenaria, Meloidogyne arenaria thamesi, Meloidogyne graminis, Meloidogyne incognita, Meloidogyne incognita acrita, Meloidogyne javanica, Meloidogyne naasi, Heterodera avenae, Heterodera oryzae, Heterodera zeae, Punctodera chalcoensis, Ditylenchus dipsaci, Hoplolaimus aegyptii, Hoplolaimus magnistylus, Hoplolaimus galeatus, Hoplolaimus indicus, Helicotylenchus digonicus, Helicotylenchus dihystera, Helicotylenchus pseudorobustus, Xiphinema americanum, Dolichodorus heterocephalus, Criconemella ornata, Criconemella onoensis, Radopholus similis, Rotylenchulus borealis, Rotylenchulus parvus, Tylenchorhynchus agri, Tylenchorhynchus clarus, Tylenchorhynchus claytoni, Tylenchorhynchus maximus, Tylenchorhynchus nudus, Tylenchorhynchus vulgaris, Quinisulcius acutus, Paratylenchus minutus, Hemicycliophora parvana, Aglenchus agricola, Anguina tritici, Aphelenchoides arachidis, Scutellonema brachyurum and Subanguina radiciola.

The compounds of the formula (I) are particularly suitable for controlling soya bean nematodes, in particular Pratylenchus brachyurus, Pratylenchus pratensis, Pratylenchus penetrans, Pratylenchus scribneri, Belonolaimus longicaudatus, Heterodera glycines, Hoplolaimus columbus and also Pratylenchus coffeae, Pratylenchus hexincisus, Pratylenchus neglectus, Pratylenchus crenatus, Pratylenchus alleni, Pratylenchus agilis, Pratylenchus zeae, Pratylenchus vulnus, (Belonolaimus gracilis), Meloidogyne arenaria, Meloidogyne incognita, Meloidogyne javanica, Meloidogyne hapla, Hoplolaimus columbus, Hoplolaimus galeatus and Rotylenchulus reniformis.

The compounds of the formula (I) are particularly suitable for controlling tobacco nematodes, in particular Meloidogyne incognita, Meloidogyne javanica and also Pratylenchus brachyurus, Pratylenchus pratensis, Pratylenchus hexincisus, Pratylenchus penetrans, Pratylenchus neglectus, Pratylenchus crenatus, Pratylenchus thornei, Pratylenchus vulnus, Pratylenchus zeae, Longidorus elongatu, Paratrichodorus lobatus, Trichodorus spp., Meloidogyne arenaria, Meloidogyne hapla, Globodera tabacum, Globodera solanacearum, Globodera virginiae, Ditylenchus dipsaci, Rotylenchus spp., Helicotylenchus spp., Xiphinema americanum, Criconemella spp., Rotylenchulus reniformis, Tylenchorhynchus claytoni, Paratylenchus spp. and Tetylenchus nicotianae.

The compounds of the formula (I) are particularly suitable for controlling citrus nematodes, in particular Pratylenchus coffeae and also Pratylenchus brachyurus, Pratylenchus vulnus, Belonolaimus longicaudatus, Paratrichodorus minor, Paratrichodorus porosus, Trichodorus, Meloidogyne incognita, Meloidogyne incognita acrita, Meloidogyne javanica, Rotylenchus macrodoratus, Xiphinema americanum, Xiphinema brevicolle, Xiphinema index, Criconemella spp., Hemicriconemoides, Radopholus similis and Radopholus citrophilus, Hemicycliophora arenaria, Hemicycliophora nudata and Tylenchulus semipenetrans.

The compounds of the formula (I) are particularly suitable for controlling banana nematodes, in particular Pratylenchus coffeae, Radopholus similis and also Pratylenchus giibbicaudatus, Pratylenchus loosi, Meloidogyne spp., Helicotylenchus multicinctus, Helicotylenchus dihystera and Rotylenchulus spp.

The compounds of the formula (I) are particularly suitable for controlling pineapple nematodes, in particular Pratylenchus zeae, Pratylenchus pratensis, Pratylenchus brachyurus, Pratylenchus goodeyi., Meloidogyne spp., Rotylenchulus reniformis and of Longidorus elongatus, Longidorus laevicapitatus, Trichodorus primitivus, Trichodorus minor, Heterodera spp., Ditylenchus myceliophagus, Hoplolaimus califomicus, Hoplolaimus pararobustus, Hoplolaimus indicus, Helicotylenchus dihystera, Helicotylenchus nannus, Helicotylenchus multicinctus, Helicotylenchus erythrine, Xiphinema dimorphicaudatum, Radopholus similis, Tylenchorhynchus digitatus, Tylenchorhynchus ebriensis, Paratylenchus minutus, Scutellonema clathricaudatum, Scutellonema bradys, Psilenchus tumidus, Psilenchus magnidens, Pseudohalenchus minutus, Criconemoides femiae, Criconemoides onoense and Criconemoides ornatum.

The compounds of the formula (I) are particularly suitable for controlling grapevine nematodes, in particular Pratylenchus vulnus, Meloidogyne arenaria, Meloidogyne incognita, Meloidogyne javanica, Xiphinema americanum, Xiphinema index and of Pratylenchus pratensis, Pratylenchus scribneri, Pratylenchus neglectus, Pratylenchus brachyurus, Pratylenchus thomei and Tylenchulus semipenetrans.

The compounds of the formula (I) are particularly suitable for controlling nematodes in tree crops—pome fruit, in particular Pratylenchus penetrans and of Pratylenchus vulnus, Longidorus elongatus, Meloidogyne incognita and Meloidogyne hapla.

The compounds of the formula (I) are particularly suitable for controlling nematodes in tree crops—stone fruit, in particular Pratylenchus penetrans, Pratylenchus vulnus, Meloidogyne arenaria, Meloidogyne hapla, Meloidogyne javanica, Meloidogyne incognita, Criconemella xenoplax and of Pratylenchus brachyurus, Pratylenchus coffeae, Pratylenchus scribneri, Pratylenchus zeae, Belonolaimus longicaudatus, Helicotylenchus dihystera, Xiphinema americanum, Criconemella curvata, Tylenchorhynchus claytoni, Paratylenchus hamatus, Paratylenchus projectus, Scutellonema brachyurum and Hoplolaimus galeatus.

The compounds of the formula (I) are particularly suitable for controlling nematodes in tree crops, sugar cane and rice, in particular Trichodorus spp., Criconemella spp. and of Pratylenchus spp., Paratrichodorus spp., Meloidogyne spp., Helicotylenchus spp., Tylenchorhynchus spp., Aphelenchoides spp., Heterodera spp, Xiphinema spp. and Cacopaurus pestis.

In the present context, the term “nematodes” also refers to nematodes damaging humans or animals.

Specific nematode species harmful to humans or to animals are:

Trichinellida, for example: Trichuris spp., Capillaria spp., Paracapillaria spp., Eucoleus spp., Trichomosoides spp., Trichinella spp.
From the order of the Tylenchida, for example: Micronema spp., Strongyloides spp.
From the order of the Rhabditida, for example: Strongylus spp., Triodontophorus spp., Oesophagodontus spp., Trichonema spp., Gyalocephalus spp., Cylindropharynx spp., Poteriostomum spp., Cyclococercus spp., Cylicostephanus spp., Oesophagostomum spp., Chabertia spp., Stephanurus spp., Ancylostoma spp., Uncinaria spp., Necator spp., Bunostomum spp., Globocephalus spp., Syngamus spp., Cyathostoma spp., Metastrongylus spp., Dictyocaulus spp., Muellerius spp., Protostrongylus spp., Neostrongylus spp., Cystocaulus spp., Pneumostrongylus spp., Spicocaulus spp., Elaphostrongylus spp., Parelaphostrongylus spp., Crenosoma spp., Paracrenosoma spp., Oslerus spp., Angiostrongylus spp., Aelurostrongylus spp., Filaroides spp., Parafilaroides spp., Trichostrongylus spp., Haemonchus spp., Ostertagia spp., Teladorsagia spp., Marshallagia spp., Cooperia spp., Nippostrongylus spp., Heligmosomoides spp., Nematodirus spp., Hyostrongylus spp., Obeliscoides spp., Amidostomum spp., Ollulanus spp.

From the order of the Spirurida, for example: Oxyuris spp., Enterobius spp., Passalurus spp., Syphacia spp., Aspiculuris spp., Heterakis spp.; Ascaris spp., Toxascaris spp., Toxocara spp., Baylisascaris spp., Parascaris spp., Anisakis spp., Ascaridia spp.; Gnathostoma spp., Physaloptera spp., Thelazia spp., Gongylonema spp., Habronema spp., Parabronema spp., Draschia spp., Dracunculus spp.; Stephanofilaria spp., Parafilaria spp., Setaria spp., Loa spp., Dirofilaria spp., Litomosoides spp., Brugia spp., Wuchereria spp., Onchocerca spp., Spirocerca spp.;

Many known nematicides also act against other parasitic helminths and are therefore used for controlling worms—not necessarily belonging to the group Nematoda—which are parasites in humans and animals. The present invention also relates to the use of the compounds of the formula (I) as anthelmintic medicaments. The pathogenic endoparasitic helminths include Platyhelminthes (e.g. Monogenea, cestodes and trematodes), Acanthocephala and Pentastoma. The following helminths may be mentioned as being preferred:

Monogenea: for example: Gyrodactylus spp., Dactylogyrus spp., Polystoma spp.

Cestodes: from the order of the Pseudophyllidea, for example: Diphyllobothrium spp., Spirometra spp., Schistocephalus spp., Ligula spp., Bothridium spp., Diplogonoporus spp.

From the order of the Cyclophyllida, for example: Mesocestoides spp., Anoplocephala spp., Paranoplocephala spp., Moniezia spp., Thysanosoma spp., Thysaniezia spp., Avitellina spp., Stilesia spp., Cittotaenia spp., Andyra spp., Bertiella spp., Taenia spp., Echinococcus spp., Hydatigera spp., Davainea spp., Raillietina spp., Hymenolepis spp., Echinolepis spp., Echinocotyle spp., Diorchis spp., Dipylidium spp., Joyeuxiella spp., Diplopylidium spp.

Trematodes: from the class of the Digenea, for example: Diplostomum spp., Posthodiplostomum spp., Schistosoma spp., Trichobilharzia spp., Ornithobilharzia spp., Austrobilharzia spp., Gigantobilharzia spp., Leucochloridium spp., Brachylaima spp., Echinostoma spp., Echinoparyphium spp., Echinochasmus spp., Hypoderaeum spp., Fasciola spp., Fasciolides spp., Fasciolopsis spp., Cyclocoelum spp., Typhlocoelum spp., Paramphistomum spp., Calicophoron spp., Cotylophoron spp., Gigantocotyle spp., Fischoederius spp., Gastrothylacus spp., Notocotylus spp., Catatropis spp., Plagiorchis spp., Prosthogonimus spp., Dicrocoelium spp., Eurytrema spp., Troglotrema spp., Paragonimus spp., Collyriclum spp., Nanophyetus spp., Opisthorchis spp., Clonorchis spp., Metorchis spp., Heterophyes spp., Metagonimus spp.

Acanthocephala: from the order of the Oligacanthorhynchida, for example: Macracanthorhynchus spp., Prosthenorchis spp.; from the order of the Polymorphida, for example: Filicollis spp.; from the order of the Moniliformida, for example: Moniliformis spp.

From the order of the Echinorhynchida, for example, Acanthocephalus spp., Echinorhynchus spp., Leptorhynchoides spp.

Pentastoma: from the order of the Porocephalida, for example, Linguatula spp.

In the veterinary field and in animal keeping, the administration of the compounds of the formula (I) is carried out in a known manner, directly or enterally, parenterally, dermally or nasally in the form of suitable use forms. Administration may be prophylactic or therapeutic.

The compounds of the formula (I) can optionally, at certain concentrations or application rates, also be used as herbicides, safeners, growth regulators or agents to improve plant properties, as microbicides or gametocides, for example as fungicides, antimycotics, bactericides, virucides (including agents against viroids) or as agents against MLO (mycoplasma-like organisms) and RLO (rickettsia-like organisms). If appropriate, they can also be used as intermediates or precursors for the synthesis of other active compounds.

### Formulations

The present invention further relates to formulations and use forms prepared therefrom as pesticides, for example drench, drip and spray liquors, comprising at least one compound of the formula (I). In some cases, the use forms comprise further pesticides and/or adjuvants which improve the action, such as penetrants, e.g. vegetable oils, for example rapeseed oil, sunflower oil, mineral oils, for example paraffin oils, alkyl esters of vegetable fatty acids, for example rapeseed oil methyl ester or soya oil methyl ester, or alkanol alkoxylates and/or spreaders, for example alkylsiloxanes and/or salts, for example organic or inorganic ammonium or phosphonium salts, for example ammonium sulphate or diammonium hydrogenphosphate and/or retention promoters, for example dioctyl sulphosuccinate or hydroxypropyl guar polymers and/or humectants, for example glycerol and/or fertilizers, for example ammonium-, potassium- or phosphorus-containing fertilizers.

Customary formulations are, for example, water-soluble liquids (SL), emulsion concentrates (EC), emulsions in water (EW), suspension concentrates (SC, SE, FS, OD), water-dispersible granules (WG), granules (GR) and capsule concentrates (CS); these and further possible formulation types are described, for example, by Crop Life International and in Pesticide Specifications, Manual on development and use of FAO and WHO specifications for pesticides, FAO Plant Production and Protection Papers—173, prepared by the FAO/WHO Joint Meeting on Pesticide Specifications, 2004, ISBN: 9251048576. The formulations, in addition to one or more compounds of the formula (I), optionally comprise further agrochemical active ingredients.

These are preferably formulations or use forms which comprise auxiliaries, for example extenders, solvents, spontaneity promoters, carriers, emulsifiers, dispersants, frost protectants, biocides, thickeners and/or further auxiliaries, for example adjuvants. An adjuvant in this context is a component which enhances the biological effect of the formulation, without the component itself having any biological effect. Examples of adjuvants are agents which promote retention, spreading, attachment to the leaf surface or penetration.

These formulations are prepared in a known way, for example by mixing the compounds of the formula (I) with auxiliaries, for example extenders, solvents and/or solid carriers and/or other auxiliaries, for example surfactants. The formulations are produced either in suitable facilities or else before or during application.

The auxiliaries used may be substances suitable for imparting special properties, such as certain physical, technical and/or biological properties, to the formulation of the compounds of the formula (I), or to the use forms prepared from these formulations (for example ready-to-use pesticides such as spray liquors or seed dressing products).

Suitable extenders are, for example, water, polar and nonpolar organic chemical liquids, for example from the classes of the aromatic and non-aromatic hydrocarbons (such as paraffins, alkylbenzenes, alkylnaphthalenes, chlorobenzenes), the alcohols and polyols (which, if appropriate, may also be substituted, etherified and/or esterified), the ketones (such as acetone, cyclohexanone), esters (including fats and oils) and (poly)ethers, the unsubstituted and substituted amines, amides, lactams (such as N-alkylpyrrolidones) and lactones, the sulphones and sulphoxides (such as dimethyl sulphoxide).

If the extender utilized is water, it is also possible to use, for example, organic solvents as auxiliary solvents. Useful liquid solvents are essentially: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example petroleum fractions, mineral and vegetable oils, alcohols such as butanol or glycol and their ethers and esters, ketones such as acetone, methyl ethyl ketone, methyl isobutyl ketone or cyclohexanone, strongly polar solvents such as dimethylformamide and dimethyl sulphoxide, and also water.

In principle, it is possible to use all suitable solvents. Examples of suitable solvents are aromatic hydrocarbons, such as xylene, toluene or alkylnaphthalenes, chlorinated aromatic or chlorinated aliphatic hydrocarbons, such as chlorobenzene, chloroethylene or methylene chloride, aliphatic hydrocarbons, such as cyclohexane, paraffins, petroleum fractions, mineral and vegetable oils, alcohols, such as methanol, ethanol, isopropanol, butanol or glycol and their ethers and esters, ketones such as acetone, methyl ethyl ketone, methyl isobutyl ketone or cyclohexanone, strongly polar solvents, such as dimethyl sulphoxide, and also water.

In principle, it is possible to use all suitable carriers. Useful carriers especially include: for example ammonium salts and ground natural minerals such as kaolins, clays, talc, chalk, quartz, attapulgite, montmorillonite or diatomaceous earth, and ground synthetic minerals such as finely divided silica, alumina and natural or synthetic silicates, resins, waxes and/or solid fertilizers. Mixtures of such carriers can likewise be used. Useful carriers for granules include: for example crushed and fractionated natural rocks such as calcite, marble, pumice, sepiolite, dolomite, and synthetic granules of inorganic and organic meals, and also granules of organic material such as sawdust, paper, coconut shells, corn cobs and tobacco stalks.

Liquefied gaseous extenders or solvents can also be used. Especially suitable are those extenders or carriers which are gaseous at standard temperature and under standard pressure, for example aerosol propellants such as halohydrocarbons, and also butane, propane, nitrogen and carbon dioxide.

Examples of emulsifiers and/or foam formers, dispersants or wetting agents with ionic or nonionic properties, or mixtures of these surfactants, include salts of polyacrylic acid, salts of lignosulphonic acid, salts of phenolsulphonic acid or naphthalenesulphonic acid, polycondensates of ethylene oxide with fatty alcohols or with fatty acids or with fatty amines, with substituted phenols (preferably alkylphenols or arylphenols), salts of sulphosuccinic esters, taurine derivatives (preferably alkyl taurates), phosphoric esters of polyethoxylated alcohols or phenols, fatty acid esters of polyols, and derivatives of the compounds containing sulphates, sulphonates and phosphates, for example alkylaryl polyglycol ethers, alkylsulphonates, alkyl sulphates, arylsulphonates, protein hydrolysates, lignosulphite waste liquors and methylcellulose. The presence of a surfactant is advantageous if one of the compounds of the formula (I) and/or one of the inert carriers is insoluble in water and when the application takes place in water.

Further auxiliaries which may be present in the formulations and the use forms derived therefrom include dyes such as inorganic pigments, for example iron oxide, titanium oxide and Prussian Blue, and organic dyes such as alizarin dyes, azo dyes and metal phthalocyanine dyes, and nutrients and trace nutrients such as salts of iron, manganese, boron, copper, cobalt, molybdenum and zinc.

Additional components may be stabilizers, such as cold stabilizers, preservatives, antioxidants, light stabilizers, or other agents which improve the chemical and/or physical stability. Foam generators or antifoams may also be present.

In addition, the formulations and the use forms derived therefrom may also comprise, as additional auxiliaries, stickers such as carboxymethylcellulose and natural and synthetic polymers in the form of powders, granules or latices, such as gum arabic, polyvinyl alcohol and polyvinyl acetate, or else natural phospholipids such as cephalins and lecithins and synthetic phospholipids. Further possible auxiliaries are mineral and vegetable oils.

Optionally, further auxiliaries may be present in the formulations and the use forms derived therefrom. Examples of such additives include fragrances, protective colloids, binders, adhesives, thickeners, thixotropic agents, penetrants, retention promoters, stabilizers, sequestrants, complexing agents, humectants, spreaders. In general, the compounds of the formula (I) can be combined with any solid or liquid additive commonly used for formulation purposes.

Useful retention promoters include all those substances which reduce the dynamic surface tension, for example dioctyl sulphosuccinate, or increase the viscoelasticity, for example hydroxypropylguar polymers.

Suitable penetrants in the present context are all those substances which are usually used for improving the penetration of agrochemical active compounds into plants. Penetrants are defined in this context by their ability to penetrate from the (generally aqueous) application liquor and/or from the spray coating into the cuticle of the plant and hence increase the mobility of the active compounds in the cuticle. The method described in the literature (Baur et al., 1997, Pesticide Science 51, 131-152) can be used for determining this property. Examples include alcohol alkoxylates such as coconut fatty ethoxylate (10) or isotridecyl ethoxylate (12), fatty acid esters, for example rapeseed oil methyl ester or soya oil methyl ester, fatty amine alkoxylates, for example tallowamine ethoxylate (15), or ammonium and/or phosphonium salts, for example ammonium sulphate or diammonium hydrogenphosphate.

The formulations preferably comprise between 0.00000001% and 98% by weight of the compound of the formula (I) or, with particular preference, between 0.01% and 95% by weight of the compound of the formula (I), more preferably between 0.5% and 90% by weight of the compound of the formula (I), based on the weight of the formulation.

The content of the compound of the formula (I) in the use forms prepared from the formulations (in particular pesticides) may vary within wide ranges. The concentration of the compound of the formula (I) in the use forms may typically be between 0.00000001% and 95% by weight of the compound of the formula (I), preferably between 0.00001% and 1% by weight, based on the weight of the use form. The application is accomplished in a customary manner appropriate for the use forms.

### Mixtures

The compounds of the formula (I) may also be employed as a mixture with one or more suitable fungicides, bactericides, acaricides, molluscicides, nematicides, insecticides, microbiologicals, beneficial species, herbicides, fertilizers, bird repellents, phytotonics, sterilants, safeners, semiochemicals and/or plant growth regulators, in order thus, for example, to broaden the spectrum of action, to prolong the duration of action, to increase the rate of action, to prevent repulsion or prevent evolution of resistance. In addition, active compound combinations of this kind can improve plant growth and/or tolerance to abiotic factors, for example high or low temperatures, to drought or to elevated water content or soil salinity. It is also possible to improve flowering and fruiting performance, optimize germination capacity and root development, facilitate harvesting and improve yields, influence maturation, improve the quality and/or the nutritional value of the harvested products, prolong storage life and/or improve the processability of the harvested products.

Furthermore, the compounds of the formula (I) can be present in a mixture with other active compounds or semiochemicals such as attractants and/or bird repellents and/or plant activators and/or growth regulators and/or fertilizers. Likewise, the compounds of the formula (I) can be used in mixtures with agents to improve plant properties, for example growth, yield and quality of the harvested material.

In a particular embodiment of the invention, the compounds of the formula (I) are in the form of formulations or the use forms prepared from these formulations in a mixture with further compounds, preferably those as described below.

If one of the compounds mentioned below can occur in various tautomeric forms, these forms are also included even if not explicitly mentioned in each case.

### Insecticides/Acaricides/Nematicides

The active compounds identified here by their common names are known and are described, for example, in the pesticide handbook (“The Pesticide Manual” 16th Ed., British Crop Protection Council 2012) or can be found on the Internet (e.g. http://www.alanwood.net/pesticides).

All insecticidal/acaricidal/nematicidal mixing partners of classes (1) to (29) mentioned may, their functional groups permitting, optionally form salts with suitable bases or acids. All mixing partners of classes (1) to (29) mentioned may, if applicable, include tautomeric forms.

(1) Acetylcholinesterase (AChE) inhibitors, such as, for example, carbamates, for example alanycarb, aldicarb, bendiocarb, benfuracarb, butocarboxim, butoxycarboxim, carbaryl, carbofuran, carbosulphan, ethiofencarb, fenobucarb, formetanate, furathiocarb, isoprocarb, methiocarb, methomyl, metolcarb, oxamyl, pirimicarb, propoxur, thiodicarb, thiofanox, triazamate, trimethacarb, XMC and xylylcarb; or organophosphates, for example acephate, azamethiphos, azinphos-ethyl, azinphos-methyl, cadusafos, chlorethoxyfos, chlorfenvinphos, chlormephos, chlorpyrifos, chlorpyrifos-methyl, coumaphos, cyanophos, demeton-S-methyl, diazinon, dichlorvos/DDVP, dicrotophos, dimethoate, dimethylvinphos, disulphoton, EPN, ethion, ethoprophos, famphur, fenamiphos, fenitrothion, fenthion, fosthiazate, heptenophos, imicyafos, isofenphos, isopropyl O-(methoxyaminothiophosphoryl) salicylate, isoxathion, malathion, mecarbam, methamidophos, methidathion, mevinphos, monocrotophos, naled, omethoate, oxydemeton-methyl, parathion, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulphotep, tebupirimfos, temephos, terbufos, tetrachlorvinphos, thiometon, triazophos, triclorfon and vamidothion.

(2) GABA-gated chloride channel antagonists, for example cyclodiene-organochlorines, e.g. chlordane and endosulphan or phenylpyrazoles (fiproles), e.g. ethiprole and fipronil.

(3) Sodium channel modulators/voltage-gated sodium channel blockers, for example pyrethroids, e.g. acrinathrin, allethrin, d-cis-trans allethrin, d-trans allethrin, bifenthrin, bioallethrin, bioallethrin S-cyclopentenyl isomer, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin [(1R)-trans isomers], deltamethrin, empenthrin [(EZ)-(1R) isomers], esfenvalerate, etofenprox, fenpropathrin, fenvalerate, flucythrinate, flumethrin, tau-fluvalinate, halfenprox, imiprothrin, kadethrin, permethrin, phenothrin [(1R)-trans isomer], prallethrin, pyrethrins (pyrethrum), resmethrin, silafluofen, tefluthrin, tetramethrin, tetramethrin [(1R) isomers], tralomethrin and transfluthrin or DDT or methoxychlor.

(4) Nicotinergic acetylcholine receptor (nAChR) agonists, for example neonicotinoids, e.g. acetamiprid, clothianidin, dinotefuran, imidacloprid, nitenpyram, thiacloprid and thiamethoxam or nicotine or sulphoxaflor.

(5) Allosteric activators of the nicotinergic acetylcholine receptor (nAChR), for example spinosyns, e.g. spinetoram and spinosad.

(6) Chloride channel activators, for example avermectins/milbemycins, e.g. abamectin, emamectin benzoate, lepimectin and milbemectin.

(7) Juvenile hormone imitators, for example, juvenile hormone analogues, e.g. hydroprene, kinoprene and methoprene or fenoxycarb or pyriproxyfen.

(8) Active compounds with unknown or nonspecific mechanisms of action, for example alkyl halides, e.g. methyl bromide and other alkyl halides; or chloropicrine or sulphuryl fluoride or borax or tartar emetic.

(9) Selective antifeedants, e.g. pymetrozine or flonicamid.

(10) Mite growth inhibitors, e.g. clofentezine, hexythiazox and diflovidazin or etoxazole.

(11) Microbial disruptors of the insect gut membrane, e.g. Bacillus thuringiensis subspecies israelensis, Bacillus sphaericus, Bacillus thuringiensis subspecies aizawai, Bacillus thuringiensis subspecies kurstaki, Bacillus thuringiensis subspecies tenebrionis, and BT plant proteins: Cry1Ab, Cry1Ac, Cry1Fa, Cry2Ab, mCry3A, Cry3Ab, Cry3Bb, Cry34/35Ab1.

(12) Oxidative phosphorylation inhibitors, ATP disruptors, for example diafenthiuron or organotin compounds, e.g. azocyclotin, cyhexatin and fenbutatin oxide or propargite or tetradifon.

(13) Oxidative phosphorylation decouplers that interrupt the H proton gradient, for example chlorfenapyr, DNOC and sulphluramid.

(14) Nicotinergic acetylcholine receptor antagonists, for example bensultap, cartap hydrochloride, thiocyclam, and thiosultap-sodium.

(15) Chitin biosynthesis inhibitors, type 0, for example bistrifluron, chlorfluazuron, diflubenzuron, flucycloxuron, flufenoxuron, hexaflumuron, lufenuron, novaluron, noviflumuron, teflubenzuron and triflumuron.

(16) Chitin biosynthesis inhibitors, type 1, for example buprofezin.

(17) Moulting inhibitors (in particular for Diptera, i.e. dipterans) such as, for example, cyromazine.

(18) Ecdysone receptor agonists, for example chromafenozide, halofenozide, methoxyfenozide and tebufenozide.

(19) Octopaminergic agonists, for example amitraz.

(20) Complex-III electron transport inhibitors, for example hydramethylnon or acequinocyl or fluacrypyrim.

(21) Complex-I electron transport inhibitors, for example METI acaricides, e.g. fenazaquin, fenpyroximate, pyrimidifen, pyridaben, tebufenpyrad and tolfenpyrad or rotenone (Derris).

(22) Voltage-gated sodium channel blockers, for example indoxacarb or metaflumizone.

(23) Inhibitors of acetyl-CoA carboxylase, for example tetronic and tetramic acid derivatives, e.g. spirodiclofen, spiromesifen and spirotetramat.

(24) Complex-IV electron transport inhibitors, for example phosphines, e.g. aluminium phosphide, calcium phosphide, phosphine and zinc phosphide or cyanide.

(25) Complex-II electron transport inhibitors, for example cyenopyrafen and cyflumetofen.

(28) Ryanodine receptor effectors, for example diamides, e.g. chlorantraniliprole, cyantraniliprole and flubendiamide,

(29) further active compounds, for example afidopyropen, azadirachtin, benclothiaz, benzoximate, bifenazate, bromopropylate, chinomethionat, cryolite,

dicofol, diflovidazin, fluensulphone, flometoquin, flufenerim, flufenoxystrobin, flufiprole, fluopyram, flupyradifurone, fufenozide, heptafluthrin, imidaclothiz, iprodione, meperfluthrin, paichongding, pyflubumide, pyrifluquinazon, pyriminostrobin, tetramethylfluthrin and iodomethane; and also preparations based on Bacillus firmus (1-1582, BioNeem, Votivo), and also the following compounds: 3-bromo-N-{2-bromo-4-chloro-6-[(-cyclopropylethyl)carbamoyl]phenyl}-1-(3-chloropyridin-2-yl)-H-pyrazole-5-carboxamide (known from WO2005/077934) and 1-{2-fluoro-4-methyl-5-[(2,2,2-trifluoroethyl)sulphinyl]phenyl}-3-(trifluoromethyl)-1H-1,2,4-triazole-5-amine (known from WO2006/043635), {1′-[(2E)-3-(4-chlorophenyl)prop-2-en-1-yl]-5-fluorospiro [indole-3,4′-piperidin]-1 (2H)-yl}(2-chloropyridin-4-yl)methanone (known from WO2003/106457), 2-chloro-N-[2-{1-[(2E)-3-(4-chlorophenyl)prop-2-en-1-yl]piperidin-4-yl}-4-(trifluoromethyl)phenyl]isonicotinamide (known from WO2006/003494), 3-(2,5-dimethylphenyl)-4-hydroxy-8-methoxy-1,8-diazaspiro[4.5]dec-3-en-2-one (known from WO2009/049851), 3-(2,5-dimethylphenyl)-8-methoxy-2-oxo-1,8-diazaspiro[4.5]dec-3-en-4-yl-ethylcarbonate (known from WO2009/049851), 4-(but-2-yn-1-yloxy)-6-(3,5-dimethylpiperidin-1-yl)-5-fluoropyrimidine (known from WO2004/099160), 4-(but-2-yn-1-yloxy)-6-(3-chlorophenyl)pyrimidine (known from WO2003/076415), PF1364 (CAS Reg. No. 1204776-60-2), 4-[5-(3,5-dichlorophenyl)-5-(trifluoromethyl)-4,5-dihydro-1,2-oxazol-3-yl]-2-methyl-N-{2-oxo-2-[(2,2,2-trifluoroethyl)amino]ethyl}benzamide (known from WO2005/085216), 4-{5-[3-chloro-5-(trifluoromethyl)phenyl]-5-(trifluoromethyl)-4,5-dihydro-1,2-oxazol-3-yl}-N-{2-oxo-2-[(2,2,2-trifluoroethyl)amino]ethyl}-1-naphthamide (known from WO2009/002809), methyl 2-[2-({[3-bromo-1-(3-chloropyridin-2-yl)-1H-pyrazol-5-yl]carbonyl}amino)-5-chloro-3-methylbenzoyl]-2-methylhydrazinecarboxylate (known from WO2005/085216), methyl 2-[2-({[3-bromo-1-(3-chloropyridin-2-yl)-1H-pyrazol-5-yl]carbonyl}amino)-5-cyano-3-methylbenzoyl]-2-ethylhydrazinecarboxylate (known from WO2005/085216), methyl 2-[2-({[3-bromo-1-(3-chloropyridin-2-yl)-1H-pyrazol-5-yl]carbonyl}amino)-5-cyano-3-methylbenzoyl]-2-methylhydrazinecarboxylate (known from WO2005/085216), methyl 2-[3,5-dibromo-2-({[3-bromo-1-(3-chloropyridin-2-yl)-1H-pyrazol-5-yl]carbonyl}amino)benzoyl]-2-ethylhydrazinecarboxylate (known from WO2005/085216), 1-(3-chloropyridin-2-yl)-N-[4-cyano-2-methyl-6-(methylcarbamoyl)phenyl]-3-{[5-(trifluoromethyl)-2H-tetrazol-2-yl]methyl}-1H-pyrazole-5-carboxamide (known from WO2010/069502), N-[2-(5-amino-1,3,4-thiadiazol-2-yl)-4-chloro-6-methylphenyl]-3-bromo-1-(3-chloropyridin-2-yl)-1H-pyrazole-5-carboxamide (known from CN102057925), 3-chloro-N-(2-cyanopropan-2-yl)-N-[4-(1,1,1,2,3,3,3-heptafluoropropan-2-yl)-2-methylphenyl]phthalamide (known from WO2012/034472), 8-chloro-N-[(2-chloro-5-methoxyphenyl)sulphonyl]-6-(trifluoromethyl)imidazo[1,2-a]pyridine-2-carboxamide (known from WO2010/129500), 4-[5-(3,5-dichlorophenyl)-5-(trifluoromethyl)-4,5-dihydro-1,2-oxazol-3-yl]-2-methyl-N-(1-oxidothietan-3-yl)benzamide (known from WO2009/080250), N-[(2E)-1-[(6-chloropyridin-3-yl)methyl]pyridin-2(1H)-ylidene]-2,2,2-trifluoracetamide (known from WO2012/029672), 1-[(2-chloro-1,3-thiazol-5-yl)methyl]-4-oxo-3-phenyl-4H-pyrido[1,2-a]pyrimidin-1-ium-2-olate (known from WO2009/099929), 1-[(6-chloropyridin-3-yl)methyl]-4-oxo-3-phenyl-4H-pyrido[1,2-a]pyrimidin-1-ium-2-olate (known from WO2009/099929), (5S,8R)-1-[(6-chloropyridin-3-yl)methyl]-9-nitro-2,3,5,6,7,8-hexahydro-1H-5,8-epoxyimidazo [1,2-a]azepine (known from WO2010/069266), (2E)-1-[(6-chloropyridin-3-yl)methyl]-N′-nitro-2-pentylidenehydrazinecarboximidamide (known from WO2010/060231), 4-(3-{2,6-dichloro-4-[(3,3-dichloroprop-2-en-1-yl)oxy]phenoxy}propoxy)-2-methoxy-6-(trifluoromethyl)pyrimidine (known from CN101337940), N-[2-(tert-butylcarbamoyl)-4-chloro-6-methylphenyl]-1-(3-chloropyridin-2-yl)-3-(fluoromethoxy)-1H-pyrazole-5-carboxamide (known from WO2008/134969).

### Fungicides

The active compounds specified herein by their common name are known and described, for example, in “Pesticide Manual” or on the Internet (for example: http://www.alanwood.net/pesticides).

All fungicidal mixing partners of classes (1) to (15) mentioned may, their functional groups permitting, optionally form salts with suitable bases or acids. All mixing partners of classes (1) to (15) mentioned may, if applicable, include tautomeric forms.

(1) Ergosterol biosynthesis inhibitors, for example (1.1) aldimorph, (1.2) azaconazole, (1.3) bitertanol, (1.4) bromuconazole, (1.5) cyproconazole, (1.6) diclobutrazole, (1.7) difenoconazole, (1.8) diniconazole, (1.9) diniconazole-M, (1.10) dodemorph, (1.11) dodemorph acetate, (1.12) epoxiconazole, (1.13) etaconazole, (1.14) fenarimol, (1.15) fenbuconazole, (1.16) fenhexamid, (1.17) fenpropidin, (1.18) fenpropimorph, (1.19) fluquinconazole, (1.20) flurprimidol, (1.21) flusilazole, (1.22) flutriafole, (1.23) furconazole, (1.24) furconazole-cis, (1.25) hexaconazole, (1.26) imazalil, (1.27) imazalil sulphate, (1.28) imibenconazole, (1.29) ipconazole, (1.30) metconazole, (1.31) myclobutanil, (1.32) naftifin, (1.33) nuarimol, (1.34) oxpoconazole, (1.35) paclobutrazole, (1.36) pefurazoate, (1.37) penconazole, (1.38) piperalin, (1.39) prochloraz, (1.40) propiconazole, (1.41) prothioconazole, (1.42) pyributicarb, (1.43) pyrifenox, (1.44) quinconazole, (1.45) simeconazole, (1.46) spiroxamine, (1.47) tebuconazole, (1.48) terbinafin, (1.49) tetraconazole, (1.50) triadimefon, (1.51) triadimenol, (1.52) tridemorph, (1.53) triflumizole, (1.54) triforine, (1.55) triticonazole, (1.56) uniconazole, (1.57) uniconazole-P, (1.58) viniconazole, (1.59) voriconazole, (1.60) 1-(4-chlorophenyl)-2-(1H-1,2,4-triazol-1-yl)cycloheptanol, (1.61) methyl 1-(2,2-dimethyl-2,3-dihydro-1H-inden-1-yl)-1H-imidazole-5-carboxylate, (1.62) N′-{5-(difluoromethyl)-2-methyl-4-[3-(trimethylsilyl)propoxy]phenyl}-N-ethyl-N-methylimidoformamide, (1.63) N-ethyl-N-methyl-N′-{2-methyl-5-(trifluoromethyl)-4-[3-(trimethylsilyl)propoxy]phenyl}imidoformamide and (1.64) 0-[1-(4-methoxyphenoxy)-3,3-dimethylbutan-2-yl]-1H-imidazole-1-carbothioate, (1.65) pyrisoxazole.

(2) Respiration inhibitors (respiratory chain inhibitors), for example (2.1) bixafen, (2.2) boscalid, (2.3) carboxin, (2.4) diflumetorim, (2.5) fenfuram, (2.6) fluopyram, (2.7) flutolanil, (2.8) fluxapyroxad, (2.9) furametpyr, (2.10) furmecyclox, (2.11) isopyrazam mixture of the syn-epimeric racemate 1RS,4SR,9RS and the anti-empimeric racemate 1RS,4SR,9SR, (2.12) isopyrazam (anti-epimeric racemate), (2.13) isopyrazam (anti-epimeric enantiomer 1R,4S,9S), (2.14) isopyrazam (anti-epimeric enantiomer 1S,4R,9R), (2.15) isopyrazam (syn-epimeric racemate 1RS,4SR,9RS), (2.16) isopyrazam (syn-epimeric enantiomer 1R,4S,9R), (2.17) isopyrazam (syn-epimeric enantiomer 1S,4R,9S), (2.18) mepronil, (2.19) oxycarboxin, (2.20) penflufen, (2.21) penthiopyrad, (2.22) sedaxane, (2.23) thifluzamide, (2.24) 1-methyl-N-[2-(1,1,2,2-tetrafluoroethoxy)phenyl]-3-(trifluoromethyl)-1H-pyrazole-4-carboxamide, (2.25) 3-(difluoromethyl)-1-methyl-N-[2-(1,1,2,2-tetrafluoroethoxy)phenyl]-1H-pyrazole-4-carboxamide, (2.26) 3-(difluoromethyl)-N-[4-fluoro-2-(1,1,2,3,3,3-hexafluoropropoxy)phenyl]-1-methyl-1H-pyrazole-4-carboxamide, (2.27) N-[1-(2,4-dichlorophenyl)-1-methoxypropan-2-yl]-3-(difluoromethyl)-1-methyl-1H-pyrazole-4-carboxamide, (2.28) 5,8-difluoro-N-[2-(2-fluoro-4-{[4-(trifluoromethyl)pyridin-2-yl]oxy}phenyl)ethyl]quinazoline-4-amine, (2.29) benzovindiflupyr, (2.30) N-[(1S,4R)-9-(dichloromethylene)-1,2,3,4-tetrahydro-1,4-methanonaphthalen-5-yl]-3-(difluoromethyl)-1-methyl-1H-pyrazole-4-carboxamide and (2.31) N-[(1R,4S)-9-(dichloromethylene)-1,2,3,4-tetrahydro-1,4-methanonaphthalen-5-yl]-3-(difluoromethyl)-1-methyl-1H-pyrazole-4-carboxamide, (2.32) 3-(difluoromethyl)-1-methyl-N-(1,1,3-trimethyl-2,3-dihydro-1H-inden-4-yl)-1H-pyrazole-4-carboxamide, (2.33) 1,3,5-trimethyl-N-(1,1,3-trimethyl-2,3-dihydro-1H-inden-4-yl)-1H-pyrazole-4-carboxamide, (2.34) 1-methyl-3-(trifluoromethyl)-N-(1,1,3-trimethyl-2,3-dihydro-1H-inden-4-yl)-1H-pyrazole-4-carboxamide, (2.35) 1-methyl-3-(trifluoromethyl)-N-[(3R)-1,1,3-trimethyl-2,3-dihydro-1H-inden-4-yl]-1H-pyrazole-4-carboxamide, (2.36) 1-methyl-3-(trifluoromethyl)-N-[(3S)-1,1,3-trimethyl-2,3-dihydro-1H-inden-4-yl]-1H-pyrazole-4-carboxamide, (2.37) 3-(difluoromethyl)-1-methyl-N-[(3S)-1,1,3-trimethyl-2,3-dihydro-1H-inden-4-yl]-1H-pyrazole-4-carboxamide, (2.38) 3-(difluoromethyl)-1-methyl-N-[(3R)-1,1,3-trimethyl-2,3-dihydro-1H-inden-4-yl]-1H-pyrazole-4-carboxamide, (2.39) 1,3,5-trimethyl-N-[(3R)-1,1,3-trimethyl-2,3-dihydro-1H-inden-4-yl]-1H-pyrazole-4-carboxamide, (2.40) 1,3,5-trimethyl-N-[(3S)-1,1,3-trimethyl-2,3-dihydro-1H-inden-4-yl]-1H-pyrazole-4-carboxamide, (2.41) benodanil, (2.42) 2-chloro-N-(1,1,3-trimethyl-2,3-dihydro-1H-inden-4-yl)pyridine-3-carboxamide, (2.43) isofetamid

(3) Respiration inhibitors (respiratory chain inhibitors) that act on complex III of the respiratory chain, for example (3.1) ametoctradin, (3.2) amisulbrom, (3.3) azoxystrobin, (3.4) cyazofamid, (3.5) coumethoxystrobin, (3.6) coumoxystrobin, (3.7) dimoxystrobin, (3.8) enestroburin, (3.9) famoxadone, (3.10) fenamidone, (3.11) flufenoxystrobin, (3.12) fluoxastrobin, (3.13) kresoxim-methyl, (3.14) metominostrobin, (3.15) orysastrobin, (3.16) picoxystrobin, (3.17) pyraclostrobin, (3.18) pyrametostrobin, (3.19) pyraoxystrobin, (3.20) pyribencarb, (3.21) triclopyricarb, (3.22) trifloxystrobin, (3.23) (2E)-2-(2-{[6-(3-chloro-2-methylphenoxy)-5-fluoropyrimidin-4-yl]oxy}phenyl)-2-(methoxyimino)-N-methylethanamide, (3.24) (2E)-2-(methoxyimino)-N-methyl-2-(2-{[({(1E)-1-[3-(trifluoromethyl)phenyl]ethylidene}amino)oxy]methyl}phenyl)ethanamide, (3.25) (2E)-2-(methoxyimino)-N-methyl-2-{2-[(E)-({1-[3-(trifluoromethyl)phenyl]ethoxy}imino)methyl]phenyl}ethanamide, (3.26) (2E)-2-{2-[({[(1E)-1-(3-{[(E)-1-fluoro-2-phenylethenyl]oxy}phenyl)ethylidene]amino}oxy)methyl]phenyl}-2-(methoxyimino)-N-methylethanamide, (3.27) (2E)-2-{2-[({[(2E,3E)-4-(2,6-dichlorophenyl)but-3-en-2-ylidene]amino}oxy)methyl]phenyl}-2-(methoxyimino)-N-methylethanamide, (3.28) 2-chloro-N-(1,1,3-trimethyl-2,3-dihydro-1H-inden-4-yl)pyridine-3-carboxamide, (3.29) 5-methoxy-2-methyl-4-(2-{[({(1E)-1-[3-(trifluoromethyl)phenyl]ethylidene}amino)oxy]methyl}phenyl)-2,4-dihydro-3H-1,2,4-triazol-3-one, (3.30) methyl (2E)-2-{2-[({cyclopropyl[(4-methoxyphenyl)imino]methyl}sulphanyl)methyl]phenyl}-3-methoxyprop-2-enoate, (3.31) N-(3-ethyl-3,5,5-trimethylcyclohexyl)-3-(formylamino)-2-hydroxybenzamide, (3.32) 2-{2-[(2,5-dimethylphenoxy)methyl]phenyl}-2-methoxy-N-methylacetamide (4) inhibitors of mitosis and cell division, for example (4.1) benomyl, (4.2) carbendazim, (4.3) chlorfenazole, (4.4) diethofencarb, (4.5) ethaboxam, (4.6) fluopicolid, (4.7) fuberidazole, (4.8) pencycuron, (4.9) thiabendazole, (4.10) thiophanate-methyl, (4.11) thiophanate, (4.12) zoxamide, (4.13) 5-chloro-7-(4-methylpiperidin-1-yl)-6-(2,4,6-trifluorophenyl)[1,2,4]triazolo[1,5-a]pyrimidine and (4.14) 3-chloro-5-(6-chloropyridin-3-yl)-6-methyl-4-(2,4,6-trifluorophenyl)pyridazine.

(5) Compounds having multisite activity, for example (5.1) Bordeaux mixture, (5.2) captafol, (5.3) captan, (5.4) chlorothalonil, (5.5) copper preparations such as copper hydroxide, (5.6) copper naphthenate, (5.7) copper oxide, (5.8) copper oxychloride, (5.9) copper sulphate, (5.10) dichlofluanid, (5.11) dithianon, (5.12) dodine, (5.13) dodine free base, (5.14) ferbam, (5.15) fluorfolpet, (5.16) folpet, (5.17) guazatine, (5.18) guazatine acetate, (5.19) iminoctadine, (5.20) iminoctadine albesilate, (5.21) iminoctadine triacetate, (5.22) mancopper, (5.23) mancozeb, (5.24) maneb, (5.25) metiram, (5.26) zinc metiram, (5.27) copper-oxine, (5.28) propamidine, (5.29) propineb, (5.30) sulphur and sulphur preparations, for example calcium polysulphide, (5.31) thiram, (5.32) tolylfluanid, (5.33) zineb, (5.34) ziram and (5.35) anilazine.

(6) Resistance inducers, for example (6.1) acibenzolar-S-methyl, (6.2) isotianil, (6.3) probenazole, (6.4) tiadinil and (6.5) laminarin.

(7) Amino acid and protein biosynthesis inhibitors, for example (7.1), (7.2) blasticidin-S, (7.3) cyprodinil, (7.4) kasugamycin, (7.5) kasugamycin hydrochloride hydrate, (7.6) mepanipyrim, (7.7) pyrimethanil, (7.8) 3-(5-fluoro-3,3,4,4-tetramethyl-3,4-dihydroisoquinolin-1-yl)quinoline and (7.9) oxytetracycline and (7.10) streptomycin.

(8) ATP production inhibitors such as, for example, (8.1) fentin acetate, (8.2) fentin chloride, (8.3) fentin hydroxide and (8.4) silthiofam.

(9) Cell wall synthesis inhibitors, for example (9.1) benthiavalicarb, (9.2) dimethomorph, (9.3) flumorph, (9.4) iprovalicarb, (9.5) mandipropamid, (9.6) polyoxins, (9.7) polyoxorim, (9.8) validamycin A, (9.9) valifenalate and (9.10) polyoxin B.

(10) Lipid and membrane synthesis inhibitors, for example (10.1) biphenyl, (10.2) chlorneb, (10.3) dicloran, (10.4) edifenphos, (10.5) etridiazole, (10.6) iodocarb, (10.7) iprobenfos, (10.8) isoprothiolane, (10.9) propamocarb, (10.10) propamocarb hydrochloride, (10.11) prothiocarb, (10.12) pyrazophos, (10.13) quintozene, (10.14) tecnazene and (10.15) tolclofos-methyl.

(11) Melanin biosynthesis inhibitors, for example (11.1) carpropamid, (11.2) diclocymet, (11.3) fenoxanil, (11.4) fthalide, (11.5) pyroquilon, (11.6) tricyclazole and (11.7) 2,2,2-trifluoroethyl {3-methyl-1-[(4-methylbenzoyl)amino]butan-2-yl}carbamate.

(12) Nucleic acid synthesis inhibitors, for example (12.1) benalaxyl, (12.2) benalaxyl-M (kiralaxyl), (12.3) bupirimate, (12.4) clozylacon, (12.5) dimethirimol, (12.6) ethirimol, (12.7) furalaxyl, (12.8) hymexazole, (12.9) metalaxyl, (12.10) metalaxyl-M (mefenoxam), (12.11) ofurace, (12.12) oxadixyl, (12.13) oxolinic acid and (12.14) octhilinone.

(13) Signal transduction inhibitors, for example (13.1) chlozolinate, (13.2) fenpiclonil, (13.3) fludioxonil, (13.4) iprodione, (13.5) procymidone, (13.6) quinoxyfen, (13.7) vinclozolin and (13.8) proquinazid.

(14) Decouplers, for example (14.1) binapacryl, (14.2) dinocap, (14.3) ferimzone, (14.4) fluazinam and (14.5) meptyldinocap.

(15) Further compounds, for example (15.1) benthiazole, (15.2) bethoxazine, (15.3) capsimycin, (15.4) carvone, (15.5) quinomethionate, (15.6) pyriofenone (chlazafenone), (15.7) cufraneb, (15.8) cyflufenamid, (15.9) cymoxanil, (15.10) cyprosulphamide, (15.11) dazomet, (15.12) debacarb, (15.13) dichlorophen, (15.14) diclomezine, (15.15) difenzoquat, (15.16) difenzoquat methylsulphate, (15.17) diphenylamine, (15.18) EcoMate, (15.19) fenpyrazamine, (15.20) flumetover, (15.21) fluorimid, (15.22) flusulphamide, (15.23) flutianil, (15.24) fosetyl-aluminium, (15.25) fosetyl-calcium, (15.26) fosetyl-sodium, (15.27) hexachlorobenzene, (15.28) irumamycin, (15.29) methasulphocarb, (15.30) methyl isothiocyanate, (15.31) metrafenone, (15.32) mildiomycin, (15.33) natamycin, (15.34) nickel dimethyldithiocarbamate, (15.35) nitrothal-isopropyl, (15.36) octhilinone, (15.37) oxamocarb, (15.38) oxyfenthiin, (15.39) pentachlorophenol and its salts, (15.40) phenothrin, (15.41) phosphoric acid and its salts, (15.42) propamocarb-fosetylate, (15.43) propanosine-sodium, (15.44) pyrimorph, (15.45) (2E)-3-(4-tert-butylphenyl)-3-(2-chloropyridin-4-yl)-1-(morpholin-4-yl)prop-2-en-1-one, (15.46) (2Z)-3-(4-tert-butylphenyl)-3-(2-chloropyridin-4-yl)-1-(morpholin-4-yl)prop-2-en-1-one, (15.47) pyrrolnitrin, (15.48) tebufloquin, (15.49) tecloftalam, (15.50) tolnifanide, (15.51) triazoxide, (15.52) trichlamide, (15.53) zarilamid, (15.54) (3S,6S,7R,8R)-8-benzyl-3-[({3-[(isobutyryloxy)methoxy]-4-methoxypyridin-2-yl}carbonyl)amino]-6-methyl-4,9-dioxo-1,5-dioxonan-7-yl 2-methylpropanoate, (15.55) 1-(4-{4-[(5R)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (15.56) 1-(4-{4-[(5S)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (15.57) 1-(4-{4-[5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (15.58) 1-(4-methoxyphenoxy)-3,3-dimethylbutan-2-yl 1H-imidazole-1-carboxylate, (15.59) 2,3,5,6-tetrachloro-4-(methylsulphonyl)pyridine, (15.60) 2,3-dibutyl-6-chlorothieno[2,3-d]pyrimidin-4(3H)-one, (15.61) 2,6-dimethyl-1H,5H-[1,4]dithiino[2,3-c:5,6-c′]dipyrrole-1,3,5,7(2H,6H)-tetrone, (15.62) 2-[5-methyl-3-(trifluoromethyl)-1H-pyrazol-1-yl]-1-(4-{4-[(5R)-5-phenyl-4,5-dihydro-1,2-oxazol-3-yl]-1,3-thiazol-2-yl}piperidin-1-yl)ethanone, (15.63) 2-[5-methyl-3-(trifluoromethyl)-1H-pyrazol-1-yl]-1-(4-{4-[(5S)-5-phenyl-4,5-dihydro-1,2-oxazol-3-yl]-1,3-triazol-2-yl}piperidin-1-yl)ethanone, (15.64) 2-[5-methyl-3-(trifluoromethyl)-1H-pyrazol-1-yl]-1-{4-[4-(5-phenyl-4,5-dihydro-1,2-oxazol-3-yl)-1,3-thiazol-2-yl]piperidin-1-yl}ethanone, (15.65) 2-butoxy-6-iodo-3-propyl-4H-chromen-4-one, (15.66) 2-chloro-5-[2-chloro-1-(2,6-difluoro-4-methoxyphenyl)-4-methyl-1H-imidazol-5-yl]pyridine, (15.67) 2-phenylphenol and salts, (15.68) 3-(4,4,5-trifluoro-3,3-dimethyl-3,4-dihydroisoquinolin-1-yl)quinoline, (15.69) 3,4,5-trichloropyridine-2,6-dicarbonitrile, (15.70) 3-chloro-5-(4-chlorophenyl)-4-(2,6-difluorophenyl)-6-methylpyridazine, (15.71) 4-(4-chlorophenyl)-5-(2,6-difluorophenyl)-3,6-dimethylpyridazine, (15.72) 5-amino-1,3,4-thiadiazole-2-thiol, (15.73) 5-chloro-N′-phenyl-N′-(prop-2-yn-1-yl)thiophene-2-sulphonohydrazide, (15.74) 5-fluoro-2-[(4-fluorobenzyl)oxy]pyrimidine-4-amine, (15.75) 5-fluoro-2-[(4-methylbenzyl)oxy]pyrimidine-4-amine, (15.76) 5-methyl-6-octyl[1,2,4]triazolo[1,5-a]pyrimidine-7-amine, (15.77) ethyl (2Z)-3-amino-2-cyano-3-phenylacrylate, (15.78) N′-(4-{[3-(4-chlorobenzyl)-1,2,4-thiadiazol-5-yl]oxy}-2,5-dimethylphenyl)-N-ethyl-N-methylimidoformamide, (15.79) N-(4-chlorobenzyl)-3-[3-methoxy-4-(prop-2-yn-1-yloxy)phenyl]propanamide, (15.80) N-[(4-chlorophenyl)(cyano)methyl]-3-[3-methoxy-4-(prop-2-yn-1-yloxy)phenyl]propanamide, (15.81) N-[(5-bromo-3-chloropyridin-2-yl)methyl]-2,4-dichloronicotinamide, (15.82) N-[1-(5-bromo-3-chloropyridin-2-yl)ethyl]-2,4-dichloronicotinamide, (15.83) N-[1-(5-bromo-3-chloropyridin-2-yl)ethyl]-2-fluoro-4-iodonicotinamide, (15.84) N-{(E)-[(cyclopropylmethoxy)imino][6-(difluoromethoxy)-2,3-difluorophenyl]methyl}-2-phenylacetamide, (15.85) N-{(Z)-[(cyclopropylmethoxy)imino][6-(difluoromethoxy)-2,3-difluorophenyl]methyl}-2-phenylacetamide, (15.86) N′-{4-[(3-tert-butyl-4-cyano-1,2-thiazol-5-yl)oxy]-2-chloro-5-methylphenyl}-N-ethyl-N-methylimidoformamide, (15.87) N-methyl-2-(1-{[5-methyl-3-(trifluoromethyl)-1H-pyrazol-1-yl]acetyl}piperidin-4-yl)-N-(1,2,3,4-tetrahydronaphthalen-1-yl)-1,3-thiazole-4-carboxamide, (15.88) N-methyl-2-(1-{[5-methyl-3-(trifluoromethyl)-1H-pyrazol-1-yl]acetyl}piperidin-4-yl)-N-[(1R)-1,2,3,4-tetrahydronaphthalen-1-yl]-1,3-thiazole-4-carboxamide, (15.89) N-methyl-2-(1-{[5-methyl-3-(trifluoromethyl)-1H-pyrazol-1-yl]acetyl}piperidin-4-yl)-N-[(1 S)-1,2,3,4-tetrahydronaphthalen-1-yl]-1,3-thiazole-4-carboxamide, (15.90) pentyl {6-[({[(1-methyl-1H-tetrazol-5-yl)(phenyl)methylene]amino}oxy)methyl]pyridin-2-yl}carbamate, (15.91) phenazine-1-carboxylic acid, (15.92) quinolin-8-ol, (15.93) quinolin-8-ol sulphate (2:1), (15.94) tert-butyl {6-[({[(1-methyl-1H-tetrazol-5-yl)(phenyl)methylene]amino}oxy)methyl]pyridin-2-yl}carbamate, (15.95) 1-methyl-3-(trifluoromethyl)-N-[2′-(trifluoromethyl)biphenyl-2-yl]-1H-pyrazole-4-carboxamide, (15.96) N-(4′-chlorobiphenyl-2-yl)-3-(difluoromethyl)-1-methyl-1H-pyrazole-4-carboxamide, (15.97) N-(2′,4′-dichlorobiphenyl-2-yl)-3-(difluoromethyl)-1-methyl-1H-pyrazole-4-carboxamide, (15.98) 3-(difluoromethyl)-1-methyl-N-[4′-(trifluoromethyl)biphenyl-2-yl]-1H-pyrazole-4-carboxamide, (15.99) N-(2′,5′-difluorobiphenyl-2-yl)-1-methyl-3-(trifluoromethyl)-1H-pyrazole-4-carboxamide, (15.100) 3-(difluoromethyl)-1-methyl-N-[4′-(prop-1-yn-1-yl)biphenyl-2-yl]-1H-pyrazole-4-carboxamide, (15.101) 5-fluoro-1,3-dimethyl-N-[4′-(prop-1-yn-1-yl)biphenyl-2-yl]-1H-pyrazole-4-carboxamide, (15.102) 2-chloro-N-[4′-(prop-1-yn-1-yl)biphenyl-2-yl]nicotinamide, (15.103) 3-(difluoromethyl)-N-[4′-(3,3-dimethylbut-1-yn-1-yl)biphenyl-2-yl]-1-methyl-1H-pyrazole-4-carboxamide, (15.104) N-[4′-(3,3-dimethylbut-1-yn-1-yl)biphenyl-2-yl]-5-fluoro-1,3-dimethyl-1H-pyrazole-4-carboxamide, (15.105) 3-(difluoromethyl)-N-(4′-ethynylbiphenyl-2-yl)-1-methyl-1H-pyrazole-4-carboxamide, (15.106) N-(4′-ethynylbiphenyl-2-yl)-5-fluoro-1,3-dimethyl-1H-pyrazole-4-carboxamide, (15.107) 2-chloro-N-(4′-ethynylbiphenyl-2-yl)nicotinamide, (15.108) 2-chloro-N-[4′-(3,3-dimethylbut-1-yn-1-yl)biphenyl-2-yl]nicotinamide, (15.109) 4-(difluoromethyl)-2-methyl-N-[4′-(trifluoromethyl)biphenyl-2-yl]-1,3-thiazole-5-carboxamide, (15.110) 5-fluoro-N-[4′-(3-hydroxy-3-methylbut-1-yn-1-yl)biphenyl-2-yl]-1,3-dimethyl-1H-pyrazole-4-carboxamide, (15.111) 2-chloro-N-[4′-(3-hydroxy-3-methylbut-1-yn-1-yl)biphenyl-2-yl]nicotinamide, (15.112) 3-(difluoromethyl)-N-[4′-(3-methoxy-3-methylbut-1-yn-1-yl)biphenyl-2-yl]-1-methyl-1H-pyrazole-4-carboxamide, (15.113) 5-fluoro-N-[4′-(3-methoxy-3-methylbut-1-yn-1-yl)biphenyl-2-yl]-1,3-dimethyl-1H-pyrazole-4-carboxamide, (15.114) 2-chloro-N-[4′-(3-methoxy-3-methylbut-1-yn-1-yl)biphenyl-2-yl]nicotinamide, (15.115) (5-bromo-2-methoxy-4-methylpyridin-3-yl)(2,3,4-trimethoxy-6-methylphenyl)methanone, (15.116) N-[2-(4-{[3-(4-chlorophenyl)prop-2-yn-1-yl]oxy}-3-methoxyphenyl)ethyl]-N2-(methylsulphonyl)valinamide, (15.117) 4-oxo-4-[(2-phenylethyl)amino]butanoic acid, (15.118) but-3-yn-1-yl {6-[({[(Z)-(1-methyl-1H-tetrazol-5-yl)(phenyl)methylene]amino}oxy)methyl]pyridin-2-yl}carbamate, (15.119) 4-amino-5-fluoropyrimidin-2-ol (tautomeric form: 4-amino-5-fluoropyrimidin-2(1H)-one), (15.120) propyl 3,4,5-trihydroxybenzoate, (15.121) 1,3-dimethyl-N-(1,1,3-trimethyl-2,3-dihydro-1H-inden-4-yl)-1H-pyrazole-4-carboxamide, (15.122) 1,3-dimethyl-N-[(3R)-1,1,3-trimethyl-2,3-dihydro-1H-inden-4-yl]-1H-pyrazole-4-carboxamide, (15.123) 1,3-dimethyl-N-[(3 S)-1,1,3-trimethyl-2,3-dihydro-1H-inden-4-yl]-1H-pyrazole-4-carboxamide, (15.124) [3-(4-chloro-2-fluorophenyl)-5-(2,4-difluorophenyl)-1,2-oxazol-4-yl](pyridin-3-yl)methanol, (15.125) (S)-[3-(4-chloro-2-fluorophenyl)-5-(2,4-difluorophenyl)-1,2-oxazol-4-yl](pyridin-3-yl)methanol, (15.126) (R)-[3-(4-chloro-2-fluorophenyl)-5-(2,4-difluorophenyl)-1,2-oxazol-4-yl](pyridin-3-yl)methanol, (15.127) 2-{[3-(2-chlorophenyl)-2-(2,4-difluorophenyl)oxiran-2-yl]methyl}-2,4-dihydro-3H-1,2,4-triazole-3-thione, (15.128) 1-{[3-(2-chlorophenyl)-2-(2,4-difluorophenyl)oxiran-2-yl]methyl}-1H-1,2,4-triazol-5-yl thiocyanate, (15.129) 5-(allylsulphanyl)-1-{[3-(2-chlorophenyl)-2-(2,4-difluorophenyl)oxiran-2-yl]methyl}-1H-1,2,4-triazole, (15.130) 2-[1-(2,4-dichlorophenyl)-5-hydroxy-2,6,6-trimethylheptan-4-yl]-2,4-dihydro-3H-1,2,4-triazole-3-thione, (15.131) 2-{[rel(2R,3S)-3-(2-chlorophenyl)-2-(2,4-difluorophenyl)oxiran-2-yl]methyl}-2,4-dihydro-3H-1,2,4-triazole-3-thione, (15.132) 2-{[rel(2R,3R)-3-(2-chlorophenyl)-2-(2,4-difluorophenyl)oxiran-2-yl]methyl}-2,4-dihydro-3H-1,2,4-triazole-3-thione, (15.133) 1-{[rel(2R,3 S)-3-(2-chlorophenyl)-2-(2,4-difluorophenyl)oxiran-2-yl]methyl}-1H-1,2,4-triazol-5-yl thiocyanate, (15.134) 1-{[rel(2R,3R)-3-(2-chlorophenyl)-2-(2,4-difluorophenyl)oxiran-2-yl]methyl}-1H-1,2,4-triazol-5-yl thiocyanate, (15.135) 5-(allylsulphanyl)-1-{[rel(2R,3S)-3-(2-chlorophenyl)-2-(2,4-difluorophenyl)oxiran-2-yl]methyl}-1H-1,2,4-triazole, (15.136) 5-(allylsulphanyl)-1-{[rel(2R,3R)-3-(2-chlorophenyl)-2-(2,4-difluorophenyl)oxiran-2-yl]methyl}-1H-1,2,4-triazole, (15.137) 2-[(2S,4S,5S)-1-(2,4-dichlorophenyl)-5-hydroxy-2,6,6-trimethylheptan-4-yl]-2,4-dihydro-3H-1,2,4-triazole-3-thione, (15.138) 2-[(2R,4S,5 S)-1-(2,4-dichlorophenyl)-5-hydroxy-2,6,6-trimethylheptan-4-yl]-2,4-dihydro-3H-1,2,4-triazole-3-thione, (15.139) 2-[(2R,4R,5R)-1-(2,4-dichlorophenyl)-5-hydroxy-2,6,6-trimethylheptan-4-yl]-2,4-dihydro-3H-1,2,4-triazole-3-thione, (15.140) 2-[(2S,4R,5R)-1-(2,4-dichlorophenyl)-5-hydroxy-2,6,6-trimethylheptan-4-yl]-2,4-dihydro-3H-1,2,4-triazole-3-thione, (15.141) 2-[(2S,4S,5R)-1-(2,4-dichlorophenyl)-5-hydroxy-2,6,6-trimethylheptan-4-yl]-2,4-dihydro-3H-1,2,4-triazole-3-thione, (15.142) 2-[(2R,4S,5R)-1-(2,4-dichlorophenyl)-5-hydroxy-2,6,6-trimethylheptan-4-yl]-2,4-dihydro-3H-1,2,4-triazole-3-thione, (15.143) 2-[(2R,4R,5 S)-1-(2,4-dichlorophenyl)-5-hydroxy-2,6,6-trimethylheptan-4-yl]-2,4-dihydro-3H-1,2,4-triazole-3-thione, (15.144) 2-[(2S,4R,5S)-1-(2,4-dichlorophenyl)-5-hydroxy-2,6,6-trimethylheptan-4-yl]-2,4-dihydro-3H-1,2,4-triazole-3-thione, (15.145) 2-fluoro-6-(trifluoromethyl)-N-(1,1,3-trimethyl-2,3-dihydro-1H-inden-4-yl)benzamide, (15.146) 2-(6-benzylpyridin-2-yl)quinazoline, (15.147) 2-[6-(3-fluoro-4-methoxyphenyl)-5-methylpyridin-2-yl]quinazoline, (15.148) 3-(4,4-difluoro-3,3-dimethyl-3,4-dihydroisoquinolin-1-yl)quinoline, (15.149) abscisic acid, (15.150) 3-(difluoromethyl)-N-methoxy-1-methyl-N-[1-(2,4,6-trichlorophenyl)propan-2-yl]-1H-pyrazole-4-carboxamide, (15.151) N′-[5-bromo-6-(2,3-dihydro-1H-inden-2-yloxy)-2-methylpyridin-3-yl]-N-ethyl-N-methylimidoformamide, (15.152) N′-{5-bromo-6-[1-(3,5-difluorophenyl)ethoxy]-2-methylpyridin-3-yl}-N-ethyl-N-methylimidoformamide, (15.153) N′-{5-bromo-6-[(1R)-1-(3,5-difluorophenyl)ethoxy]-2-methylpyridin-3-yl}-N-ethyl-N-methylimidoformamide, (15.154) N′-{5-bromo-6-[(1S)-1-(3,5-difluorophenyl)ethoxy]-2-methylpyridin-3-yl}-N-ethyl-N-methylimidoformamide, (15.155) N′-{5-bromo-6-[(cis-4-isopropylcyclohexyl)oxy]-2-methylpyridin-3-yl}-N-ethyl-N-methylimidoformamide, (15.156) N′-{5-bromo-6-[(trans-4-isopropylcyclohexyl)oxy]-2-methylpyridin-3-yl}-N-ethyl-N-methylimidoformamide, (15.157) N-cyclopropyl-3-(difluoromethyl)-5-fluoro-N-(2-isopropylbenzyl)-1-methyl-1H-pyrazole-4-carboxamide, (15.158) N-cyclopropyl-N-(2-cyclopropylbenzyl)-3-(difluoromethyl)-5-fluoro-1-methyl-1H-pyrazole-4-carboxamide, (15.159) N-(2-tert-butylbenzyl)-N-cyclopropyl-3-(difluoromethyl)-5-fluoro-1-methyl-1H-pyrazole-4-carboxamide, (15.160) N-(5-chloro-2-ethylbenzyl)-N-cyclopropyl-3-(difluoromethyl)-5-fluoro-1-methyl-1H-pyrazole-4-carboxamide, (15.161) N-(5-chloro-2-isopropylbenzyl)-N-cyclopropyl-3-(difluoromethyl)-5-fluoro-1-methyl-1H-pyrazole-4-carboxamide, (15.162) N-cyclopropyl-3-(difluoromethyl)-N-(2-ethyl-5-fluorobenzyl)-5-fluoro-1-methyl-1H-pyrazole-4-carboxamide, (15.163) N-cyclopropyl-3-(difluoromethyl)-5-fluoro-N-(5-fluoro-2-isopropylbenzyl)-1-methyl-1H-pyrazole-4-carboxamide, (15.164) N-cyclopropyl-N-(2-cyclopropyl-5-fluorobenzyl)-3-(difluoromethyl)-5-fluoro-1-methyl-1H-pyrazole-4-carboxamide, (15.165) N-(2-cyclopentyl-5-fluorobenzyl)-N-cyclopropyl-3-(difluoromethyl)-5-fluoro-1-methyl-1H-pyrazole-4-carboxamide, (15.166) N-cyclopropyl-3-(difluoromethyl)-5-fluoro-N-(2-fluoro-6-isopropylbenzyl)-1-methyl-1H-pyrazole-4-carboxamide, (15.167) N-cyclopropyl-3-(difluoromethyl)-N-(2-ethyl-5-methylbenzyl)-5-fluoro-1-methyl-1H-pyrazole-4-carboxamide, (15.168) N-cyclopropyl-3-(difluoromethyl)-5-fluoro-N-(2-isopropyl-5-methylbenzyl)-1-methyl-1H-pyrazole-4-carboxamide, (15.169) N-cyclopropyl-N-(2-cyclopropyl-5-methylbenzyl)-3-(difluoromethyl)-5-fluoro-1-methyl-1H-pyrazole-4-carboxamide, (15.170) N-(2-tert-butyl-5-methylbenzyl)-N-cyclopropyl-3-(difluoromethyl)-5-fluoro-1-methyl-1H-pyrazole-4-carboxamide, (15.171) N-[5-chloro-2-(trifluoromethyl)benzyl]-N-cyclopropyl-3-(difluoromethyl)-5-fluoro-1-methyl-1H-pyrazole-4-carboxamide, (15.172) N-cyclopropyl-3-(difluoromethyl)-5-fluoro-1-methyl-N-[5-methyl-2-(trifluoromethyl)benzyl]-1H-pyrazole-4-carboxamide, (15.173) N-[2-chloro-6-(trifluoromethyl)benzyl]-N-cyclopropyl-3-(difluoromethyl)-5-fluoro-1-methyl-1H-pyrazole-4-carboxamide, (15.174) N-[3-chloro-2-fluoro-6-(trifluoromethyl)benzyl]-N-cyclopropyl-3-(difluoromethyl)-5-fluoro-1-methyl-1H-pyrazole-4-carboxamide, (15.175) N-cyclopropyl-3-(difluoromethyl)-N-(2-ethyl-4,5-dimethylbenzyl)-5-fluoro-1-methyl-1H-pyrazole-4-carboxamide, (15.176) N-cyclopropyl-3-(difluoromethyl)-5-fluoro-N-(2-isopropylbenzyl)-1-methyl-1H-pyrazol-4-carbothioamide, (15.177) 3-(difluoromethyl)-N-(7-fluoro-1,1,3-trimethyl-2,3-dihydro-1H-inden-4-yl)-1-methyl-1H-pyrazole-4-carboxamide, (15.178) 3-(difluoromethyl)-N-[(3R)-7-fluoro-1,1,3-trimethyl-2,3-dihydro-1H-inden-4-yl]-1-methyl-1H-pyrazole-4-carboxamide, (15.179) 3-(difluoromethyl)-N-[(3 S)-7-fluoro-1,1,3-trimethyl-2,3-dihydro-1H-inden-4-yl]-1-methyl-1H-pyrazole-4-carboxamide, (15.180) N′-(2,5-dimethyl-4-phenoxyphenyl)-N-ethyl-N-methylimidoformamide, (15.181) N′-{4-[(4,5-dichloro-1,3-thiazol-2-yl)oxy]-2,5-dimethylphenyl}-N-ethyl-N-methylimidoformamide, (15.182) N-(4-chloro-2,6-difluorophenyl)-4-(2-chloro-4-fluorophenyl)-1,3-dimethyl-1H-pyrazole-5-amine. All the mixing components mentioned in classes (1) to (15), as the case may be, may form salts with suitable bases or acids if they are capable of doing so on the basis of their functional groups.

### Biological Pesticides as Mixing Components

The compounds of the formula (I) can be combined with biological pesticides.

Biological pesticides include especially bacteria, fungi, yeasts, plant extracts and products formed by microorganisms, including proteins and secondary metabolites.

Biological pesticides include bacteria such as spore-forming bacteria, root-colonizing bacteria and bacteria which act as biological insecticides, fungicides or nematicides.

Examples of such bacteria which are used or can be used as biological pesticides are:

Bacillus amyloliquefaciens, strain FZB42 (DSM 231179), or Bacillus cereus, in particular B. cereus strain CNCM 1-1562 or Bacillus firmus, strain 1-1582 (Accession number CNCM 1-1582) or Bacillus pumilus, in particular strain GB34 (Accession No. ATCC 700814) and strain QST2808 (Accession No. NRRL B-30087), or Bacillus subtilis, in particular strain GB03 (Accession No. ATCC SD-1397), or Bacillus subtilis strain QST713 (Accession No. NRRL B-21661) or Bacillus subtilis strain OST 30002 (Accession No. NRRL B-50421) Bacillus thuringiensis, in particular B. thuringiensis subspecies israelensis (serotype H-14), strain AM65-52 (Accession No. ATCC 1276), or B. thuringiensis subsp. aizawai, in particular strain ABTS-1857 (SD-1372), or B. thuringiensis subsp. kurstaki strain HD-1, or B. thuringiensis subsp. tenebrionis strain NB 176 (SD-5428), Pasteuria penetrans, Pasteuria spp. (Rotylenchulus reniformis nematode)-PR3 (Accession Number ATCC SD-5834), Streptomyces microflavus strain AQ6121 (=QRD 31.013, NRRL B-50550), Streptomyces galbus strain AQ 6047 (Accession Number NRRL 30232).

Examples of fungi and yeasts which are used or can be used as biological pesticides are:

Beauveria bassiana, in particular strain ATCC 74040, Coniothyrium minitans, in particular strain CON/M/91-8 (Accession No. DSM-9660), Lecanicillium spp., in particular strain HRO LEC 12, Lecanicillium lecanii, (formerly known as Verticillium lecanii), in particular strain KV01, Metarhizium anisopliae, in particular strain F52 (DSM3884/ATCC 90448), Metschnikowia fructicola, in particular strain NRRL Y-30752, Paecilomyces fumosoroseus (now: Isaria fumosorosea), in particular strain IFPC 200613, or strain Apopka 97 (Accession No. ATCC 20874), Paecilomyces lilacinus, in particular P. lilacinus strain 251 (AGAL 89/030550), Talaromyces flavus, in particular strain V117b, Trichoderma atroviride, in particular strain SC1 (Accession Number CBS 122089), Trichoderma harzianum, in particular T. harzianum rifai T39. (Accession Number CNCM 1-952).

Examples of viruses which are used or can be used as biological pesticides are:

Adoxophyes orana (summer fruit tortrix) granulosis virus (GV), Cydia pomonella (codling moth) granulosis virus (GV), Helicoverpa armigera (cotton bollworm) nuclear polyhedrosis virus (NPV), Spodoptera exigua (beet armyworm) mNPV, Spodoptera frugiperda (fall armyworm) mNPV, Spodoptera littoralis (African cotton leafworm) NPV.

Also included are bacteria and fungi which are added as ‘inoculant’ to plants or plant parts or plant organs and which, by virtue of their particular properties, promote plant growth and plant health. Examples include: Agrobacterium spp., Azorhizobium caulinodans, Azospirillum spp., Azotobacter spp., Bradyrhizobium spp., Burkholderia spp., especially Burkholderia cepacia (formerly known as Pseudomonas cepacia), Gigaspora spp., or Gigaspora monosporum, Glomus spp., Laccaria spp., Lactobacillus buchneri, Paraglomus spp., Pisolithus tinctorus, Pseudomonas spp., Rhizobium spp., especially Rhizobium trifolii, Rhizopogon spp., Scleroderma spp., Suillus spp., Streptomyces spp.

Examples of plant extracts and products formed by microorganisms, including proteins and secondary metabolites, which are used or can be used as biological pesticides are: Allium sativum, Artemisia absinthium, azadirachtin, Biokeeper WP, Cassia nigricans, Celastrus angulatus, Chenopodium anthelminticum, chitin, Armour-Zen, Dryopteris filix-mas, Equisetum arvense, Fortune Aza, Fungastop, Heads Up (Chenopodium quinoa saponin extract), pyrethrum/pyrethrins, Quassia amara, Quercus, Quillaja, Regalia, “Requiem™ Insecticide”, rotenone, ryania/ryanodine, Symphytum officinale, Tanacetum vulgare, thymol, Triact 70, TriCon, Tropaeulum majus, Urtica dioica, Veratrin, Viscum album, Brassicaceae extract, especially oilseed rape powder or mustard powder.

### Safeners as Mixing Components

The compounds of the formula (I) can be combined with safeners, for example benoxacor, cloquintocet (-mexyl), cyometrinil, cyprosulphamide, dichlormid, fenchlorazole (-ethyl), fenclorim, flurazole, fluxofenim, furilazole, isoxadifen (-ethyl), mefenpyr (-diethyl), naphthalic anhydride, oxabetrinil, 2-methoxy-N-({4-[(methylcarbamoyl)amino]phenyl}sulphonyl)benzamide (CAS 129531-12-0), 4-(dichloroacetyl)-1-oxa-4-azaspiro[4.5]decane (CAS 71526-07-3), 2,2,5-trimethyl-3-(dichloroacetyl)-1,3-oxazolidine (CAS 52836-31-4).

### Plants and Plant Parts

All plants and plant parts can be treated in accordance with the invention. Plants are understood here to mean all plants and populations of plants, such as desirable and undesirable wild plants or crop plants (including naturally occurring crop plants), for example cereals (wheat, rice, triticale, barley, rye, oats), maize, soya beans, potatoes, sugar beet, sugar cane, tomatoes, peas and other types of vegetable, cotton, tobacco, oilseed rape, and also fruit plants (with the fruits apples, pears, citrus fruits and grapevines). Crop plants may be plants which can be obtained by conventional breeding and optimization methods or by biotechnological and genetic engineering methods or combinations of these methods, including the transgenic plants and including the plant cultivars which are protectable and non-protectable by plant breeders' rights. Plant parts shall be understood to mean all parts and organs of the plants above and below ground, such as shoot, leaf, flower and root, examples given being leaves, needles, stalks, stems, flowers, fruit bodies, fruits and seeds, and also roots, tubers and rhizomes. Plant parts also include harvested material and vegetative and generative propagation material, for example cuttings, tubers, rhizomes, slips and seeds.

Treatment according to the invention of the plants and plant parts with the compounds of the formula (I) is carried out directly or by allowing the compounds to act on their surroundings, environment or storage space by the customary treatment methods, for example by immersion, spraying, evaporation, fogging, scattering, painting on, injection and, in the case of propagation material, in particular in the case of seeds, also by applying one or more coats.

As already mentioned above, it is possible to treat all plants and their parts according to the invention. In a preferred embodiment, wild plant species and plant cultivars, or those obtained by conventional biological breeding methods, such as crossing or protoplast fusion, and parts thereof, are treated. In a further preferred embodiment, transgenic plants and plant cultivars obtained by genetic engineering methods, if appropriate in combination with conventional methods (genetically modified organisms), and parts thereof are treated. The term “parts” or “parts of plants” or “plant parts” has been explained above. Particular preference is given in accordance with the invention to treating plants of the respective commercially customary cultivars or those that are in use. Plant cultivars are understood to mean plants having new properties (“traits”) and which have been obtained by conventional breeding, by mutagenesis or by recombinant DNA techniques. They may be cultivars, varieties, biotypes or genotypes.

### Transgenic Plants, Seed Treatment and Integration Events

The preferred transgenic plants or plant cultivars (those obtained by genetic engineering) which are to be treated in accordance with the invention include all plants which, through the genetic modification, received genetic material which imparts particular advantageous useful properties (“traits”) to these plants. Examples of such properties are better plant growth, increased tolerance to high or low temperatures, increased tolerance to drought or to levels of water or soil salinity, enhanced flowering performance, easier harvesting, accelerated ripening, higher harvest yields, higher quality and/or higher nutritional value of the harvested products, better storage life and/or processability of the harvested products. Further and particularly emphasized examples of such properties are increased resistance of the plants against animal and microbial pests, such as insects, arachnids, nematodes, mites, slugs and snails owing, for example, to toxins formed in the plants, in particular those produced in the plants by the genetic material from Bacillus thuringiensis (for example by the genes CryIA(a), CryIA(b), CryIA(c), CryIIA, CryIIIA, CryIIIB2, Cry9c Cry2Ab, Cry3Bb and CryIF and also combinations thereof), and also increased resistance of the plants against phytopathogenic fungi, bacteria and/or viruses caused, for example, by systemic acquired resistance (SAR), systemin, phytoalexins, elicitors and resistance genes and correspondingly expressed proteins and toxins, and also increased tolerance of the plants to certain herbicidally active compounds, for example imidazolinones, sulphonylureas, glyphosates or phosphinothricin (for example the “PAT” gene). The genes which impart the desired traits in question may also be present in combinations with one another in the transgenic plants. Examples of transgenic plants include the important crop plants, such as cereals (wheat, rice, triticale, barley, rye, oats), maize, soya beans, potatoes, sugar beet, sugar cane, tomatoes, peas and other types of vegetable, cotton, tobacco, oilseed rape and also fruit plants (with the fruits apples, pears, citrus fruits and grapevines), particular emphasis being given to maize, soya beans, wheat, rice, potatoes, cotton, sugar cane, tobacco and oilseed rape. Properties (“traits”) which are particularly emphasized are the increased resistance of the plants to insects, arachnids, nematodes and slugs and snails.

### Crop Protection—Types of Treatment

The treatment of the plants and plant parts with the compounds of the formula (I) is carried out directly or by action on their surroundings, habitat or storage space using customary treatment methods, for example by dipping, spraying, atomizing, irrigating, evaporating, dusting, fogging, broadcasting, foaming, painting, spreading-on, injecting, watering (drenching), drip irrigating and, in the case of propagation material, in particular in the case of seed, furthermore as a powder for dry seed treatment, a solution for liquid seed treatment, a water-soluble powder for slurry treatment, by incrusting, by coating with one or more coats, etc.

It is furthermore possible to apply the compounds of the formula (I) by the ultra-low volume method or to inject the application form or the compound of the formula (I) itself into the soil.

A preferred direct treatment of the plants is foliar application, i.e. the compounds of the formula (I) are applied to the foliage, where treatment frequency and the application rate should be adjusted according to the level of infestation with the pest in question.

In the case of systemically active compounds, the compounds of the formula (I) also access the plants via the root system. The plants are then treated by the action of the compounds of the formula (I) on the habitat of the plant. This can be accomplished, for example, by drenching, or by mixing into the soil or the nutrient solution, meaning that the locus of the plant (e.g. soil or hydroponic systems) is impregnated with a liquid form of the compounds of the formula (I), or by soil application, meaning that the compounds of the formula (I) are introduced in solid form (e.g. in the form of granules) into the locus of the plants. In the case of paddy rice crops, this can also be accomplished by metering the compound of the formula (I) in a solid application form (for example as granules) into a flooded paddy field.

### Seed Treatment

The control of animal pests by the treatment of the seed of plants has long been known and is the subject of constant improvement. However, the treatment of seed entails a series of problems which cannot always be solved in a satisfactory manner. Thus, it is desirable to develop methods for protecting the seed and the germinating plant which dispense with, or at least reduce considerably, the additional application of pesticides during storage, after sowing or after emergence of the plants. It is additionally desirable to optimize the amount of active compound used so as to provide optimum protection for the seed and the germinating plant from attack by animal pests, but without damage to the plant itself by the active compound used. In particular, methods for the treatment of seed should also take account of the intrinsic insecticidal or nematicidal properties of pest-resistant or -tolerant transgenic plants in order to achieve optimal protection of the seed and the germinating plant with a minimum expenditure of pesticides.

The present invention therefore in particular also relates to a method for the protection of seed and germinating plants, from attack by pests, by treating the seed with one of the compounds of the formula (I). The method according to the invention for protecting seed and germinating plants against attack by pests further comprises a method in which the seed is treated simultaneously in one operation or sequentially with a compound of the formula (I) and a mixing component. It also comprises a method where the seed is treated at different times with a compound of the formula (I) and a mixing component.

The invention likewise relates to the use of the compounds of the formula (I) for the treatment of seed for protecting the seed and the resulting plant from animal pests.

The invention further relates to seed which has been treated with a compound of the formula (I) for protection from animal pests. The invention also relates to seed which has been treated simultaneously with a compound of the formula (I) and a mixing component. The invention further relates to seed which has been treated at different times with a compound of the formula (I) and a mixing component. In the case of seed which has been treated at different times with a compound of the formula (I) and a mixing component, the individual substances may be present on the seed in different layers. In this case, the layers comprising a compound of the formula (I) and mixing components may optionally be separated by an intermediate layer.

The invention also relates to seed in which a compound of the formula (I) and a mixing component have been applied as part of a coating or as a further layer or further layers in addition to a coating.

The invention further relates to seed which, after the treatment with a compound of the formula (I), is subjected to a film-coating process to prevent dust abrasion on the seed.

One of the advantages that occurs when one of the compounds of the formula (I) acts systemically is that the treatment of the seed protects not just the seed itself but also the plants resulting therefrom after emergence against animal pests. In this way, the immediate treatment of the crop at the time of sowing or shortly thereafter can be dispensed with.

A further advantage is that the treatment of the seed with a compound of the formula (I) can enhance germination and emergence of the treated seed.

It is likewise considered to be advantageous that compounds of the formula (I) can especially also be used for transgenic seed.

Furthermore, compounds of the formula (I) can be employed in combination with compositions of signalling technology, leading to better colonization by symbionts such as, for example, rhizobia, mycorrhizae and/or endophytic bacteria or fungi, and/or to optimized nitrogen fixation.

The compounds of the formula (I) are suitable for protection of seed of any plant variety which is used in agriculture, in greenhouses, in forests or in horticulture. More particularly, this includes seed of cereals (for example wheat, barley, rye, millet and oats), maize, cotton, soya beans, rice, potatoes, sunflowers, coffee, tobacco, canola, oilseed rape, beet (for example sugar beet and fodder beet), peanuts, vegetables (for example tomatoes, cucumbers, beans, cruciferous vegetables, onions and lettuce), fruit plants, lawns and ornamental plants. Of particular significance is the treatment of the seed of cereals (such as wheat, barley, rye and oats), maize, soya bean, cotton, canola, oilseed rape and rice.

As already mentioned above, the treatment of transgenic seed with a compound of the formula (I) is also of particular importance. This involves the seed of plants which generally contain at least one heterologous gene which controls the expression of a polypeptide having insecticidal and/or nematicidal properties in particular. The heterologous genes in transgenic seed may originate from microorganisms such as Bacillus, Rhizobium, Pseudomonas, Serratia, Trichoderma, Clavibacter, Glomus or Gliocladium. The present invention is particularly suitable for the treatment of transgenic seed containing at least one heterologous gene originating from Bacillus sp. The heterologous gene is more preferably derived from Bacillus thuringiensis.

In the context of the present invention, the compound of the formula (I) is applied to the seed. The seed is preferably treated in a state in which it is sufficiently stable for no damage to occur in the course of treatment. In general, the seed can be treated at any time between harvest and sowing. It is customary to use seed which has been separated from the plant and freed from cobs, shells, stalks, coats, hairs or the flesh of the fruits. Thus, for example, it is possible to use seed which has been harvested, cleaned and dried down to a moisture content which allows storage. Alternatively, it is also possible to use seed which, after drying, has been treated with, for example, water and then dried again, for example priming. In the case of rice seed, it is also possible to use seed which, for example, has been pre-swollen in water up to a particular stage (pigeon breast stage), which leads to better germination and to more homogeneous emergence.

When treating the seed, care must generally be taken that the amount of the compound of the formula (I) applied to the seed and/or the amount of further additives is chosen in such a way that the germination of the seed is not adversely affected, or that the resulting plant is not damaged. This has to be ensured particularly in the case of active compounds which can exhibit phytotoxic effects at certain application rates.

In general, the compounds of the formula (I) are applied to the seed in a suitable formulation. Suitable formulations and processes for seed treatment are known to the person skilled in the art.

The compounds of the formula (I) can be converted to the customary seed-dressing formulations, such as solutions, emulsions, suspensions, powders, foams, slurries or other coating compositions for seed, and also ULV formulations.

These formulations are produced in a known manner, by mixing compounds of the formula (I) with customary additives, for example customary extenders and solvents or diluents, dyes, wetters, dispersants, emulsifiers, antifoams, preservatives, secondary thickeners, stickers, gibberellins and also water.

Useful dyes which may be present in the seed-dressing formulations usable in accordance with the invention are all dyes which are customary for such purposes. It is possible to use either pigments, which are sparingly soluble in water, or dyes, which are soluble in water. Examples include the dyes known by the names Rhodamine B, C.I. Pigment Red 112 and C.I. Solvent Red 1.

Useful wetting agents which may be present in the seed-dressing formulations usable in accordance with the invention are all substances which promote wetting and which are customary for the formulation of active agrochemical compounds. Preference is given to using alkyl naphthalenesulphonates, such as diisopropyl or diisobutyl naphthalenesulphonates.

Suitable dispersants and/or emulsifiers which may be present in the seed-dressing formulations usable in accordance with the invention are all nonionic, anionic and cationic dispersants customary for the formulation of active agrochemical compounds. Preference is given to using nonionic or anionic dispersants or mixtures of nonionic or anionic dispersants. Suitable nonionic dispersants include in particular ethylene oxide/propylene oxide block polymers, alkylphenol polyglycol ethers and tristyrylphenol polyglycol ethers, and the phosphated or sulphated derivatives thereof. Suitable anionic dispersants are especially lignosulphonates, polyacrylic acid salts and arylsulphonate/formaldehyde condensates.

Antifoams which may be present in the seed-dressing formulations usable in accordance with the invention are all foam-inhibiting substances customary for formulation of active agrochemical compounds. Silicone antifoams and magnesium stearate can be used with preference.

Preservatives which may be present in the seed-dressing formulations usable in accordance with the invention are all substances usable for such purposes in agrochemical compositions. Examples include dichlorophene and benzyl alcohol hemiformal.

Secondary thickeners which may be present in the seed-dressing formulations usable in accordance with the invention are all substances which can be used for such purposes in agrochemical compositions. Preferred examples include cellulose derivatives, acrylic acid derivatives, xanthan, modified clays and finely divided silica.

Useful stickers which may be present in the seed-dressing formulations usable in accordance with the invention are all customary binders usable in seed-dressing products. Preferred examples include polyvinylpyrrolidone, polyvinyl acetate, polyvinyl alcohol and tylose.

Gibberellins which may be present in the seed-dressing formulations usable in accordance with the invention are preferably the gibberellins A1, A3 (=gibberellic acid), A4 and A7; particular preference is given to using gibberellic acid. The gibberellins are known (cf. R. Wegler “Chemie der Pflanzenschutz-und Schaidlingsbekimpfungsmittel”, vol. 2, Springer Verlag, 1970, pp. 401-412).

The seed-dressing formulations usable in accordance with the invention can be used to treat a wide variety of different kinds of seed, either directly or after prior dilution with water. For instance, the concentrates or the preparations obtainable therefrom by dilution with water can be used to dress the seed of cereals, such as wheat, barley, rye, oats, and triticale, and also the seed of maize, rice, oilseed rape, peas, beans, cotton, sunflowers, soya beans and beets, or else a wide variety of different vegetable seed. The seed-dressing formulations usable in accordance with the invention, or the dilute use forms thereof, can also be used to dress seed of transgenic plants.

For treatment of seed with the seed-dressing formulations usable in accordance with the invention, or the use forms prepared therefrom, all mixing units usable customarily for the seed dressing are useful. Specifically, the procedure in seed dressing is to place the seed into a mixer in batchwise or continuous operation, to add the particular desired amount of seed-dressing formulations, either as such or after prior dilution with water, and to mix until the formulation is distributed homogeneously on the seed. If appropriate, this is followed by a drying operation.

The application rate of the seed-dressing formulations usable in accordance with the invention can be varied within a relatively wide range. It is guided by the particular content of the compounds of the formula (I) in the formulations and by the seed. The application rates of the compound of the formula (I) are generally between 0.001 and 50 g per kilogram of seed, preferably between 0.01 and 15 g per kilogram of seed.

### Animal Health

In the field of animal health, i.e. in the field of veterinary medicine, the compounds of the formula (I) are active against animal parasites, in particular ectoparasites or endoparasites. The term “endoparasites” includes especially helminths and protozoans, such as coccidia. Ectoparasites are typically and preferably arthropods, especially insects and acarids.

In the field of veterinary medicine, the compounds of the formula (I) having favourable toxicity to warm-blooded species are suitable for controlling parasites which occur in animal breeding and animal husbandry in livestock, breeding animals, zoo animals, laboratory animals, experimental animals and domestic animals. They are active against all or specific stages of development of the parasites.

Agricultural livestock include, for example, mammals such as sheep, goats, horses, donkeys, camels, buffalo, rabbits, reindeer, fallow deer, and particularly cattle and pigs; poultry such as turkeys, ducks, geese, and particularly chickens; fish and crustaceans, for example in aquaculture, and also insects such as bees.

Domestic animals include, for example, mammals, such as hamsters, guinea pigs, rats, mice, chinchillas, ferrets, and particularly dogs, cats, caged birds, reptiles, amphibians and aquarium fish.

In a preferred embodiment, the compounds of the formula (I) are administered to mammals.

In another preferred embodiment, the compounds of the formula (I) are administered to birds, namely caged birds and particularly poultry.

Use of the compounds of the formula (I) for the control of animal parasites is intended to reduce or prevent illness, cases of death and reductions in performance (in the case of meat, milk, wool, hides, eggs, honey and the like), such that more economical and simpler animal keeping is enabled and better animal well-being is achievable.

In relation to the field of animal health, the term “control” or “controlling” means that the compounds of the formula (I) are effective in reducing the incidence of the particular parasite in an animal infected with such parasites to an innocuous degree. More specifically, “controlling” in the present context means that the compound of the formula (I) can kill the respective parasite, inhibit its growth, or inhibit its proliferation.

Arthropods include:

from the order of the Anoplurida, for example Haematopinus spp., Linognathus spp., Pediculus spp., Phtirus spp., Solenopotes spp.; from the order of the Mallophagida and the suborders Amblycerina and Ischnocerina, for example Trimenopon spp., Menopon spp., Trinoton spp., Bovicola spp., Wemeckiella spp., Lepikentron spp., Damalina spp., Trichodectes spp., Felicola spp.; from the order of the Diptera and the suborders Nematocerina and Brachycerina, for example Aedes spp., Anopheles spp., Culex spp., Simulium spp., Eusimulium spp., Phlebotomus spp., Lutzomyia spp., Culicoides spp., Chrysops spp., Odagmia spp., Wilhelmia spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp., Melophagus spp., Rhinoestrus spp., Tipula spp.; from the order of the Siphonapterida, for example Pulex spp., Ctenocephalides spp., Tunga spp., Xenopsylla spp., Ceratophyllus spp.;
from the order of the Heteropterida, for example Cimex spp., Triatoma spp., Rhodnius spp., Panstrongylus spp.; and also nuisance and hygiene pests from the order of the Blattarida.
Arthropods further include:
from the subclass of the Acari (Acarina) and the order of the Metastigmata, for example from the family of Argasidae like Argas spp., Omithodorus spp., Otobius spp., from the family of Ixodidae like Ixodes spp., Amblyomma spp., Rhipicephalus (Boophilus) spp., Dermacentor spp., Haemophysalis spp., Hyalomma spp., Rhipicephalus spp. (the original genus of multi-host ticks); from the order of Mesostigmata like Dermanyssus spp., Omithonyssus spp., Pneumonyssus spp., Raillietia spp., Pneumonyssus spp., Sternostoma spp., Varroa spp., Acarapis spp.; from the order of the Actinedida (Prostigmata), for example Acarapis spp., Cheyletiella spp., Omithocheyletia spp., Myobia spp., Psorergates spp., Demodex spp., Trombicula spp., Neotrombiculla spp., Listrophorus spp.; and from the order of the Acaridida (Astigmata), for example Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., Laminosioptes spp.

Parasitic Protozoa include:

Mastigophora (Flagellata), for example Trypanosomatidae, for example, Trypanosoma b. brucei, T.b. gambiense, T.b. rhodesiense, T. congolense, T. cruzi, T. evansi, T. equinum, T. lewisi, T. percae, T. simiae, T. vivax, Leishmania brasiliensis, L. donovani, L. tropica, for example Trichomonadidae, for example, Giardia lamblia, G. canis.
Sarcomastigophora (Rhizopoda) such as Entamoebidae, for example, Entamoeba histolytica, Hartmanellidae, for example, Acanthamoeba sp., Harmanella sp.
Apicomplexa (Sporozoa) such as Eimeridae, for example, Eimeria acervulina, E. adenoides, E. alabamensis, E. anatis, E. anserina, E. arloingi, E. ashata, E. aubumensis, E. bovis, E. brunetti, E. canis, E. chinchillae, E. clupearum, E. columbae, E. contorta, E. crandalis, E. debliecki, E. dispersa, E. ellipsoidales, E. falciformis, E. faurei, E. flavescens, E. gallopavonis, E. hagani, E. intestinalis, E. iroquoina, E. irresidua, E. labbeana, E. leucarti, E. magna, E. maxima, E. media, E. meleagridis, E. meleagrimitis, E. mitis, E. necatrix, E. ninakohlyakimovae, E. ovis, E. parva, E. pavonis, E. perforans, E. phasani, E. piriformis, E. praecox, E. residua, E. scabra, E. spec., E. stiedai, E. suis, E. tenella, E. truncata, E. truttae, E. zuemii, Globidium spec., Isospora belli, I. canis, I. felis, I. ohioensis, I. rivolta, I. spec., I. suis, Cystisospora spec., Cryptosporidium spec., in particular C. parvum; such as Toxoplasmadidae, for example Toxoplasma gondii, Hammondia heydomii, Neospora caninum, Besnoitia besnoitii; such as Sarcocystidae, for example Sarcocystis bovicanis, S. bovihominis, S. ovicanis, S. ovifelis, S. neurona, S. spec., S. suihominis, such as Leucozoidae, for example Leucozytozoon simondi, such as Plasmodiidae, for example Plasmodium berghei, P. falciparum, P. malariae, P. ovale, P. vivax, P. spec., such as Piroplasmea, for example Babesia argentina, B. bovis, B. canis, B. spec., Theileria parva, Theileria spec., such as Adeleina, for example Hepatozoon canis, H. spec.

Pathogenic endoparasites which are helminths include Platyhelmintha (e.g. Monogenea, cestodes and trematodes), nematodes, Acanthocephala, and Pentastoma. These include:

Monogenea: for example: Gyrodactylus spp., Dactylogyrus spp., Polystoma spp; Cestodes: from the order of the Pseudophyllidea for example: Diphyllobothrium spp., Spirometra spp., Schistocephalus spp., Ligula spp., Bothridium spp., Diplogonoporus spp;

from the order of the Cyclophyllida, for example: Mesocestoides spp., Anoplocephala spp., Paranoplocephala spp., Moniezia spp., Thysanosoma spp., Thysaniezia spp., Avitellina spp., Stilesia spp., Cittotaenia spp., Andyra spp., Bertiella spp., Taenia spp., Echinococcus spp., Hydatigera spp., Davainea spp., Raillietina spp., Hymenolepis spp., Echinolepis spp., Echinocotyle spp., Diorchis spp., Dipylidium spp., Joyeuxiella spp., Diplopylidium spp;

Trematodes: from the class of the Digenea, for example: Diplostomum spp., Posthodiplostomum spp., Schistosoma spp., Trichobilharzia spp., Ornithobilharzia spp., Austrobilharzia spp., Gigantobilharzia spp., Leucochloridium spp., Brachylaima spp., Echinostoma spp., Echinoparyphium spp., Echinochasmus spp., Hypoderaeum spp., Fasciola spp., Fasciolides spp., Fasciolopsis spp., Cyclocoelum spp., Typhlocoelum spp., Paramphistomum spp., Calicophoron spp., Cotylophoron spp., Gigantocotyle spp., Fischoederius spp., Gastrothylacus spp., Notocotylus spp., Catatropis spp., Plagiorchis spp., Prosthogonimus spp., Dicrocoelium spp., Eurytrema spp., Troglotrema spp., Paragonimus spp., Collyriclum spp., Nanophyetus spp., Opisthorchis spp., Clonorchis spp., Metorchis spp., Heterophyes spp., Metagonimus spp.; Nematodes: Trichinellida, for example: Trichuris spp., Capillaria spp., Paracapillaria spp., Eucoleus spp., Trichomosoides spp., Trichinella spp.;

From the order of the Tylenchida, for example: Micronema spp., Strongyloides spp.;

from the order of the Rhabditida, for example: Strongylus spp., Triodontophorus spp., Oesophagodontus spp., Trichonema spp., Gyalocephalus spp., Cylindropharynx spp., Poteriostomum spp., Cyclococercus spp., Cylicostephanus spp., Oesophagostomum spp., Chabertia spp., Stephanurus spp., Ancylostoma spp., Uncinaria spp., Necator spp., Bunostomum spp., Globocephalus spp., Syngamus spp., Cyathostoma spp., Metastrongylus spp., Dictyocaulus spp., Muellerius spp., Protostrongylus spp., Neostrongylus spp., Cystocaulus spp., Pneumostrongylus spp., Spicocaulus spp., Elaphostrongylus spp., Parelaphostrongylus spp., Crenosoma spp., Paracrenosoma spp., Oslerus spp., Angiostrongylus spp., Aelurostrongylus spp., Filaroides spp., Parafilaroides spp., Trichostrongylus spp., Haemonchus spp., Ostertagia spp., Teladorsagia spp., Marshallagia spp., Cooperia spp., Nippostrongylus spp., Heligmosomoides spp., Nematodirus spp., Hyostrongylus spp., Obeliscoides spp., Amidostomum spp., Ollulanus spp.; From the order of the Spirurida, for example: Oxyuris spp., Enterobius spp., Passalurus spp., Syphacia spp., Aspiculuris spp., Heterakis spp.; Ascaris spp., Toxascaris spp., Toxocara spp., Baylisascaris spp., Parascaris spp., Anisakis spp., Ascaridia spp.; Gnathostoma spp., Physaloptera spp., Thelazia spp., Gongylonema spp., Habronema spp., Parabronema spp., Draschia spp., Dracunculus spp.; Stephanofilaria spp., Parafilaria spp., Setaria spp., Loa spp., Dirofilaria spp., Litomosoides spp., Brugia spp., Wuchereria spp., Onchocerca spp., Spirocerca spp.;

Acanthocephala: from the order of the Oligacanthorhynchida, for example: Macracanthorhynchus spp., Prosthenorchis spp.; from the order of the Polymorphida, for example: Filicollis spp.; from the order of the Moniliformida, for example: Moniliformis spp.;

From the order of the Echinorhynchida, for example, Acanthocephalus spp., Echinorhynchus spp., Leptorhynchoides spp.

Pentastoma: from the order of the Porocephalida, for example, Linguatula spp.

In the veterinary field and in animal keeping, the compounds of the formula (I) are administered by methods generally known in the art, such as via the enteral, parenteral, dermal or nasal route in the form of suitable preparations. Administration may be prophylactic or therapeutic.

Thus, one embodiment of the present invention refers to the use of a compound of the formula (I) as a medicament.

A further aspect refers to the use of a compound of the formula (I) as an antiendoparasitic agent, in particular a helminthicidal agent or antiprotozoic agent. Compounds of the formula (I) are suitable for use as an antiendoparasitic agent, especially as a helminthicidal agent or antiprotozoic agent, for example in animal breeding, in animal husbandry, in animal houses and in the hygiene sector.

A further aspect in turn relates to the use of a compound of the formula (I) as an antiectoparasitic, in particular an arthropodicide such as an insecticide or an acaricide. A further aspect relates to the use of a compound of the formula (I) as an antiectoparasitic, in particular an arthropodicide such as an insecticide or an acaricide, for example in animal husbandry, in animal breeding, in animal houses or in the hygiene sector.

### Anthelmintic Mixing Partners

The following anthelmintic mixing partners may be mentioned by way of example:

Anthelmintically active compounds including trematicidally and cestocidally active compounds:

from the class of the macrocyclic lactones, for example: abamectin, doramectin, emamectin, eprinomectin, ivermectin, milbemycin, moxidectin, nemadectin, selamectin;
from the class of the benzimidazoles and probenzimidazoles, for example: albendazole, albendazole sulphoxide, cambendazole, cyclobendazole, febantel, fenbendazole, flubendazole, mebendazole, netobimin, oxfendazole, oxibendazole, parbendazole, thiabendazole, thiophanate, triclabendazole;
from the class of the cyclooctadepsipeptides, for example: emodepside, PF1022;
from the class of the aminoacetonitrile derivates, for example: monepantel;
from the class of the tetrahydropyrimidines, for example: morantel, pyrantel, oxantel;
from the class of the imidazothiazoles, for example: butamisole, levamisole, tetramisole;
from the class of the salicylanilides, for example: bromoxanide, brotianide, clioxanide, closantel,
niclosamide, oxyclozanide, rafoxanide, tribromsalane;
from the class of the paraherquamides, for example: derquantel, paraherquamide;
from the class of the aminophenylamidines, for example: amidantel, deacylated amidantel (dAMD),
tribendimidine;
from the class of the organophosphates, for example: coumaphos, crufomate, dichlorvos, haloxon, naphthalofos, trichlorfon;
from the class of the substituted phenols, for example: bithionol, disophenol, hexachlorophene, niclofolan, meniclopholan, nitroxynil;
from the class of the piperazinones, for example: praziquantel, epsiprantel;
from other diverse classes, for example: amoscanate, bephenium, bunamidine, clonazepam, clorsulon, diamfenetide, dichlorophen, diethylcarbamazine, emetine, Hetolin, hycanthone, lucanthone, miracil, mirasan, niclosamide, niridazole, nitroxynil, nitroscanate, oltipraz, omphalotin, oxamniquine, paromomycin, piperazine, resorantel.

### Vector Control

The compounds of the formula (I) can also be used in vector control. In the context of the present invention, a vector is an arthropod, especially an insect or arachnid, capable of transmitting pathogens, for example, viruses, worms, single-cell organisms and bacteria, from a reservoir (plant, animal, human, etc.) to a host. The pathogens can be transmitted either mechanically (for example trachoma by non-stinging flies) to a host or after injection (for example malaria parasites by mosquitoes) into a host.

Examples of vectors and the diseases or pathogens they transmit are:

### 1) Mosquitoes

Anopheles: malaria, filariasis;
Culex: Japanese encephalitis, filariasis, other viral diseases, transmission of worms;
Aedes: yellow fever, dengue fever, filariasis, other viral diseases;
Simulidae: transmission of worms, in particular Onchocerca volvulus;
   2) Lice: skin infections, epidemic typhus;
   3) Fleas: plague, endemic typhus;
   4) Flies: sleeping sickness (trypanosomiasis); cholera, other bacterial diseases;
   5) Mites: acariosis, epidemic typhus, rickettsialpox, tularaemia, Saint Louis encephalitis, tick-borne encephalitis (TBE), Crimean-Congo haemorrhagic fever, borreliosis;
   6) Ticks: borellioses such as Borrelia duttoni, tick-borne encephalitis, Q fever (Coxiella burnetii), babesioses (Babesia canis canis).

Examples of vectors in the context of the present invention are insects, for example aphids, flies, leafhoppers or thrips, which can transmit plant viruses to plants. Other vectors capable of transmitting plant viruses are spider mites, lice, beetles and nematodes.

Further examples of vectors in the context of the present invention are insects and arachnids such as mosquitoes, especially of the genera Aedes, Anopheles, for example A. gambiae, A. arabiensis, A. funestus, A. dirs (malaria) and Culex, lice, fleas, flies, mites and ticks, which can transmit pathogens to animals and/or humans.

Vector control is also possible if the compounds of the formula (I) are resistance-breaking.

Compounds of the formula (I) are suitable for use in the prevention of diseases and/or pathogens transmitted by vectors. Thus, a further aspect of the present invention is the use of compounds of the formula (I) for vector control, for example in agriculture, in horticulture, in forestry, in gardens and in leisure facilities, and also in the protection of materials and stored products.

### Protection of Industrial Materials

The compounds of the formula (I) are suitable for protecting industrial materials against attack or destruction by insects, for example from the orders Coleoptera, Hymenoptera, Isoptera, Lepidoptera, Psocoptera and Zygentoma.

Industrial materials in the present context are understood to mean inanimate materials, such as preferably plastics, adhesives, sizes, papers and cards, leather, wood, processed wood products and coating compositions. The use of the invention for protection of wood is particularly preferred.

In a further embodiment, the compounds of the formula (I) are used together with at least one further insecticide and/or at least one fungicide.

In a further embodiment, the compounds of the formula (I) are present as a ready-to-use pesticide, i.e. they can be applied to the material in question without further modifications. Suitable further insecticides or fungicides are in particular those mentioned above.

Surprisingly, it has also been found that the compounds of the formula (I) can be employed for protecting objects which come into contact with saltwater or brackish water, in particular hulls, screens, nets, buildings, moorings and signalling systems, against fouling. It is equally possible to use the compounds of the formula (I), alone or in combinations with other active compounds, as antifouling agents.

### Control of Animal Pests in the Hygiene Sector

The compounds of the formula (I) are suitable for controlling animal pests in the hygiene sector. More particularly, the invention can be used in the domestic sector, in the hygiene sector and in the protection of stored products, particularly for control of insects, arachnids and mites encountered in enclosed spaces, for example dwellings, factory halls, offices, vehicle cabins. For controlling animal pests, the compounds of the formula (I) are used alone or in combination with other active compounds and/or auxiliaries. They are preferably used in domestic insecticide products. The compounds of the formula (I) are effective against sensitive and resistant species, and against all developmental stages.

These pests include, for example, pests from the class Arachnida, from the orders Scorpiones, Araneae and Opiliones, from the classes Chilopoda and Diplopoda, from the class Insecta the order Blattodea, from the orders Coleoptera, Dermaptera, Diptera, Heteroptera, Hymenoptera, Isoptera, Lepidoptera, Phthiraptera, Psocoptera, Saltatoria or Orthoptera, Siphonaptera and Zygentoma and from the class Malacostraca the order Isopoda.

Application is effected, for example, in aerosols, unpressurized spray products, for example pump and atomizer sprays, automatic fogging systems, foggers, foams, gels, evaporator products with evaporator tablets made of cellulose or plastic, liquid evaporators, gel and membrane evaporators, propeller-driven evaporators, energy-free, or passive, evaporation systems, moth papers, moth bags and moth gels, as granules or dusts, in baits for spreading or in bait stations.

### EXAMPLES

The preparation and use examples which follow illustrate the invention without limiting it. The products were characterized by 1H-NMR or 13C-NMR spectroscopy and/or LC-MS (liquid chromatography mass spectrometry).

The logP values were determined in accordance with EEC Directive 79/831 Annex V.A8 by HPLC (High Performance Liquid Chromatography) using reversed-phase columns (C 18) by the following methods:

^{[a]} The LC-MS determination in the acidic range was effected at pH 2.7 with 0.1% aqueous formic acid and acetonitrile (contains 0.1% formic acid) as eluents; linear gradient from 10% acetonitrile to 95% acetonitrile. Called logP (HCOOH) in the table.

^{[b]} LC-MS determination in the neutral range was effected at pH 7.8 with 0.001 molar aqueous ammonium hydrogencarbonate solution and acetonitrile as eluents; linear gradient from 10% acetonitrile to 95% acetonitrile. Called logP (neutral) in the table.

Calibration was effected with unbranched alkan-2-ones (having 3 to 16 carbon atoms) with known logP values (logP values determined on the basis of the retention times by linear interpolation between two successive alkanones).

The lambda-max values were determined in the maxima of the chromatographic signals using the UV spectra from 200 nm to 400 nm.

The NMR data for selected examples are listed in conventional form (8 values, number of hydrogen atoms, multiplet splitting).

The solvent in which the NMR spectrum was recorded is reported in each case.

### Preparation Examples

### Preparation Example 1: 6-{2-Fluoro-4-methyl-5-[(2,2,2-trifluoroethyl)sulphinyl]phenyl}-1,3-dimethylpyrimidine-2,4(1H,3H)-dione (I-1)

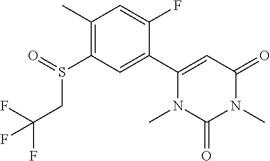 [SMILES: Cc1cc(F)c(-c2cc(=O)n(C)c(=O)n2C)cc1S(=O)CC(F)(F)F]

At 0° C., 75 mg (0.207 mmol) of 6-{2-fluoro-4-methyl-5-[(2,2,2-trifluoroethyl)sulphanyl]phenyl}-1,3-dimethylpyrimidine-2,4(1H,3H)-dione (1-2) were initially charged in 5 ml of acetic acid. After the addition of catalytic amounts of sodium tungstate at 0-4° C., 185 mg (0.159 mmol) of 3% aqueous hydrogen peroxide solution were added thereto in portions and the reaction mixture was stirred at room temperature for 24 h. After the addition of a 33% strength aqueous bisulphite solution, the mixture was extracted twice with dichloromethane. The combined organic phases were washed with water, dried over sodium sulphate and filtered. After removal of the solvent under reduced pressure, the residue was purified by column chromatography on a 10 g SNAP column (Biotage) using the mobile phase ethyl acetate/cyclohexane (gradient 20-76% ethyl acetate). This gave 31 mg of product as a white solid (39.6% of theory, purity 91.6% according to LC/MS).

1H-NMR (D6-DMSO) δ ppm: 7.94-7.92 (m, 1H), 7.53-7.51 (m, 1H), 5.93 (broad, 1H), 4.19 (broad, 2H), 3.23 (s, 3H), 3.09 (s, 3H), 2.46 (s, 3H)

logP (HCOOH): 1.77 logP (neutral): 1.75

### Preparation Example 2: 6-{2-Fluoro-4-methyl-5-[(2,2,2-trifluoroethyl)sulphanyl]phenyl}-1,3-dimethylpyrimidine-2,4(1H,3H)-dione (I-2)

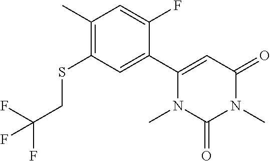 [SMILES: Cc1cc(F)c(-c2cc(=O)n(C)c(=O)n2C)cc1SCC(F)(F)F]

At room temperature, 100 mg (0.573 mmol) of 6-chloro-1,3-dimethylpyrimidine-2,4(1H,3H)-dione and 55 mg (0.078 mmol) of bis(triphenylphosphine)palladium(II) dichloride were stirred in 20 ml of dioxane for 2 h. 195 mg (0.728 mmol) of {2-fluoro-4-methyl-5-[(2,2,2-trifluoroethyl)sulphanyl]phenyl}boric acid and 3 ml of 2 molar sodium carbonate solution were then added and the mixture was heated under reflux for 18 h. After cooling, water was added and the reaction mixture was extracted with dichloromethane. After filtration through a layer of silica gel, the combined organic phases were dried over MgSO₄ and freed of the solvent under reduced pressure. The residue was purified by column chromatography on a 10 g SNAP column (Biotage) using the mobile phase ethyl acetate/cyclohexane (gradient 5-61% ethyl acetate). This gave 46 g of product as a light-yellow viscous oil (22.2% of theory, purity according to LC/MS neutral 100%).

1H-NMR (D₆-DMSO) δ ppm: 7.68-7.66 (m, 1H), 7.42-7.39 (m, 1H), 5.77 (s, 1H), 4.06-3.99 (q, 2H), 3.23 (s, 3H), 3.06 (s, 3H), 2.44 (s, 3H)

logP (HCOOH): 2.86 logP (neutral): 2.74

### Preparation Example 3: 3-Ethyl-5-fluoro-6-{2-fluoro-4-methyl-5-[(2,2,2-trifluoroethyl)-sulphanyl]phenyl}-1-methylpyrimidine-2,4(1H,3H)-dione (1-3)

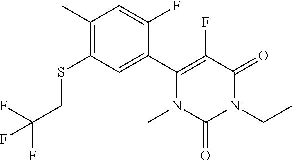 [SMILES: CCn1c(=O)c(F)c(-c2cc(SCC(F)(F)F)c(C)cc2F)n(C)c1=O]

At room temperature, 340 mg (1.65 mmol) of 6-chloro-3-ethyl-5-fluoro-1-methylpyrimidine-2,4(1H,3H)-dione (II-01) and 170 mg (0.242 mmol) of bis(triphenylphosphine)palladium(II) dichloride were stirred in 20 ml of dioxane for 2 h. 530 mg (1.98 mmol) of {2-fluoro-4-methyl-5-[(2,2,2-trifluoroethyl)-sulphanyl]phenyl}boric acid and 9 ml of 2-molar sodium carbonate solution were added, and the mixture was heated under reflux for 18 h. After cooling, water was added and the reaction mixture was extracted with dichloromethane. The combined organic phases were, after filtration through a layer of silica gel, dried over MgSO₄ and freed from the solvent under reduced pressure. The residue was purified by column chromatography using MPLC on RP(C-18) with water/acetonitrile. This gave 26 mg of product as a white solid (4% of theory, purity according to LC/MS neutral 90.2%).

1H-NMR (D6-DMSO) δ ppm: 7.81-7.79 (m, 1H), 7.49-7.47 (m, 1H), 4.04-3.90 (m, 4H), 3.04 (s, 3H), 2.46 (s, 3H), 1.17 (t, 3H)

logP (HCOOH): 3.32 logP (neutral): 3.42

### Preparation of 6-chloro-3-ethyl-5-fluoro-1-methylpyrimidine-2,4(1H,3H)-dione (II-01)

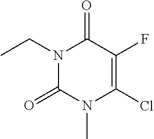

120 mg (0.623 mmol) of 6-chloro-3-ethyl-5-fluoropyrimidine-2,4(1H,3H)-dione (Aurora Fine Chemicals, San Diego, USA; CAS-Reg. No. 13593-35-6) were dissolved in 10 ml of dimethylformamide and, after addition of 250 mg (1.81 mmol) of potassium carbonate and 250 mg of caesium carbonate (0.767 mmol), 250 mg (1.76 mmol) of iodomethane were added and the mixture was stirred at room temperature for 18 h. The solvent was then distilled off under reduced pressure and the residue was treated with water and dichloromethane. The combined organic phases were, after filtration through a layer of silica gel, dried over MgSO₄ and freed from the solvent under reduced pressure. 96 mg of product (75% of theory, purity according to LC/MC 86%) remained.

1H-NMR (D6-DMSO) δ ppm: 3.88-3.82 (q, 2H), 3.43 (s, 3H), 1.11 (t, 3H)

logP (HCOOH): 1.31

### Preparation Example 4: 6-{2,4-Dimethyl-5-[(2,2,2-trifluoroethyl)sulphanyl]phenyl}-2,4-dimethyl-1,2,4-triazine-3,5(2H,4H)-dione (1-4)

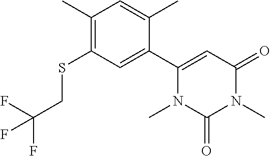 [SMILES: Cc1cc(C)c(-c2cc(=O)n(C)c(=O)n2C)cc1SCC(F)(F)F]

80 mg (0.458 mmol) of 6-chloro-1,3-dimethylpyrimidine-2,4(1H,3H)-dione and 50 mg (0.043 mmol) of tetrakis(triphenylphosphine)palladium in 20 ml of dioxane were heated under reflux with 160 mg (0.462 mmol) of 2-{2,4-dimethyl-5-[(2,2,2-trifluoroethyl)sulphanyl]phenyl}-4,4,5,5-tetramethyl-1,3,2-dioxaborolane and 3 ml of 2-molar sodium carbonate solution for 18 h. After cooling, water was added and the reaction mixture was extracted with dichloromethane. The combined organic phases were, after filtration through a layer of silica gel, dried over MgSO₄ and freed from the solvent under reduced pressure. The residue was purified by column chromatography on a 10 g SNAP column (Biotage) using ethyl acetate/cyclohexane (gradient 10-80% ethyl acetate) as mobile phase. This gave 7 mg of product as a white solid (4.3% of theory, purity according to NMR 85%).

1H-NMR (D₆-DMSO) δ ppm: 7.47 (s, 1H), 7.27 (s, 1H), 5.56 (s, 1H), 4.03-3.94 (m, 2H), 3.23 (s, 3H), 2.93 (s, 3H), 2.37 (s, 3H), 2.16 (s, 3H)

logP (HCOOH): 2.98

### Preparation Example 5: 6-{2-Fluoro-4-methyl-5-[(2,2,2-trifluoroethyl)sulphanyl]phenyl}-3-methylpyrimidine-2,4(1H,3H)-dione (1-13)

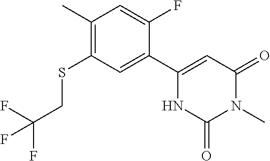 [SMILES: Cc1cc(F)c(-c2cc(=O)n(C)c(=O)[nH]2)cc1SCC(F)(F)F]

A mixture of 600 mg (1.773 mmol) of ethyl 3-{2-fluoro-4-methyl-5-[(2,2,2-trifluoroethyl)sulphanyl]phenyl}-3-oxopropanoate (VI-01) and 175 mg (2.362 mmol) of N-methylurea was stirred at 140° C. for 12 h. The crude product was purified directly by column chromatography on a 50 g SNAP column (Biotage) using ethyl acetate/cyclohexane (gradient 5-80% ethyl acetate) as mobile phase. This gave 103 mg of product as a white solid (16.7% of theory, purity according to LC/MS 99.3%).

1H-NMR (D₆-DMSO) δ ppm: 11.46 (s, 1H), 7.74-7.73 (m, 1H), 7.37-7.35 (m, 1H), 5.86 (s, 1H), 4.09-4.04 (q, 2H), 3.17 (s, 3H), 2.43 (s, 3H)

13C-NMR (D₆-DMSO) δ ppm: 162.7, 158.0, 151.5, 145.0, 144.3, 128.9, 126.0, 118.4, 118.1, 100.4, 35.1, 26.4, 20.0

logP (HCOOH): 2.38 logP (neutral): 2.25

### Preparation of ethyl 3-{2-fluoro-4-methyl-5-[(2,2,2-trifluoroethyl)sulphanyl]phenyl}-3-oxopropanoate (VI-01)

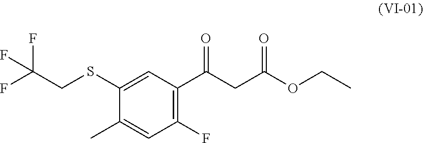 [SMILES: CCOC(=O)CC(=O)c1cc(SCC(F)(F)F)c(C)cc1F]

33.9 g (0.20 mol) of potassium monoethyl malonate were initially charged in a 2 l flask under argon, and 300 ml of acetonitrile were added. The mixture was cooled to 0° C., after which 22.2 g (0.22 mol) of triethylamine and 23.7 g (0.25 mol) of magnesium chloride were added. The reaction mixture was stirred at 20-25° C. for 3 h. The resulting suspension was cooled to 0° C., and 28.6 g (0.10 mol) of 2-fluoro-4-methyl-5-(2,2,2-trifluoroethyl)sulphanylbenzoyl chloride were added dropwise a little at a time. After subsequent addition of 2.02 g (0.02 mol) of triethylamine, the mixture was stirred at room temperature overnight. The mixture was added to 500 ml of a 10% strength HCl solution and stirred for another 2 h. The solid formed was filtered off with suction and air-dried. This gave 31.9 g of product (94% of theory, purity >95% according to 1H-NMR).

1H-NMR (CDCl₃) δ ppm: 12.70 (s, 1H), 8.05-8.03 (m, 1H), 7.03-7.00 (m, 1H), 5.81 (s, 1H), 4.30-4.24 (q, 2H), 3.41-3.34 (q, 2H), 2.50 (s, 3H), 1.34 (t, 3H)

logP (HCOOH): 3.57 logP (neutral): 3.59

### Preparation Example 6: 6-{2-Fluoro-4-methyl-5-[(2,2,2-trifluoroethyl)sulphanyl]phenyl}-2-thioxo-2,3-dihydropyrimidin-4(1H)-one (1-14)

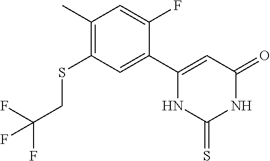 [SMILES: Cc1cc(F)c(-c2cc(=O)[nH]c(=S)[nH]2)cc1SCC(F)(F)F]

A mixture of 600 mg (1.773 mmol) of ethyl 3-{2-fluoro-4-methyl-5-[(2,2,2-trifluoroethyl)sulphanyl]phenyl}-3-oxopropanoate (VI-01) and 175 mg (2.299 mmol) of thiourea was stirred at 140° C. for 12 h. The crude product was purified directly by column chromatography on a 50 g SNAP column (Biotage) using ethyl acetate/cyclohexane (gradient 5-50% ethyl acetate) as mobile phase. This gave 48 mg of product as a beige solid (7.7% of theory, purity according to LC/MS 92.3%), in addition, 115 mg (18.5% of theory, purity according to LC/MS 78.9%) of a further fraction were isolated. 1H-NMR (D₆-DMSO) δ ppm: 12.61 (s, 1H), 12.58 (s, 1H), 7.74-7.72 (m, 1H), 7.36-7.33 (m, 1H), 6.03 (s, 1H), 4.09-4.01 (q, 2H), 2.43 (s, 3H)

logP (HCOOH): 2.34 logP (neutral): 1.54

### Preparation Example 7: 5-Chloro-6-{2-fluoro-4-methyl-5-[(2,2,2-trifluoroethyl)sulphanyl]phenyl}-1,3-dimethylpyrimidine-2,4(1H,3H)-dione (1-16)

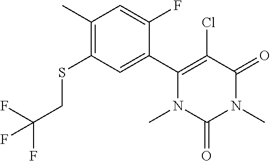 [SMILES: Cc1cc(F)c(-c2c(Cl)c(=O)n(C)c(=O)n2C)cc1SCC(F)(F)F]

65 mg (0.482 mmol) of sulphuryl chloride, dissolved in 3 ml of acetonitrile, were slowly added dropwise to a solution of 109 mg (0.301 mmol) of 6-{2-fluoro-4-methyl-5-[(2,2,2-trifluoroethyl)sulphanyl]phenyl}-1,3-dimethylpyrimidine-2,4(1H,3H)-dione (1-2) in 5 ml of acetonitrile and 50 mg of acetic acid, and the mixture was then stirred at 80° C. for 12 h. After cooling, water was added and the reaction mixture was extracted with dichloromethane. The combined organic phases were, after filtration through a layer of silica gel, dried over MgSO₄ and freed from the solvent under reduced pressure. The residue was purified by column chromatography on a 10 g SNAP column (Biotage) using ethyl acetate/cyclohexane (gradient 10-80% ethyl acetate) as mobile phase. This gave 8 mg of product as a white solid (6.7% of theory, purity according to LC/MS 100%).

1H-NMR (D₆-DMSO) δ ppm: 7.74-7.73 (m, 1H), 7.48-7.46 (m, 1H), 4.02-3.94 (m, 2H), 3.30 (s, 3H), 3.05 (s, 3H), 2.46 (s, 3H)

logP (HCOOH): 3.15

The following compounds of the formula (I) were obtained analogously to the examples and in accordance with the preparation processes described above:

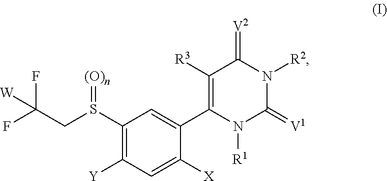

where V²=O and W=F

| Ex. | R¹ | R² | R³ | X | Y | n | V¹ |
|---|---|---|---|---|---|---|---|
| I-5 | n-C₃H₇ | n-C₃H₇ | H | F | CH₃ | 0 | O |
| I-6 | i-Pr | i-Pr | H | F | CH₃ | 0 | O |
| I-7 | C₂H₅ | C₂H₅ | H | F | CH₃ | 0 | O |
| I-8 | H | C₂H₅ | F | F | CH₃ | 0 | O |
| I-9 | i-Pr | i-Pr | H | F | CH₃ | 1 | O |
| I-10 | C₂H₅ | C₂H₅ | H | F | CH₃ | 1 | O |
| I-11 | CH₃ | C₂H₅ | F | F | CH₃ | 1 | O |
| I-12 | CH₃ | CH₃ | H | CH₃ | CH₃ | 1 | O |
| I-15 | CH₃ | F₃C—CH₂ | H | F | CH₃ | 0 | S |
| I-17 | H | CH₃ | H | F | CH₃ | 1 | O |

NMR data and logP values: logP (HCOOH) = logP[a] logP (neutral) = logP[b]

| Ex. No. | logP [b] | logP [a] | NMR [δ ppm] |
|---|---|---|---|
| I-5 | 4.16 | 4.26 | 1H-NMR (D₆-DMSO): 7.79-7.77 (m, 1H), 7.42-7.39 (m, 1H), 5.71 |
| | | | (s, 1H), 4.06-3.63 (m, 6H), 2.44 (s, 3H) 1.58 (m, 2H), 1.37 (m, 2H), 0.89 |
| | | | (t, 3H), 0.61 (t, 3H) |
| I-6 | 4.42 | 4.52 | 1H-NMR (D₆-DMSO): 7.75-7.73 (m, 1H), 7.41-7.38 (m, 1H), 5.56 |
| | | | (s, 1H), 5.10-5.05 (m, 1H), 4.13-3.98 (m, 2H), 3.77-3.72 (m, 1H), 2.42 |
| | | | (s, 3H), 1.41-1.30 (4xd, 12H) |
| I-7 | 3.47 | 3.54 | 1H-NMR (D₆-DMSO): 7.77-7.75 (m, 1H), 7.42-7.40 (m, 1H), 5.70 |
| | | | (s, 1H), 4.11-3.87 (m, 4H), 3.72-3.65 (m, 1H), 3.48-3.41 (m, 1H), 2.43 |
| | | | (s, 3H), 1.14 (t, 3H), 0.96 (t, 3H) |
| I-8 | | 2.72 | 1H-NMR (D₆-DMSO): 7.78-7.77 (m, 1H), 7.40-7.37 (m, 1H), 4.05- |
| | | | 3.98 (q, 2H), 3.86 (m, 2H), 2.45 (s, 3H), 1.15 (t, 3H). |
| I-9 | 3.10 | 3.19 | 1H-NMR (D₆-DMSO): 7.94-7.92 (m, 1H), 7.53-7.50 (m, 1H), 5.71 |
| | | | (s, 1H), 5.11-5.04 (m, 1H), 4.27-4.11 (m, 2H), 3.77-3.73 (m, 1H), 2.45 |
| | | | (s, 3H), 1.42-1.33 (4xd, 12H) |
| I-10 | 2.28 | 2.37 | 1H-NMR (D₆-DMSO): 7.97-7.95 (m, 1H), 7.55-7.52 (m, 1H), 5.76 |
| | | | (s, 1H), 4.22-4.14 (m, 2H), 3.92-3.87 (m, 2H), 3.65 (m, 2H), 2.46 |
| | | | (s, 3H), 1.15 (t, 3H), 0.98 (broad, 3H) |
| I-11 | 2.19 | 2.20 | 1H-NMR (D₆-DMSO): 8.07-8.05 (m, 1H), 7.61-7.58 (m, 1H), 4.35- |
| | | | 3.99 (m, 2H), 3.96-3.90 (q, 2H), 3.08 (s, 3H), 2.47 (s, 3H), 1.17 (t, 3H) |
| I-12 | 1.82 | 1.89 | 1H-NMR (D₆-DMSO): 7.74 (s, 1H), 7.38 (s, 1H), 5.60 (s, 1H), 4.17- |
| | | | 4.06 (m, 2H), 3.24 (s, 3H), 2.94 (s, 3H), 2.40 (s, 3H), 2.26 (s, 3H) |
| I-15 | | 4.34 | 1H-NMR (D₆-DMSO): 7.78-7.77 (m, 1H), 7.45-7.43 (m, 1H), 6.27 |
| | | | (s, 1H), 5.45-5.31 (m, 2H), 4.08-4.02 (m, 2H), 3.49 (s, 3H), 2.44 (s, 3H) |
| | | | 13C-NMR (D₆-DMSO) δ ppm: 178.9, 158.4, 157.1, 149.9, 144.3, |
| | | | 132.3, 129.7, 126.0, 124.3, 119.7, 117.9, 107.5, 47.6, 42.5, 34.8, 20.3 |
| I-17 | 1.43 | 1.41 | 1H-NMR (D₆-DMSO): 11.54 (s, 1H), 7.94-7.92 (m, 1H), 7.53-7.50 |
| | | | (m, 1H), 5.76 (broad, 1H), 4.22-4.09 (m, 2H), 3.02 (s, 3H), 2.45 (s, 3H) |

### Further Examples of the Preparation of Starting Materials

### Preparation of Compounds of the Formula (II):

### Preparation of 6-chloro-3-ethyl-1,5-dimethylpyrimidine-2,4(1H,3H)-dione (11-02)

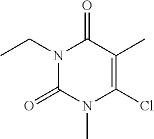

120 mg (0.636 mmol) of 6-chloro-3-ethyl-5-methylpyrimidine-2,4(1H,3H)-dione (CAS-Reg. No. 1565665-59-9) were dissolved in 5 ml of dimethylformamide and, after addition of 250 mg (1.81 mmol) of potassium carbonate and 250 mg of caesium carbonate (0.767 mmol), 260 mg (1.83 mmol) of iodomethane were added and the mixture was stirred at room temperature for 18 h. The solvent was then distilled off under reduced pressure and the residue was treated with water and dichloromethane. The combined organic phases were, after filtration through a layer of silica gel, dried over MgSO₄ and freed from the solvent under reduced pressure. 95 mg of product (73.7% of theory, purity according to LC/MS 73%) remained.

1H-NMR (D6-DMSO) δ ppm: 3.86-3.83 (q, 2H), 3.46 (s, 3H), 1.96 (s, 3H), 1.09 (t, 3H)

logP (HCOOH): 1.59

### Use Examples

1. Boophilus microplus—Injection Test
Solvent: dimethyl sulphoxide

To produce a suitable preparation of active compound, 10 mg of active compound are mixed with 0.5 ml of solvent and the concentrate is diluted with solvent to the desired concentration.

1 μl of the active compound solution is injected into the abdomen of 5 engorged adult female cattle ticks (Boophilus microplus). The animals are transferred into dishes and kept in a climate-controlled room.

Efficacy is assessed after 7 days by laying of fertile eggs. Eggs which are not visibly fertile are stored in a climate-controlled cabinet until the larvae hatch after about 42 days. An efficacy of 100% means that none of the ticks has laid any fertile eggs; 0% means that all the eggs are fertile.

In this test, for example, the following compounds from the preparation examples showed an efficacy of 100% at an application rate of 20 Gg/animal: I-1, I-2, I-9, I-10, I-11, I-12

2. Meloidogyne incognita Test
Solvent: 125.0 parts by weight of acetone

To produce a suitable preparation of active compound, 1 part by weight of active compound is mixed with the stated amount of solvent and the concentrate is diluted with water to the desired concentration.

Vessels are filled with sand, active compound solution, an egg/larvae suspension of the southern root-knot nematode (Meloidogyne incognita) and lettuce seeds. The lettuce seeds germinate and the plants develop. The galls develop on the roots.

After 14 days, the nematicidal efficacy in % is determined by the formation of galls. 100% means that no galls were found; 0% means that the number of galls on the treated plants corresponds to the untreated control.

In this test, for example, the following compounds from the preparation examples showed an efficacy of 100% at an application rate of 20 ppm: I-1

In this test, for example, the following compounds from the preparation examples showed an efficacy of 90% at an application rate of 20 ppm: I-2

3. Phaedon cochleariae—Spray Test
Solvent: 78.0 parts by weight of acetone
1.5 parts by weight of dimethylformamide
   Emulsifier: alkylaryl polyglycol ether

To produce a suitable preparation of active compound, 1 part by weight of active compound is dissolved using the stated parts by weight of solvent and made up with water containing an emulsifier concentration of 1000 ppm until the desired concentration is attained. To produce further test concentrations, the preparation is diluted with emulsifier-containing water.

Discs of Chinese cabbage leaves (Brassica pekinensis) are sprayed with an active compound preparation of the desired concentration and, after drying, populated with larvae of the mustard beetle (Phaedon cochleariae).

After 7 days, the efficacy in % is determined. 100% means that all the beetle larvae have been killed; 0% means that no beetle larvae have been killed.

In this test, for example, the following compounds from the preparation examples showed an efficacy of 100% at an application rate of 500 g/ha: I-15

In this test, for example, the following compounds from the preparation examples showed an efficacy of 83% at an application rate of 500 g/ha: I-2

4. Tetranychus urticae—Spray Test, OP-Resistant
Solvent: 78.0 parts by weight of acetone
1.5 parts by weight of dimethylformamide
   Emulsifier: alkylaryl polyglycol ether

To produce a suitable preparation of active compound, 1 part by weight of active compound is dissolved using the stated parts by weight of solvent and made up with water containing an emulsifier concentration of 1000 ppm until the desired concentration is attained. To produce further test concentrations, the preparation is diluted with emulsifier-containing water.

Discs of bean leaves (Phaseolus vulgaris) infested with all stages of the greenhouse red spider mite (Tetranychus urticae) are sprayed with an active compound preparation of the desired concentration.

After 6 days, the efficacy in % is determined. 100% means that all the spider mites have been killed; 0% means that none of the spider mites have been killed.

In this test, for example, the following compounds from the preparation examples showed an efficacy of 100% at an application rate of 500 g/ha: I-1, I-2, I-6, I-9, I-10, I-11, I-12

In this test, for example, the following compounds from the preparation examples showed an efficacy of 90% at an application rate of 500 g/ha: I-5, I-7, I-15, I-16

5. Tetranychus urticae-Spray Test, OP-Resistant
Solvent: 7 parts by weight of dimethylformamide
Emulsifier: alkylaryl polyglycol ether

To produce a suitable preparation of active compound, 1 part by weight of active compound is dissolved using the stated parts by weight of solvent and made up with water containing an emulsifier concentration of 1000 ppm until the desired concentration is attained. To produce further test concentrations, the preparation is diluted with emulsifier-containing water. If the addition of ammonium salts or/and penetrants is required, these are each added in a concentration of 1000 ppm to the preparation solution.

Bean plants (Phaseolus vulgaris) heavily infested by all stages of the greenhouse red spider mite (Tetranychus urticae) are sprayed with an active compound preparation of the desired concentration.

After 7 days, the efficacy in % is determined. 100% means that all the spider mites have been killed; 0% means that none of the spider mites have been killed.

In this test, for example, the following compounds from the preparation examples showed an efficacy of 100% at an application rate of 20 ppm: I-1, I-2, I-7, I-10, I-15

6. Tetranychus urticae—Drench Test, OP-Resistant
Solvent: 7 parts by weight of dimethylformamide
Emulsifier: 2 parts by weight of alkylaryl polyglycol ether

To produce a suitable preparation of active compound, 1 part by weight of active compound is mixed with the stated amounts of solvent and emulsifier, and the concentrate is diluted with water to the desired concentration, where the volume of soil to be drenched has to be taken into account. Here, it has to be made sure that a concentration of 40 ppm of emulsifier in the soil is not exceeded. To produce further test concentrations, the mixture is diluted with water.

Bean plants (Phaseolus vulgaris) in pots filled with soil, which plants are heavily infested by all stages of the greenhouse red spider mite (Tetranychus urticae), are watered with an active compound preparation of the desired concentration.

After 14 days, the efficacy in % is assessed. 100% means that all spider mites have been killed; 0% means that none of the spider mites have been killed.

In this test, for example, the following compounds from the preparation examples showed an efficacy of 100% at an application rate of 4 ppm: I-1, I-15

In this test, for example, the following compounds from the preparation examples showed an efficacy of 99% at an application rate of 4 ppm: I-2

In this test, for example, the following compounds from the preparation examples showed an efficacy of 95% at an application rate of 4 ppm: 1-10

7. Meloidogyne incognita—Test
Solvent: 7 parts by weight of dimethylformamide
Emulsifier: 2.5 parts by weight of alkylaryl polyglycol ether

To produce a suitable preparation of active compound, 1 part by weight of active compound is mixed with the stated amounts of solvent and emulsifier, and the concentrate is diluted with water to the desired concentration, where the volume of soil to be drenched has to be taken into account. Here, it has to be made sure that a concentration of 20 ppm of emulsifier in the soil is not exceeded. To produce further test concentrations, the mixture is diluted with water.

Pots filled with soil (loamy sand) are watered with the active compound solution. An egg/larvae suspension of the southern root-knot nematode (Meloidogyne incognita) is added, lettuce seeds are scattered on the surface of the soil and covered with quartz sand. The lettuce seeds germinate and the plants develop. The galls develop on the roots.

After 21 days, the nematicidal efficacy in % is determined by the formation of galls. 100% means that no galls were found; 0% means that the number of galls on the treated plants corresponds to that of the untreated control.

In this test, for example, the following compounds of the preparation examples showed an efficacy of 90% at an application rate of 8 ppm: I-1

8. Panonychus ulmi—Spray Test
Solvent: 7 parts by weight of dimethylformamide
Emulsifier: alkylaryl polyglycol ether

To produce a suitable preparation of active compound, 1 part by weight of active compound is dissolved in the parts by weight of solvent stated and filled with water containing an emulsifier concentration of 1000 ppm until the desired concentration is reached. To prepare further test concentrations, the mixture is diluted with emulsifier-containing water. If the addition of ammonium salts and/or penetrants (rapeseed oil methyl ester) is required, these are, after dilution of the finished preparation solution, added using a pipette, at a concentration of in each case 1000 ppm.

Plum trees (Prunus domestica), infested by a mixed population of the European red mite (Panonychus ulmi) are treated by spraying with the active compound preparation of the desired concentration.

After 14 days, the kill in % is determined. 100% means that all spider mites have been killed; 0% means that none of the spider mites have been killed.

In this test, for example, the following compounds of the preparation examples showed an efficacy of 100% at an application rate of 20 ppm: I-1

1. Compound of the formula (I) 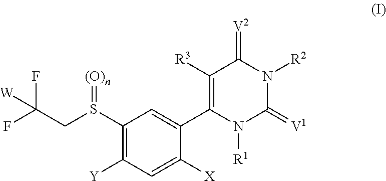
in which
V¹ and V² independently of one another represent oxygen or represent sulphur;
R³ represents hydrogen, alkyl, halogen, haloalkyl, amino, cyano, carbamoyl, nitro, hydroxy, hydroalkyl, alkylthiosulphanyl, alkylsulphanyl, alkylsulphinyl, alkylsulphonyl, haloalkylsulphanyl, haloalkylsulphinyl, haloalkylsulphonyl, cycloalkyl, alkylcarbonyl, haloalkylcarbonyl, alkoxycarbonyl, alkoxyalkyl, haloalkoxy or alkoxy;
R¹ and R² independently of one another represent hydrogen or alkyl; or
represent alkenyl, alkynyl, cycloalkyl, cycloalkylalkyl, cycloalkenyl, cyanoalkyl, hydroxyalkyl, alkylcarbonyl, alkoxycarbonyl, alkoxycarbonylalkyl, haloalkylcarbonyl, alkoxyalkyl, haloalkoxyalkyl, alkoxy, haloalkoxy, haloalkyl, haloalkenyl or haloalkynyl, where the radicals mentioned above may optionally be substituted by fluorine, chlorine, bromine, iodine, alkyl, cycloalkyl, cyano, nitro, alkoxy, haloalkyl or haloalkoxy;
W represents hydrogen or halogen;
n represents the number 0, 1 or 2;
Y represents hydrogen, halogen, cyano, (C₁-C₆)-alkyl, (C₃-C₆)-cycloalkyl, (C₁-C₆)-haloalkyl, (C₁-C₆)-alkoxy or (C₁-C₆)-haloalkoxy;
X represents hydrogen, halogen, cyano, (C₁-C₆)-alkyl, (C₁-C₆)-haloalkyl or (C₁-C₆)-alkoxy.

2. Compound according to claim 1, in which
V¹ and V² independently of one another represent oxygen or represent sulphur;
R³ represents hydrogen, halogen, amino, cyano, carbamoyl, nitro, hydroxy, (C₁-C₆)-alkyl, (C₁-C₆)hydroxyalkyl, (C₁-C₆)-haloalkyl, (C₁-C₆)alkylthiosulphanyl, (C₁-C₆)-alkylsulphanyl, (C₁-C₆)-alkylsulphinyl, (C₁-C₆)-alkylsulphonyl, (C₁-C₆)-haloalkylsulphanyl, (C₁-C₆)-haloalkylsulphinyl, (C₁-C₆)-haloalkylsulphonyl, (C₃-C₆)-cycloalkyl, (C₁-C₆)-alkylcarbonyl, (C₁-C₆)-haloalkylcarbonyl, (C₁-C₆)-alkoxycarbonyl, (C₁-C₆)alkoxyalkyl, (C₁-C₆)haloalkoxy or (C₁-C₆)-alkoxy;
R¹ and R² independently of one another represent hydrogen or alkyl; or represent alkenyl, alkynyl, cycloalkyl, cycloalkylalkyl, cycloalkenyl, cyanoalkyl, hydroxyalkyl, alkylcarbonyl, haloalkylcarbonyl, alkoxyalkyl, haloalkoxyalkyl, alkoxy or haloalkoxy, where the radicals mentioned above may optionally be substituted by fluorine, chlorine, bromine, iodine, (C₁-C₆)-alkyl, (C₃-C₆)-cycloalkyl, cyano, nitro, (C₁-C₆)-alkoxy, (C₁-C₆)-haloalkyl or (C₁-C₆)-haloalkoxy; or
represent haloalkyl, haloalkenyl or haloalkynyl;
W represents hydrogen or halogen;
n represents the number 0 or 1;
Y represents hydrogen, halogen, (C₁-C₆)-alkyl, (C₃-C₆)-cycloalkyl, (C₁-C₆)-haloalkyl, (C₁-C₆)-alkoxy or (C₁-C₆)-haloalkoxy;
X represents hydrogen, halogen, (C₁-C₆)-alkyl, (C₁-C₆)-haloalkyl or (C₁-C₆)-alkoxy.

3. Compound according to claim 1, in which
V¹ and V² independently of one another represent oxygen or represent sulphur;
R³ represents hydrogen, amino, halogen, cyano, (C₁-C₄)-alkyl, (C₁-C₄)-haloalkyl, (C₃-C₆)-cycloalkyl, (C₁-C₄)-alkylcarbonyl, (C₁-C₄)-haloalkylcarbonyl, (C₁-C₄)-alkoxycarbonyl or (C₁-C₄)-alkoxy;
R¹ and R² independently of one another represent hydrogen, (C₁-C₄)-alkyl, cyano-(C₁-C₄)-alkyl, (C₁-C₄)-haloalkyl, (C₁-C₄)-alkoxy-(C₁-C₄)-alkyl, (C₁-C₄)-haloalkoxy-(C₁-C₄)-alkyl, (C₁-C₄)-alkoxy or (C₁-C₄)-haloalkoxy; or
represent (C₃-C₆)-cycloalkyl or (C₃-C₆)-cycloalkyl-(C₁-C₂)-alkyl, where the radicals mentioned above may optionally be substituted by fluorine, chlorine, bromine, iodine, (C₁-C₃)-alkyl, (C₃-C₆)-cycloalkyl, cyano, nitro, (C₁-C₄)-alkoxy, (C₁-C₃)-haloalkyl or (C₁-C₃)-haloalkoxy;
W represents hydrogen, chlorine or fluorine;
n represents the number 0 or 1;
Y represents halogen, (C₁-C₄)-alkyl, (C₃-C₆)-cycloalkyl, (C₁-C₄)-haloalkyl, (C₁-C₄)-alkoxy or (C₁-C₄)-haloalkoxy;
X represents hydrogen, halogen, (C₁-C₄)-alkyl, (C₁-C₄)-haloalkyl or (C₁-C₄)-alkoxy.

4. Compound according to claim 1, in which
V¹ and V² independently of one another represent oxygen or represent sulphur;
R³ represents hydrogen, chlorine, fluorine, methyl, ethyl, methoxy, cyclopropyl, cyano or trifluoromethyl;
R¹ and R² independently of one another represent hydrogen, methyl, ethyl, n-propyl, isopropyl, n-butyl, sec-butyl, tert-butyl, CH₂CH(CH₃)₂, CH₂CN, CH₂CF₃, CH₂CHF₂, CH₂CH₂OCH₃, cyclopropyl, cyclobutyl or cyclopropylmethyl;
W represents fluorine;
n represents the number 0 or 1;
Y represents fluorine, chlorine, bromine, methyl, trifluoromethyl or methoxy;
X represents hydrogen, chlorine, fluorine or methyl.

5. Compound according to claim 1, in which
V¹ and V² independently of one another represent oxygen or represent sulphur;
R³ represents hydrogen, fluorine or chlorine;
R¹ and R² independently of one another represent hydrogen, methyl, ethyl, n-propyl, isopropyl or CH₂CF₃;
W represents fluorine;
n represents the number 0 or 1;
Y represents methyl;
X represents fluorine or methyl.

6. Compound according to claim 1 according to formula (I-A) 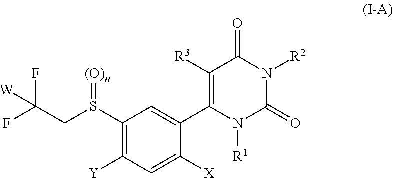
in which
V¹ and V² independently of one another represent oxygen or represent sulphur;
R³ represents hydrogen, alkyl, halogen, haloalkyl, amino, cyano, carbamoyl, nitro, hydroxy, hydroalkyl, alkylthiosulphanyl, alkylsulphanyl, alkylsulphinyl, alkylsulphonyl, haloalkylsulphanyl, haloalkylsulphinyl, haloalkylsulphonyl, cycloalkyl, alkylcarbonyl, haloalkylcarbonyl, alkoxycarbonyl, alkoxyalkyl, haloalkoxy or alkoxy;
R¹ and R² independently of one another represent hydrogen or alkyl; or
represent alkenyl, alkynyl, cycloalkyl, cycloalkylalkyl, cycloalkenyl, cyanoalkyl, hydroxyalkyl, alkylcarbonyl, alkoxycarbonyl, alkoxycarbonylalkyl, haloalkylcarbonyl, alkoxyalkyl, haloalkoxyalkyl, alkoxy, haloalkoxy, haloalkyl, haloalkenyl or haloalkynyl, where the radicals mentioned above may optionally be substituted by fluorine, chlorine, bromine, iodine, alkyl, cycloalkyl, cyano, nitro, alkoxy, haloalkyl or haloalkoxy;
W represents hydrogen or halogen;
n represents the number 0, 1 or 2;
Y represents hydrogen, halogen, cyano, (C₁-C₆)-alkyl, (C₃-C₆)-cycloalkyl, (C₁-C₆)-haloalkyl, (C₁-C₆)-alkoxy or (C₁-C₆)-haloalkoxy;
X represents hydrogen, halogen, cyano, (C₁-C₆)-alkyl, (C₁-C₆)-haloalkyl or (C₁-C₆)-alkoxy.

7. Compound according to claim 1 according to formula (I-B) 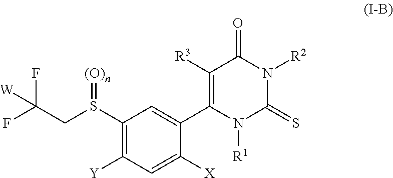
in which
V¹ and V² independently of one another represent oxygen or represent sulphur;
R³ represents hydrogen, alkyl, halogen, haloalkyl, amino, cyano, carbamoyl, nitro, hydroxy, hydroalkyl, alkylthiosulphanyl, alkylsulphanyl, alkylsulphinyl, alkylsulphonyl, haloalkylsulphanyl, haloalkylsulphinyl, haloalkylsulphonyl, cycloalkyl, alkylcarbonyl, haloalkylcarbonyl, alkoxycarbonyl, alkoxyalkyl, haloalkoxy or alkoxy;
R¹ and R² independently of one another represent hydrogen or alkyl: or
represent alkenyl, alkynyl, cycloalkyl, cycloalkylalkyl, cycloalkenyl, cyanoalkyl, hydroxyalkyl, alkylcarbonyl, alkoxycarbonyl, alkoxycarbonylalkyl, haloalkylcarbonyl, alkoxyalkyl, haloalkoxyalkyl, alkoxy, haloalkoxy, haloalkyl, haloalkenyl or haloalkynyl, where the radicals mentioned above may optionally be substituted by fluorine, chlorine, bromine, iodine, alkyl, cycloalkyl, cyano, nitro, alkoxy, haloalkyl or haloalkoxy;
W represents hydrogen or halogen;
n represents the number 0, 1 or 2;
Y represents hydrogen, halogen, cyano, (C₁-C₆)-alkyl, (C₃-C₆)-cycloalkyl, (C₁-C₆)-haloalkyl, (C₁-C₆)-alkoxy or (C₁-C₆)-haloalkoxy;
X represents hydrogen, halogen, cyano, (C₁-C₆)-alkyl, (C₁-C₆)-haloalkyl or (C₁-C₆)-alkoxy.

8. Compound according to claim 1 according to formula (I-C) 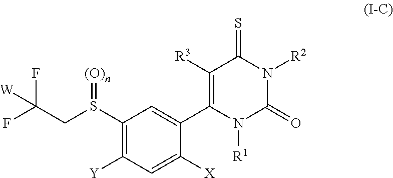
in which
V¹ and V² independently of one another represent oxygen or represent sulphur;
R³ represents hydrogen, alkyl, halogen, haloalkyl, amino, cyano, carbamoyl, nitro, hydroxy, hydroalkyl, alkylthiosulphanyl, alkylsulphanyl, alkylsulphinyl, alkylsulphonyl, haloalkylsulphanyl, haloalkylsulphinyl, haloalkylsulphonyl, cycloalkyl, alkylcarbonyl, haloalkylcarbonyl, alkoxycarbonyl, alkoxyalkyl, haloalkoxy or alkoxy;
R¹ and R² independently of one another represent hydrogen or alkyl: or
represent alkenyl, alkynyl, cycloalkyl, cycloalkylalkyl, cycloalkenyl, cyanoalkyl, hydroxyalkyl, alkylcarbonyl, alkoxycarbonyl, alkoxycarbonylalkyl, haloalkylcarbonyl, alkoxyalkyl, haloalkoxyalkyl, alkoxy, haloalkoxy, haloalkyl, haloalkenyl or haloalkynyl, where the radicals mentioned above may optionally be substituted by fluorine, chlorine, bromine, iodine, alkyl, cycloalkyl, cyano, nitro, alkoxy, haloalkyl or haloalkoxy;
W represents hydrogen or halogen;
n represents the number 0, 1 or 2;
Y represents hydrogen, halogen, cyano, (C₁-C₆)-alkyl, (C₃-C₆)-cycloalkyl, (C₁-C₆)-haloalkyl, (C₁-C₆)-alkoxy or (C₁-C₆)-haloalkoxy;
X represents hydrogen, halogen, cyano, (C₁-C₆)-alkyl, (C₁-C₆)-haloalkyl or (C₁-C₆)-alkoxy.

9. Compound according to claim 1 according to formula (I-D) 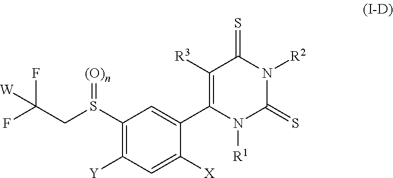
in which
V¹ and V² independently of one another represent oxygen or represent sulphur;
R³ represents hydrogen, alkyl, halogen, haloalkyl, amino, cyano, carbamoyl, nitro, hydroxy, hydroalkyl, alkylthiosulphanyl, alkylsulphanyl, alkylsulphinyl, alkylsulphonyl, haloalkylsulphanyl, haloalkylsulphinyl, haloalkylsulphonyl, cycloalkyl, alkylcarbonyl, haloalkylcarbonyl, alkoxycarbonyl, alkoxyalkyl, haloalkoxy or alkoxy;
R¹ and R² independently of one another represent hydrogen or alkyl; or
represent alkenyl, alkynyl, cycloalkyl, cycloalkylalkyl, cycloalkenyl, cyanoalkyl, hydroxyalkyl, alkylcarbonyl, alkoxycarbonyl, alkoxycarbonylalkyl, haloalkylcarbonyl, alkoxyalkyl, haloalkoxyalkyl, alkoxy, haloalkoxy, haloalkyl, haloalkenyl or haloalkynyl, where the radicals mentioned above may optionally be substituted by fluorine, chlorine, bromine, iodine, alkyl, cycloalkyl, cyano, nitro, alkoxy, haloalkyl or haloalkoxy:
W represents hydrogen or halogen;
n represents the number 0, 1 or 2;
Y represents hydrogen, halogen, cyano, (C₁-C₆)-alkyl, (C₃-C₆)-cycloalkyl, (C₁-C₆)-haloalkyl, (C₁-C₆)-alkoxy or (C₁-C₆)-haloalkoxy;
X represents hydrogen, halogen, cyano, (C₁-C₆)-alkyl, (C₁-C₆)-haloalkyl or (C₁-C₆)-alkoxy.

10. Compound according to formula (II-01)

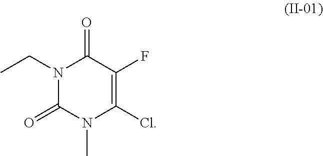

11. Compound according to formula (II-02)

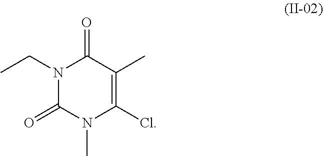

12. Compound according to formula (VI-01)

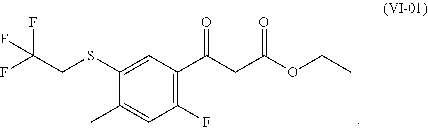 [SMILES: CCOC(=O)CC(=O)c1cc(SCC(F)(F)F)c(C)cc1F]

13. Formulation, optionally an agrochemical formulation, comprising at least one compound of the formula (I) according to claim 1.

14. Formulation according to claim 13, further comprising at least one extender and/or at least one surface-active substance.

15. Formulation according to claim 13, wherein the compound of the formula (I) is in a mixture with at least one further active compound.

16. Method for controlling pests, optionally one or more animal pests, comprising allowing a compound of the formula (I) according to claim 1 to act on the pests and/or a habitat thereof.

17. Method according to claim 16, wherein the pest is an animal pest and comprises an insect, an acarid or a nematode, or the pest is an insect, an acarid or a nematode.

18. A product comprising a compound of the formula (I) according to claim 1 for controlling one or more animal pests.

19. Product according to claim 18, wherein the animal pest comprises an insect, an acarid or a nematode, or the animal pest is an insect, an acarid or a nematode.

20. Product according to claim 18 adapted for use in crop protection.

21. Product according to claim 18 adapted for use in the field of animal health.

22. Method for protecting seed and/or a germinating plant from pests, optionally animal pests, comprising contacting seed with a compound of the formula (I) according to claim 1.

23. Seed obtained by a method according to claim 22.
